SEQUENCE LISTING (165 sequences)

SEQ ID NO: 1 - DNA, 3892 bp, Homo sapiens
  source 1..3892: mol_type = other DNA
gctcctctccccggaacaggcccccgacagctgctctcgggagccgcctcccgacacccgagccccgccggcgcctcccgctcccggctcccggctcctggctccctccgcctcccccgcccctcgccccgccgccgaagaggccccgctcccgggtcggacgcctgggtctgccgggaagagcgatgagaggtgtctgaaggtggctattcactgagcgatggggttggacttgaaggaatgccaagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaa

SEQ ID NO: 2 - protein, 913 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 3 - protein, 112 aa, synthetic construct
ELVMTQTPASVEAAVGGTVTIKCQASETISSRLAWYQQKPGQPPKLLIYQASKLPSGVPSRFKGTGSGTEYTLTISDLECADAATYYCQGCYYGGGSFYDSAFGGGTEVVVK

SEQ ID NO: 4 - protein, 116 aa, synthetic construct
QSVEESGGRLVTPGGSLTLTCTVSGFSLSSYDMSWVRQAPGKGLEWIGISWNSGFVDYASWAKGRFSISKTSTTVDLKITSPTTEDTATYFCARLGADDIYYFNLWGPGTLVTISS

SEQ ID NO: 5 - protein, 6 aa, synthetic construct
ETISSR

SEQ ID NO: 6 - skipped

SEQ ID NO: 7 - protein, 14 aa, synthetic construct
QGCYYGGGSFYDSA

SEQ ID NO: 8 - protein, 8 aa, synthetic construct
GFSLSSYD

SEQ ID NO: 9 - protein, 7 aa, synthetic construct
SWNSGFV

SEQ ID NO: 10 - protein, 13 aa, synthetic construct
ARLGADDIYYFNL

SEQ ID NO: 11 - protein, 107 aa, synthetic construct
DIQMTQSPSSVSASVGDRVTITCQASQSIGSVLAWYQQKPGKAPKLLISGVFDLASGVPSRFSGSGSGTDFTLTISSLQPEDFATYYCQYIPYGSSPFGGGTKVEIK

SEQ ID NO: 12 - protein, 107 aa, synthetic construct
DIQMTQSPSSVSASVGDRVTITCRASQSIGSVLAWYQQKPGKAPKLLIYGVFSLQSGVPSRFSGSGSGTDFTLTISSLQPEDFATYYCQYIPYGSSPFGGGTKVEIK

SEQ ID NO: 13 - protein, 121 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSS

SEQ ID NO: 14-16 - skipped (3 entries)

SEQ ID NO: 17 - protein, 6 aa, synthetic construct
QSIGSV

SEQ ID NO: 18 - skipped

SEQ ID NO: 19 - protein, 9 aa, synthetic construct
QYIPYGSSP

SEQ ID NO: 20 - protein, 8 aa, synthetic construct
GFSLNRYY

SEQ ID NO: 21 - protein, 7 aa, synthetic construct
ISYGDTT

SEQ ID NO: 22 - protein, 15 aa, synthetic construct
ARADTGDNGYLGLQL

SEQ ID NO: 23 - protein, 219 aa, synthetic construct
ELVMTQTPASVEAAVGGTVTIKCQASETISSRLAWYQQKPGQPPKLLIYQASKLPSGVPSRFKGTGSGTEYTLTISDLECADAATYYCQGCYYGGGSFYDSAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 446 aa, synthetic construct
QSVEESGGRLVTPGGSLTLTCTVSGFSLSSYDMSWVRQAPGKGLEWIGISWNSGFVDYASWAKGRFSISKTSTTVDLKITSPTTEDTATYFCARLGADDIYYFNLWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 6 aa, synthetic construct
QNIYSN

SEQ ID NO: 26 - protein, 6 aa, synthetic construct
QTISSW

SEQ ID NO: 27 - protein, 6 aa, synthetic construct
QTISSW

SEQ ID NO: 28 - protein, 7 aa, synthetic construct
QSVYSNY

SEQ ID NO: 29 - protein, 7 aa, synthetic construct
QSIGSTY

SEQ ID NO: 30 - protein, 6 aa, synthetic construct
QTIYSN

SEQ ID NO: 31 - protein, 8 aa, synthetic construct
KSVYNNNA

SEQ ID NO: 32 - protein, 6 aa, synthetic construct
QSISSY

SEQ ID NO: 33 - protein, 6 aa, synthetic construct
QTIYSS

SEQ ID NO: 34 - protein, 6 aa, synthetic construct
QSIGSY

SEQ ID NO: 35 - protein, 7 aa, synthetic construct
QIIDHDH

SEQ ID NO: 36 - protein, 8 aa, synthetic construct
QSVVDKNW

SEQ ID NO: 37 - protein, 8 aa, synthetic construct
KNIYNNNA

SEQ ID NO: 38 - protein, 8 aa, synthetic construct
QSVYSNNY

SEQ ID NO: 39 - protein, 8 aa, synthetic construct
ESVYSNNH

SEQ ID NO: 40 - protein, 7 aa, synthetic construct
QSIDNND

SEQ ID NO: 41 - protein, 6 aa, synthetic construct
QSISNH

SEQ ID NO: 42 - protein, 6 aa, synthetic construct
ESINSW

SEQ ID NO: 43 - protein, 8 aa, synthetic construct
ENLYKDNY

SEQ ID NO: 44 - protein, 12 aa, synthetic construct
QSGYYSSSTDIA

SEQ ID NO: 45 - protein, 12 aa, synthetic construct
QQGISSSNVDNV

SEQ ID NO: 46 - protein, 15 aa, synthetic construct
QCTYGSGSSSSYGCA

SEQ ID NO: 47 - protein, 11 aa, synthetic construct
QGGYSEIIENT

SEQ ID NO: 48 - protein, 12 aa, synthetic construct
LYGGFGSSTGDA

SEQ ID NO: 49 - protein, 10 aa, synthetic construct
QSYYGADDYT

SEQ ID NO: 50 - protein, 11 aa, synthetic construct
AGDYSDISDNN

SEQ ID NO: 51 - protein, 13 aa, synthetic construct
QNNNGFSGSNFNN

SEQ ID NO: 52 - protein, 13 aa, synthetic construct
QQGSSISNVDKNA

SEQ ID NO: 53 - protein, 13 aa, synthetic construct
QNNNGMTVSDFNA

SEQ ID NO: 54 - protein, 13 aa, synthetic construct
QNNNGMTVSDFNA

SEQ ID NO: 55 - protein, 10 aa, synthetic construct
AGDFESGVSG

SEQ ID NO: 56 - protein, 11 aa, synthetic construct
AADYSDISDNN

SEQ ID NO: 57 - protein, 9 aa, synthetic construct
LGGYNDDAN

SEQ ID NO: 58 - protein, 9 aa, synthetic construct
LGGYNDDAN

SEQ ID NO: 59 - protein, 11 aa, synthetic construct
QSYCVNTYGYT

SEQ ID NO: 60 - protein, 14 aa, synthetic construct
QSYYIINRSNYANS

SEQ ID NO: 61 - protein, 14 aa, synthetic construct
QSYYIINRSNYGNS

SEQ ID NO: 62 - protein, 9 aa, synthetic construct
AGGYDSVVD

SEQ ID NO: 63 - protein, 8 aa, synthetic construct
GFSLSRYA

SEQ ID NO: 64 - protein, 8 aa, synthetic construct
GIDLSSYA

SEQ ID NO: 65 - protein, 8 aa, synthetic construct
GFTLSNNA

SEQ ID NO: 66 - protein, 9 aa, synthetic construct
GFSFSSSYY

SEQ ID NO: 67 - protein, 9 aa, synthetic construct
GFSFSSGYY

SEQ ID NO: 68 - protein, 8 aa, synthetic construct
GIDLSNTW

SEQ ID NO: 69 - protein, 8 aa, synthetic construct
SGFSLNNY

SEQ ID NO: 70 - protein, 8 aa, synthetic construct
GIDLSYYA

SEQ ID NO: 71 - protein, 9 aa, synthetic construct
TFSFNSRYW

SEQ ID NO: 72 - protein, 8 aa, synthetic construct
GFSLNRYA

SEQ ID NO: 73 - protein, 8 aa, synthetic construct
GFSLSDYA

SEQ ID NO: 74 - protein, 8 aa, synthetic construct
GFSLSSYG

SEQ ID NO: 75 - protein, 8 aa, synthetic construct
GFSLSDYA

SEQ ID NO: 76 - protein, 9 aa, synthetic construct
GFDLRSYYY

SEQ ID NO: 77 - protein, 9 aa, synthetic construct
GFDLSSNYY

SEQ ID NO: 78 - protein, 8 aa, synthetic construct
GFSLSSHD

SEQ ID NO: 79 - protein, 9 aa, synthetic construct
TFSFNSRYW

SEQ ID NO: 80 - protein, 8 aa, synthetic construct
GFSLSSYY

SEQ ID NO: 81 - protein, 9 aa, synthetic construct
GFDLSSYYY

SEQ ID NO: 82 - protein, 7 aa, synthetic construct
IGSSGLT

SEQ ID NO: 83 - protein, 7 aa, synthetic construct
INIGGGT

SEQ ID NO: 84 - protein, 7 aa, synthetic construct
IYASGRT

SEQ ID NO: 85 - protein, 8 aa, synthetic construct
IYASSGST

SEQ ID NO: 86 - protein, 9 aa, synthetic construct
IYTGRTDFT

SEQ ID NO: 87 - protein, 7 aa, synthetic construct
ITDSGTT

SEQ ID NO: 88 - protein, 7 aa, synthetic construct
IFNNGDI

SEQ ID NO: 89 - protein, 7 aa, synthetic construct
INGRGDT

SEQ ID NO: 90 - protein, 7 aa, synthetic construct
INNGDIS

SEQ ID NO: 91 - protein, 7 aa, synthetic construct
IGSSGST

SEQ ID NO: 92 - protein, 7 aa, synthetic construct
INSRDDT

SEQ ID NO: 93 - protein, 7 aa, synthetic construct
IYPSGSI

SEQ ID NO: 94 - protein, 7 aa, synthetic construct
INNGDIY

SEQ ID NO: 95 - protein, 8 aa, synthetic construct
IHGGEGNT

SEQ ID NO: 96 - protein, 8 aa, synthetic construct
IYSSNTRT

SEQ ID NO: 97 - protein, 7 aa, synthetic construct
IISSGNT

SEQ ID NO: 98 - protein, 7 aa, synthetic construct
INNGDIT

SEQ ID NO: 99 - protein, 7 aa, synthetic construct
ITTAGPL

SEQ ID NO: 100 - protein, 8 aa, synthetic construct
IYTSSGAT

SEQ ID NO: 101 - protein, 18 aa, synthetic construct
ARGMWYDDSDDYEDYFNL

SEQ ID NO: 102 - protein, 14 aa, synthetic construct
ARDVDAHTLTYFTL

SEQ ID NO: 103 - protein, 16 aa, synthetic construct
ARGDTETDYGIPYFDL

SEQ ID NO: 104 - protein, 14 aa, synthetic construct
AILGADYRLTRLDL

SEQ ID NO: 105 - protein, 17 aa, synthetic construct
ARGDYSGGVGGNYWLDL

SEQ ID NO: 106 - protein, 14 aa, synthetic construct
GRDPGDITSGTNDL

SEQ ID NO: 107 - protein, 11 aa, synthetic construct
ARTGYRTGGWL

SEQ ID NO: 108 - protein, 19 aa, synthetic construct
AREDSAIPFIVGNYYGMDL

SEQ ID NO: 109 - protein, 13 aa, synthetic construct
AKGGNLAGDCYGL

SEQ ID NO: 110 - protein, 22 aa, synthetic construct
ARDLDDSYGYTYATGMDIRLDL

SEQ ID NO: 111 - protein, 19 aa, synthetic construct
AREDSSIPFIVGNYYGMDL

SEQ ID NO: 112 - protein, 12 aa, synthetic construct
VRYLTGSSDLHL

SEQ ID NO: 113 - protein, 11 aa, synthetic construct
ARPGYRTGIWL

SEQ ID NO: 114 - protein, 8 aa, synthetic construct
RGGWTNYF

SEQ ID NO: 115 - protein, 8 aa, synthetic construct
RGGWTNYL

SEQ ID NO: 116 - protein, 10 aa, synthetic construct
ARDVYSGASP

SEQ ID NO: 117 - protein, 13 aa, synthetic construct
AKGGNLAGDCYGL

SEQ ID NO: 118 - protein, 15 aa, synthetic construct
ARGHAGSIYYSYFDL

SEQ ID NO: 119 - protein, 9 aa, synthetic construct
RGGWCDFNL

SEQ ID NO: 120 - protein, 110 aa, synthetic construct
ELVLTQTPASVSAAVGGTVTINCQASQNIYSNLAWYQQKPGQPPKLLIYGASNLESGVPSRFKGSGSGTQFTLTISDLECDDAATYYCQSGYYSSSTDIAFGGGTEVVVK

SEQ ID NO: 121 - protein, 110 aa, synthetic construct
ELVLTQTPASVSEPVGGTVTIKCQASQTISSWLSWYQQKPGQPPKLLIYYAFNLASGVPSRFKGSGSGTEFTLTISDLECADAATYYCQQGISSSNVDNVFGGGTEVVVK

SEQ ID NO: 122 - protein, 113 aa, synthetic construct
ELVLTQTPASVSEPVGGTVTIKCQASQTISSWLSWYQQKPGQPPKLLIYYAFNLASGVPSRFKGSGSGTEYTLTISDLECADAATYYCQCTYGSGSSSSYGCAFGGGTELEIK

SEQ ID NO: 123 - protein, 110 aa, synthetic construct
ELVMTQTPSPVSAAVGGTVTISCQSSQSVYSNYLSWYQQKPGQPPKLLIYETSTLASGVPSRFKGSGSGTQFTLTISDVQCDDAATYYCQGGYSEIIENTFGGGTEVEIK

SEQ ID NO: 124 - protein, 107 aa, synthetic construct
ELVMTQTPASVEAAVGGTVTIKCQASQSIGSVLAWYQQKPGQRPKLLISGVFDLASGVPSRFKGSGSGTEFTLTISDLECADAATYYCQYIPYGSSPFGGGTEVVVK

SEQ ID NO: 125 - protein, 111 aa, synthetic construct
ELVMTQTASPVSAAVGGTVTINCQASQSIGSTYLSWYQQKPGQPPKLLIYKASILASGVPSRFSGSGSGTEYTLTISGVQCDDAATYYCLYGGFGSSTGDAFGGGTVLVVK

SEQ ID NO: 126 - protein, 108 aa, synthetic construct
ELVLTQTPASVSEPVGGTVTIKCQASQTIYSNLAWYQQKPGQRPKLLIYQASKLASGVPSRFKGSGSGTEYTLTISDLECADAATYYCQSYYGADDYTFGGGTEVVVK

SEQ ID NO: 127 - protein, 111 aa, synthetic construct
ELVMTQTPSPVSAAVGGTVSISCQSSKSVYNNNALSWFQQKPGQPPKVLIYGVSTLDSGVSSRFSGSGYGTEFTLTISDVQCDDAATYYCAGDYSDISDNNFGGGTELEIK

SEQ ID NO: 128 - protein, 111 aa, synthetic construct
ELDMTQTPASVSEPVGGTVTIKCQASQSISSYLAWYQQKPGQPPKRLIFEASTLASGVPSRFSGSGSGTDFTLTISDLECADAATYYCQNNNGFSGSNFNNFGGGTEVEIK

SEQ ID NO: 129 - protein, 111 aa, synthetic construct
ELVMTQTPASVEVAVGGTVTIKCQASQTIYSSLAWYQQKPGQPPKLLIYKASTLASGVPSRFKGSGSGTQFTLTISGVQCDDAATYYCQQGSSISNVDKNAFGGGTEVEIK

SEQ ID NO: 130 - protein, 111 aa, synthetic construct
ELVLTQTPASVSEPVGGTVTIKCQASQSIGSYLSWYQQKAGQPPKRLIYEASTLASGVPSRFSGSGSGTDFTLTISDLECADVATYYCQNNNGMTVSDFNAFGGGTEVEIK

SEQ ID NO: 131 - protein, 112 aa, synthetic construct
ELDLTQTPASVSAAVGGTVTINCQSSQIIDHDHLSWYQQKPGQRPKLLIYRASTLTSGVPSRFKGSGSGTDFTLTISDLECADVATYYCQNNNGMTVSDFNAFGGGTEVEIK

SEQ ID NO: 132 - protein, 110 aa, synthetic construct
ELVLTQTPSSTSAAVGGTVTISCQSSQSVVDKNWLAWYQQKPGQPPKLLIYEASKLASGVPPRFSGSGSGTQFTLTISGVQCDDAATYYCAGDFESGVSGFGGGTEVEIK

SEQ ID NO: 133 - protein, 111 aa, synthetic construct
ELVLTQTPSPVSAAVGGTVTINCQSSKNIYNNNALSWFQQKPGQPPKLLIYGASTLASGVPSRFKGSGSGTQFTLTISDVQCDDAATYYCAADYSDISDNNFGGGTEVVVK

SEQ ID NO: 134 - protein, 109 aa, synthetic construct
ELVLTQTPSSVSAAVGGTVTISCQSSQSVYSNNYLAWYQQKPGQPPKLLIYAASTLASGVPSRFKGSGSGTQFTLTISGVQCDDAAVYYCLGGYNDDANFGGGTEVEIK

SEQ ID NO: 135 - protein, 109 aa, synthetic construct
ELDLTQTPSSVSAAVGGTVTISCQSSESVYSNNHLAWYQQKPGQPPKLLIYAASTLASGVPSRFSGSGSGTQFTLTISGVQCDDAAVYYCLGGYNDDANFGGGTEVVVK

SEQ ID NO: 136 - protein, 110 aa, synthetic construct
ELDLTQTPASVEAAVGGTVTIKCQASQSIDNNDLAWYQQKPGQPPNLLISRTSTLASGVSSRFKGSGSGTEFTLTISDLECADAATYYCQSYCVNTYGYTFGGGTEVVVK

SEQ ID NO: 137 - protein, 112 aa, synthetic construct
ELVMTQTPASVEAAVGGTVTIKCQASQSISNHLGWYQQKPGQPPKLLIYRASTLESGVSSRFKGSGSGSEFTLTISDLECADAATYYCQSYYIINRSNYANSFGGGTEVEIK

SEQ ID NO: 138 - protein, 112 aa, synthetic construct
ELVMTQTPASVEAAVGGTVTIKCQASESINSWLAWYQQKPGQRPKLLIYDASKLASGVPSRFKGSGSGTQFTLTISDLECADAATYYCQSYYIINRSNYGNSFGGGTEVEIK

SEQ ID NO: 139 - protein, 109 aa, synthetic construct
ELDLTQTPASVSAAVGGTVTISCQSSENLYKDNYLAWYQQKPGQPPKLLIYGASNLASGVPSRFKGSGSGTQFTLTISDLECDDAATYYCAGGYDSVVDFGGGTEVVVK

SEQ ID NO: 140 - protein, 121 aa, synthetic construct
QSVEESGGRLVTPGTPLTLTCTVSGFSLSRYAMTWVRQAPGKGLEWIGIIGSSGLTYFATWAKGRFTISKTSTTVDLKITSPTTEDTATYFCARGMWYDDSDDYEDYFNLWGPGTLVTISS

SEQ ID NO: 141 - protein, 117 aa, synthetic construct
QSVKESGGRLVTPGTPLTLTCTVSGIDLSSYAMSWVRQAPGKGLEWIGTINIGGGTWDATWARGRFTISRTSTTVDLKITSPTIGDTATYFCARDVDAHTLTYFTLWGPGTLVTISS

SEQ ID NO: 142 - protein, 119 aa, synthetic construct
QSVKESGGRLVTPGTPLTLTCTVSGFTLSNNAISWVRQAPGKGLEWIGIIYASGRTYYATWAKGRFTISKTSTTVDLKMTSPTTEDTATYFCARGDTETDYGIPYFDLWGPGTLVTISS

SEQ ID NO: 143 - protein, 122 aa, synthetic construct
SQSLKESGGDLVKPGASRTLTCIAPGFSFSSSYYMCWVRQAPGKGLEWIACIYASSGSTYYASWAKGRFTISKTSSTTVTLQMTTLTAADTATYFCAAILGADYRLTRLDLWGQGTLVTVSS

SEQ ID NO: 144 - protein, 118 aa, synthetic construct
QSLEESGGRLVTPGTPLTLTCTASGFSLNRYYMLWVRQAPGEGLEWIGTISYGDTTYYASWAKGRFTISKTSTTVDLKMTSPTTEDTATYFCARADTGDNGYLGLQLWGPGTLVTVSS

SEQ ID NO: 145 - protein, 124 aa, synthetic construct
QSLEESGGDLVKPGASLTLTCTASGFSFSSGYYMCWVRQAPGKGLEWIACIYTGRTDFTDYASWAKGRFTISKTSSTTVTLQLTTLTAADTATYFCARGDYSGGVGGNYWLDLWGQGTLVTISS

SEQ ID NO: 146 - protein, 117 aa, synthetic construct
QSLEESGGRLVTPGTPLTLTCTVSGIDLSNTWMNWVRQAPGKGLEWIGVITDSGTTYYANWAKGRFTISRTSTTVDLKMPSLTTEDTATYFCGRDPGDITSGTNDLWGPGTLVTISS

SEQ ID NO: 147 - protein, 114 aa, synthetic construct
EQSVEESGGRLVTPGGSLTLTCTASGFSLNNYAIIWVRQAPGKGLEYIGIFNNGDIYYANWAKGRFTISKTSTTVGLKIVSPTTEDTATYFCARTGYRTGGWLWGPGTLVTISS

SEQ ID NO: 148 - protein, 122 aa, synthetic construct
QSVKESGGRLVTPGTPLTLTCTVSGIDLSYYAMSWVRQAPGKGLEYIGIINGRGDTGYATWAKGRFTISKTSTTVDLRITSPTIEDTATYFCAREDSAIPFIVGNYYGMDLWGPGTLVTVSS

SEQ ID NO: 149 - protein, 120 aa, synthetic construct
SQSLEESGGDLVKPGASLTLTCTASTFSFNSRYWTCWVRQAPGKGLEWIGCINNGDISTYYASWATGRFTISKSSSTTVTLHMTSLTAADTATYFCAKGGNLAGDCYGLWGPGTLVTISS

SEQ ID NO: 150 - protein, 125 aa, synthetic construct
SSVEESGGRLVAPGTPLTLTCTVSGFSLNRYAMSWVRQAPGKGLEWIGIIGSSGSTYYASWAKGRFTISKTSTTVDLKITSPTTEDTATYFCARDLDDSYGYTYATGMDIRLDLWGQGTLVTVSS

SEQ ID NO: 151 - protein, 123 aa, synthetic construct
QSVKESGGGLFKPMDTLTLTCTVSGFSLSDYAMSWVRQAPGKGLEWIGIINSRDDTGYASWAKGRFTISKTSSTTVDLRITSPTTEDTATYFCAREDSSIPFIVGNYYGMDLWGPGTLVTVSS

SEQ ID NO: 152 - protein, 115 aa, synthetic construct
QSLEESGGRLVTPGTPLTLTCTVSGFSLSSYGVHWVRQAPGKGLDWIGKIYPSGSIYYSSWAKGRFTISKTSTTVDLKMTSLTTEDTATYFCVRYLTGSSDLHLWGPGTLVTISS

SEQ ID NO: 153 - protein, 114 aa, synthetic construct
QSVKESGGRLVTPGGSLTLTCTVSGFSLSDYAMIWVRQAPGKGLEYIGIINNGDIYYATWAKGRFTISETSSTTMGLNIISPTTEDTATYFCARPGYRTGIWLWGPGTLVTISS

SEQ ID NO: 154 - protein, 116 aa, synthetic construct
SQSVKESGGDLVKPGASLTLTCKASGFDLRSYYYMCWVRQAPGKGLEWIACIHGGEGNTYYASWAKGRFTISKTSSTAVTLQMTSLTAADTATYFCARGGWTNYFWGPGTLVTVSS

SEQ ID NO: 155 - protein, 116 aa, synthetic construct
EQSLKESGGDLVKPGASLTLTCTASGFDLSSNYYMCWVRQAPGKGPEWIACIYSSNTRTWYARWAKGRFTISKTSSTAVTLQMTSLTAADTATYFCARGGWTNYLWGPGTLVTISS

SEQ ID NO: 156 - protein, 113 aa, synthetic construct
QSVEESGGRLVTPGTPLTLTCTVSGFSLSSHDMIWVRQAAGKGLEWIGLIISSGNTWYASWAKGRFTISKTSTTVDLKMTSLTTEDTATYFCARDVYSGASPWGPGTLVTISS

SEQ ID NO: 157 - protein, 118 aa, synthetic construct
QSVKSGGGLVKPGASLTLTCKASTFSFNSRYWTCWVRQAPGKGLEWIGCINNGDITTYYTNWATGRFTISKSSSTTVTLQMTSLTAADTATYFCAKGGNLAGDCYGLWGPGTLVTISG

SEQ ID NO: 158 - protein, 118 aa, synthetic construct
QSLEESGGRLVTPGTPLTLTCTASGFSLSSYYMSWVRQAPGEGLEWIGTITTAGPLYYATWAKGRFTISKTSTTVDLKMTGPTTEDTATYFCARGHAGSIYYSYFDLWGPGTLVTVSS

SEQ ID NO: 159 - protein, 116 aa, synthetic construct
QSVKESGGGLVKPEGSLTLTCKASGFDLSSYYYMCWVRQAPGKGLEWIACIYTSSGATWYANWAKGRFTISKTSSTTVTLQMTALTAADTATYFCARGGWCDFNLWGPGTLVTISS

SEQ ID NO: 160 - skipped

SEQ ID NO: 161 - protein, 214 aa, synthetic construct
DIQMTQSPSSVSASVGDRVTITCQASQSIGSVLAWYQQKPGKAPKLLISGVFDLASGVPSRFSGSGSGTDFTLTISSLQPEDFATYYCQYIPYGSSPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 451 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 451 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 216 aa, synthetic construct
ELVMTQTPASVEAAVGGTVTIKCQASETISSRLAWYQQKPGQPPKLLIYQASKLPSGVPSRFKGTGSGTEYTLTISDLECADAATYYCQGCYYGGGSFYDSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 165 - protein, 439 aa, synthetic construct
QSVEESGGRLVTPGGSLTLTCTVSGFSLSSYDMSWVRQAPGKGLEWIGISWNSGFVDYASWAKGRFSISKTSTTVDLKITSPTTEDTATYFCARLGADDIYYFNLWGPGTLVTISSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2025/0369982 A1
(43) Pub. Date: Dec. 4, 2025

(54) METHODS OF DETECTING DDR1 PHOSPHORYLATION

(71) Applicant: IncendiaTherapeutics, Inc., Cambridge, MA (US)

(72) Inventors: Glenn BEGLEY, Cambridge, MA (US); Thomas SCHUERPF, Cambridge, MA (US); Laurent AUDOLY, Cambridge, MA (US)

(21) Appl. No.: 18/875,678

(22) PCT Filed: Jun. 16, 2023
(86) PCT No.: PCT/US2023/068603

§ 371 Date: Dec. 16, 2024

Related U.S. Application Data
(60) Provisional application No. 63/366,567, filed on Jun. 17, 2022.

(51) Int. Cl. G01N 33/68; C07K 16/28; G01N 33/50; G01N 33/574

(52) U.S. Cl. CPC G01N 33/6857; C07K 16/2851; G01N 33/5017; G01N 33/5044; G01N 33/57492; G01N 33/6893; C07K 2317/565; G01N 2333/70503; G01N 2440/14

(57) ABSTRACT

The instant disclosure provides methods of detecting discoidin domain receptor tyrosine kinase 1 (DDR1) phosphorylation to determine the effectiveness or likely effectiveness of DDR1 antagonistic therapies. The disclosure provides antibodies for use in the treatment of DDR1 related disorders that specifically bind to DDR1. Also provided herein are compositions comprising these antibodies, nucleic acids encoding these antibodies, expression vectors and host cells for making these antibodies, for detecting phosphorylated DDR1.

### 1. CROSS-REFERENCES TO RELATED APPLICATIONS

This application claims benefit of priority to U.S. Provisional Application Ser. No. 63/366,567, filed Jun. 17, 2022, the entire contents of which are hereby incorporated by reference.

### 2. REFERENCE TO A SEQUENCE LISTING

This application contains a sequence listing which has been submitted electronically in ST.26 format and is hereby incorporated by reference in its entirety (said ST.26 copy, created on Jun. 16, 2023, is named “201000_seqlist.xml” and is 151,659 bytes in size).

### 3. FIELD

The instant disclosure relates to methods of detecting discoidin domain receptor tyrosine kinase 1 (DDR1) phosphorylation to determine the effectiveness or likely effectiveness of DDR1 inhibitors, such as anti-DDR1 antibodies, or methods of screening for such DDR1 inhibitors.

### 4. BACKGROUND

Receptor tyrosine kinases (RTKs) play a key role in the communication of cells with their microenvironment. These molecules are involved in the regulation of cell growth, differentiation and metabolism. The DDR1 protein encoded by the DDR1 gene is an RTK that is widely expressed in normal and transformed epithelial cells and is activated by various types of collagen. The DDR1 protein belongs to a subfamily of tyrosine kinase receptors with a homology region to the Dictyostelium discoideum protein discoidin I in their extracellular domain. Its autophosphorylation is achieved by all collagens so far tested (type I to type VI). In situ studies and Northern-blot analysis showed that expression of DDR1 encoded protein is restricted to epithelial cells, particularly in the kidney, lung, gastrointestinal tract, and brain. In addition, the DDR1 protein is significantly over-expressed in several human tumors from breast, ovarian, esophageal, and pediatric brain. In addition to expression in cancers, DDR1 protein is also expressed in the kidney, lung, gastrointestinal tract, skin, and brain, among other organs and has been implicated in fibrosis of the skin, lung and liver.

Accordingly, methods of determining the effectiveness or likely effectiveness of DDR1 inhibitors in the treatment or prevention DDR1 related disorders would be highly desirable.

### 5. SUMMARY

The instant disclosure herein demonstrates that anti-DDR1 antibodies are capable of inhibiting DDR1 phosphorylation. DDR1 related disorders are associated with increased collagen-mediated DDR1 phosphorylation and subsequent downstream signaling. Thus, methods of detecting DDR1 phosphorylation may be used to determine the effectiveness of or the likely effectiveness of anti-DDR1 antibodies in the treatment of DDR1 related disorders.

An anti-DDR1 antibody may be deemed effective for treating a DDR1 related disorder if it is capable of inhibiting DDR1 phosphorylation and/or the ability of DDR1 to interact with collagen. Moreover, an anti-DDR1 antibody is likely to be effective for treating a DDR1 related disorder if it is capable of inhibiting DDR1 phosphorylation and/or the ability of DDR1 to interact with collagen.

In one aspect, the instant disclosure provides a method of monitoring the effectiveness of an anti-discoidin domain receptor tyrosine kinase 1 (DDR1) antibody or antigen-binding fragment thereof in a subject in need thereof comprising: a) administering an effective amount of the anti-DDR1 antibody to the subject; and b) detecting a level of DDR1 phosphorylation in a sample from the subject, wherein a decrease in DDR1 phosphorylation in the sample from the subject in comparison to a positive reference sample indicates that the administration of the anti-DDR1 antibody is effective.

In some embodiments, the subject has cancer. In some embodiments, the cancer is selected from the group consisting of pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

In some embodiments, the subject has a fibrotic condition. In some embodiments, the fibrotic condition is selected from the group consisting of: skin hypertrophic scarring, scleroderma, lung scarring, idiopathic pulmonary fibrosis, cirrhotic liver fibrosis, renal fibrosis, and interstitial lung disease.

In one aspect, the instant disclosure provides a method of treating a DDR1 related disorder in a subject in need thereof comprising: a) administering an effective amount of an anti-DDR1 antibody or antigen-binding fragment thereof to the subject; and b) detecting a level of DDR1 phosphorylation in a sample from the subject, wherein a decrease in DDR1 phosphorylation in the sample from the subject in comparison to a positive reference sample indicates that the treatment is effective.

In one aspect, the instant disclosure provides a method of screening for a subject with a DDR1 related disorder that is likely to be effectively treated with an anti-DDR1 antibody comprising detecting a level of DDR1 phosphorylation in a sample from the subject, wherein if DDR1 phosphorylation in the sample from the subject is higher in comparison to a negative reference sample, then the DDR1 related disorder is likely to be effectively treated with an anti-DDR1 antibody.

In one aspect, the instant disclosure provides a method of treating a DDR1 related disorder in a subject in need thereof comprising: a) detecting a level of DDR1 phosphorylation in a sample from the subject; and b) administering an effective amount of an anti-DDR1 antibody or antigen-binding fragment thereof to the subject if DDR1 phosphorylation in the sample from the subject is higher in comparison to a negative reference sample.

In some embodiments, the DDR1 related disorder is cancer. In some embodiments, the cancer is selected from the group consisting of: pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

In some embodiments, the DDR1 related disorder is a fibrotic condition. In some embodiments, the fibrotic condition is selected from the group consisting of: skin hypertrophic scarring, scleroderma, lung scarring, idiopathic pulmonary fibrosis, cirrhotic liver fibrosis, renal fibrosis, and interstitial lung disease.

In some embodiments, the sample from the subject described herein comprises tumor tissue. In some embodiments, the sample from the subject described herein comprises one or more selected from the group consisting of: blood cells, skin tissue, lung tissue, renal tissue, and liver tissue. In some embodiments, the sample from the subject described herein comprises a skin punch biopsy sample.

In one aspect, the instant disclosure provides a method of screening for an anti-DDR1 antibody or antigen-binding fragment thereof that is effective in treating a DDR1 related disorder comprising: a) administering an effective amount of the anti-DDR1 antibody or antigen-binding fragment thereof to a cell; and b) detecting a level of DDR1 phosphorylation in the cell, wherein a decrease in DDR1 phosphorylation in the cell in comparison to a positive reference cell indicates that the anti-DDR1 antibody or antigen-binding fragment thereof is effective in treating the DDR1 related disorder.

In one aspect, the instant disclosure provides a method of screening for an anti-DDR1 antibody or antigen-binding fragment thereof that is effective in reducing collagen interaction with a cell comprising: a) administering an effective amount of the anti-DDR1 antibody or antigen-binding fragment thereof to the cell; and b) detecting a level of DDR1 phosphorylation in the cell, wherein a decrease in DDR1 phosphorylation in the cell in comparison to a positive reference cell indicates that the anti-DDR1 antibody or antigen-binding fragment thereof is effective in reducing collagen interaction with the cell.

In some embodiments, the cell as described herein is a cancer cell. In some embodiments, the cancer cell is derived from a cancer selected from the group consisting of: pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

In some embodiments, the cell as described herein is one or more selected from the group consisting of: a skin cell, a lung cell, a kidney cell, and a liver cell.

In some embodiments, the anti-DDR1 antibody or antigen-binding fragment thereof described herein comprises a heavy chain variable domain (VH) comprising the CDRH1, CDRH2 and CDRH3 amino acid sequences of the VH amino acid sequence of SEQ ID NO: 4 or 13 and a light chain variable domain (VL) comprising the CDRL1, CDRL2 and CDRL3 amino acid sequences of the VL amino acid sequence of SEQ ID NO: 3, 11, or 12.

In some embodiments, a) the CDRL1 comprises the amino acid sequence of: SEQ ID NO: 5; b) the CDRL2 comprises the amino acid sequence QAS; c) the CDRL3 comprises the amino acid sequence of SEQ ID NO: 7; d) the CDRH1 comprises the amino acid sequence of SEQ ID NO: 8; e) the CDRH2 comprises the amino acid sequence of SEQ ID NO: 9; and f) the CDRH3 comprises the amino acid sequence of SEQ ID NO: 10.

In some embodiments, a) the CDRL1 comprises the amino acid sequence of SEQ ID NO: 17; b) the CDRL2 comprises the amino acid sequence GVF; c) the CDRL3 comprises the amino acid sequence of SEQ ID NO: 19; d) the CDRH1 comprises the amino acid sequence of SEQ ID NO: 20; e) the CDRH2 comprises the amino acid sequence of SEQ ID NO: 21; and f) the CDRH3 comprises the amino acid sequence of SEQ ID NO: 22.

In some embodiments, the anti-DDR1 antibody comprises a) a VL domain comprising an amino acid sequence that is at least 90% identical to an amino acid sequence selected from the group consisting of SEQ ID NOs: 3, 11, and 12; and b) a VH domain comprising an amino acid sequence that is at least 90% identical to an amino acid sequence selected from the group consisting of SEQ ID NOs: 4 and 13.

In some embodiments, the anti-DDR1 antibody comprises a) a VL domain comprising an amino acid sequence selected from the group consisting of SEQ ID NOs: 3, 11, and 12; and b) a VH domain comprising an amino acid sequence selected from the group consisting of SEQ ID NOs: 4 and 13.

In some embodiments, the anti-DDR1 antibody comprises a VL domain and a VH domain selected from the group consisting of: a) SEQ ID NOs: 3 and 4, respectively; b) SEQ ID NOs: 11 and 13, respectively; and c) SEQ ID NOs: 12 and 13, respectively.

In some embodiments, the anti-DDR1 antibody comprises a VL domain and a VH domain comprising the amino acid sequence of SEQ ID NOs: 3 and 4, respectively. In some embodiments, the anti-DDR1 antibody comprises a VL domain and a VH domain comprising the amino acid sequence of SEQ ID NOs: 11 and 13, respectively. In some embodiments, the anti-DDR1 antibody comprises a VL domain and a VH domain comprising the amino acid sequence of SEQ ID NOs: 12 and 13, respectively.

In some embodiments, detecting a level of DDR1 phosphorylation comprises detecting the level of phosphorylation of a cleaved form of DDR1. In some embodiments, the cleaved form of DDR1 has a molecular weight of approximately 65 kDa.

### 6. BRIEF DESCRIPTION OF THE DRAWINGS

FIGS. 1A through 1D demonstrate 9H1-WT monoclonal antibody-mediated inhibition of collagen I-induced discoidin domain receptor tyrosine kinase 1 (DDR1) phosphorylation. FIG. 1A depicts phosphorylated DDR1 normalized to vinculin levels in cancer cells pretreated with increasing concentrations of 9H1-WT (or IgG1-WT as a negative control) and stimulated with human collagen I. FIG. 1B depicts phosphorylated DDR1 normalized to vinculin levels in cancer cells pretreated with increasing concentrations of 9H1-WT (or IgG1-WT as a negative control) and stimulated with rat collagen I. FIGS. 1C-1D depict representative immunoblots corresponding to the quantified results described in FIGS. 1A-1B.

FIGS. 2A through 2J demonstrate that 9H1-WT and Ab #33 monoclonal antibodies have no impact on cancer cell proliferation or cell death. FIG. 2A depicts representative fluorescence images of T47D cells treated with 9H1-WT or control IgG1-WT. FIG. 2B depicts representative fluorescence images of T47D cells treated with Ab #33 or control inert IgG1. FIGS. 2C-2F are time-course graphs of nuclei count (FIG. 2C) and total cell area (FIG. 2D) as measures of cell proliferation and annexin V-positive (FIG. 2E) and Cytox Green-positive (FIG. 2F) cells as measures of cell death in response to 9H1-WT, IgG1-WT as a negative control and paclitaxel as a positive control. FIGS. 2G-2J are time-course graphs of nuclei count (FIG. 2G) and total cell area (FIG. 2H) as measures of cell proliferation and Annexin V-positive (FIG. 2I) and Cytox Green-positive (FIG. 2J) cells as measures of cell death in response to Ab #33, inert IgG1 as a negative control and paclitaxel as a positive control.

FIGS. 3A and 3B demonstrate that collagen I and V efficiently induce phosphorylation of DDR1. FIG. 3A depicts a representative immunoblot (left) and quantification (right) of DDR1 phosphorylation (pDDR1) in response to stimulation with 25 μg/ml of various types of rat and human collagen. FIG. 3B depicts a representative immunoblot (left) and quantification (right) of pDDR1 in response to stimulation with 50 μg/ml of the types of rat and human collagen used in FIG. 3A.

FIGS. 4A through 4E demonstrate that anti-DDR1 monoclonal antibodies reach in vivo levels that are sufficient to bind DDR1 and inhibit collagen I-induced phosphorylation. FIG. 4A depicts a schematic of the study outline. FIGS. 4B-4D depict in vivo antibody concentrations over the course of 168 hours following administration of humanized mAb #9H1 with inert IgG1Fc (FIG. 4B), humanized mAb #9H1 with wt IgG1Fc (FIG. 4C), and chimeric rabbit/human mAb #33 with inert IgG1 Fc (FIG. 4D). FIG. 4E depicts the mean antibody concentration over the course of 168 hours for each of the antibodies described in FIGS. 4B-4D.

FIGS. 5A and 5B demonstrate inhibition of collagen I- and V-induced phosphorylation of DDR1 by 9H1-WT mAb. FIG. 5A depicts pDDR1 normalized to vinculin in T47D cells pretreated with 9H1-WT, negative control IgG1 or positive control 2.45-IN followed by stimulation with 50 μg/ml of human collagen I or collagen V. FIG. 5B depicts total DDR1 normalized to vinculin in the pretreated and stimulated cells of FIG. 5A.

FIGS. 6A through 6D demonstrate the calculated IC50 for 9H1-WT (PRTH-101) inhibition of DDR1 phosphorylation. FIGS. 6A-6C depict inhibition curves for three independent experiments and their calculated IC50 values+/−standard error of the mean. FIG. 6D depicts a combined curve for all three experiments and the calculated IC50 value+/−standard error of the mean.

FIGS. 7A through 7C demonstrate 9H1-WT inhibition of DDR1-expressing cell adhesion to human collagen I. FIG. 7A depicts representative fluorescence images of WT or DDR1 overexpressing HEK293 cells treated with increasing concentrations of 9H1-WT (PRTH-101) or control IgG1-WT and incubated on collagen I-coated adhesion plates. FIGS. 7B-C depict the number of adherent cells for the experimental groups described in FIG. 7A in two independent experiments.

FIG. 8 demonstrates the calculated IC50 for 9H1-WT (PRTH-101) inhibition of DDR1-expressing cell adhesion. The inhibition curve comprising the merged values of four independent experiments depicts the number of adherent cells at increasing concentrations of 9H1-WT (PRTH-101).

FIGS. 9A and 9B demonstrate 9H1-WT inhibition of collagen II- and collagen III-mediated phosphorylation of DDR1 in T47D cells. FIG. 9A depicts phosphorylated DDR1 normalized to vinculin expression in T47D cells pretreated with increasing concentrations of 9H1-WT (PRTH-101) or control IgG1-WT and stimulated with 50 μg/ml human collagen II or collagen III. FIG. 9B depicts an experiment identical to that depicted in FIG. 9A, but further including a 50 μg/ml human collagen I stimulated experimental group for comparison.

FIGS. 10A and 10B demonstrate rabbit mAb #33- and chimeric mAb #33-mediated inhibition of collagen I-induced DDR1 phosphorylation. FIG. 10A depicts phosphorylated DDR1 normalized to vinculin expression in T47D cells pretreated with increasing concentrations of rabbit mAb #33, control IgG57, 9H1-WT (PRTH-101), or control IgG1-WT and stimulated with 50 μg/ml human collagen I. FIG. 10B depicts phosphorylated DDR1 normalized to vinculin expression in T47D cells pretreated with increasing concentrations of chimeric mAb #33 or IgG Inert Fc and in the presence or absence of stimulation with 50 μg/ml human collagen I.

FIGS. 11A and 11B demonstrate that full-length and cleaved forms of DDR1 can be detected in skin samples from healthy human donors via western blot, but that only the cleaved form is phosphorylated. FIG. 11A depicts a western blot using a pDDR1-specific antibody on skin sample lysates prepared via various methods from 2 healthy human subjects. Also included are collagen-stimulated and unstimulated T47D breast cancer cells. Full-length pDDR1 (approximately 125 kDa) is indicated by a black arrow. Cleaved pDDR1 (approximately 60 kDa) is indicated by a white arrow. FIG. 11B depicts a western blot using a pDDR1-specific antibody and a general DDR1 antibody on skin sample lysates. The antibody used is indicated along the bottom. A representative general DDR1 antibody lane is depicted by a black arrow. A representative pDDR1-specific lane is depicted by a white arrow.

### 7. DETAILED DESCRIPTION

The instant disclosure provides methods of detecting DDR1 phosphorylation for use in monitoring the effectiveness or likely effectiveness of a DDR1 inhibitor (e.g., an anti-DDR1 antibody) to inhibit DDR1-mediated collagen interactions and/or to treat a DDR1 related disorder.

### 7.1 Definitions

As used herein, the term “DDR1” refers to Discoidin Domain Receptor Tyrosine Kinase 1 encoded by the DDR1 gene. Unless otherwise stated, the term “DDR1” refers to a DDR1 protein encoded by a wild-type DDR1 gene (e.g., GenBank™ accession number NM_013993.3). Exemplary DNA and amino acid sequences for human DDR1 are provided in Table 1 below. “DDR1 phosphorylation,” as used herein, refers to the attachment of a phosphoryl group to any residue of DDR1. For example, phosphorylation can occur on serine, threonine, or tyrosine residues of a DDR1 protein and can occur via intermolecular interactions (e.g., via a separate kinase) or intramolecular interactions (e.g., autophosphorylation). Exemplary DDR1 phosphorylation sites include, but are not limited to, Y484, Y513, Y520, S631, Y740, Y792, Y796, and Y797 relative to the amino acid sequence of SEQ ID NO: 2 shown in Table 1 below.

TABLE 1
Exemplary human DDR1 DNA and amino acid sequences.

| | |
|---|---|
| DNA | gctcctctccccggaacaggcccccgacagctgctctcgggagccgcctcccgacaccc |
| (SEQ ID | gagccccgccggcgcctcccgctcccggctcccggctcctggctccctccgcctccccc |
| NO: 1) | gcccctcgccccgccgccgaagaggccccgctcccgggtcggacgcctgggtctgccgg |
| | gaagagcgatgagaggtgtctgaaggtggctattcactgagcgatggggttggacttga |
| | aggaatgccaagagatgctgcccccacccccttaggcccgagggatcaggagctatggg |
| | accagaggccctgtcatctttactgctgctgctcttggtggcaagtggagatgctgaca |
| | tgaagggacattttgatcctgccaagtgccgctatgccctgggcatgcaggaccggacc |
| | atcccagacagtgacatctctgcttccagctcctggtcagattccactgccgcccgcca |
| | cagcaggttggagagcagtgacggggatggggcctggtgccccgcagggtcggtgtttc |
| | ccaaggaggaggagtacttgcaggtggatctacaacgactgcacctggtggctctggtg |
| | ggcacccagggacggcatgccgggggcctgggcaaggagttctcccggagctaccggct |
| | gcgttactcccgggatggtcgccgctggatgggctggaaggaccgctggggtcaggagg |
| | tgatctcaggcaatgaggaccctgagggagtggtgctgaaggaccttgggccccccatg |
| | gttgcccgactggttcgcttctacccccgggctgaccgggtcatgagcgtctgtctgcg |
| | ggtagagctctatggctgcctctggagggatggactcctgtcttacaccgcccctgtgg |
| | ggcagacaatgtatttatctgaggccgtgtacctcaacgactccacctatgacggacat |
| | accgtgggcggactgcagtatgggggtctgggccagctggcagatggtgtggtggggct |
| | ggatgactttaggaagagtcaggagctgcgggtctggccaggctatgactatgtgggat |
| | ggagcaaccacagcttctccagtggctatgtggagatggagtttgagtttgaccggctg |
| | agggccttccaggctatgcaggtccactgtaacaacatgcacacgctgggagcccgtct |
| | gcctggcggggtggaatgtcgcttccggcgtggccctgccatggcctgggagggggagc |
| | ccatgcgccacaacctagggggcaacctgggggaccccagagcccgggctgtctcagtg |
| | ccccttggcggccgtgtggctcgctttctgcagtgccgcttcctctttgcggggccctg |
| | gttactcttcagcgaaatctccttcatctctgatgtggtgaacaattcctctccggcac |
| | tgggaggcaccttcccgccagccccctggtggccgcctggcccacctcccaccaacttc |
| | agcagcttggagctggagcccagaggccagcagcccgtggccaaggccgaggggagccc |
| | gaccgccatcctcatcggctgcctggtggccatcatcctgctcctgctgctcatcattg |
| | ccctcatgctctggcggctgcactggcgcaggctcctcagcaaggctgaacggagggtg |
| | ttggaagaggagctgacggttcacctctctgtccctggggacactatcctcatcaacaa |
| | ccgcccaggtcctagagagccacccccgtaccaggagccccggcctcgtgggaatccgc |
| | cccactccgctccctgtgtccccaatggctctgcgttgctgctctccaatccagcctac |
| | cgcctccttctggccacttacgcccgtccccctcgaggcccgggcccccccacacccgc |
| | ctgggccaaacccaccaacacccaggcctacagtggggactatatggagcctgagaagc |
| | caggcgccccgcttctgcccccacctccccagaacagcgtcccccattatgccgaggct |
| | gacattgttaccctgcagggcgtcaccgggggcaacacctatgctgtgcctgcactgcc |
| | cccaggggcagtcggggatgggccccccagagtggatttccctcgatctcgactccgct |
| | tcaaggagaagcttggcgagggccagtttggggaggtgcacctgtgtgaggtcgacagc |
| | cctcaagatctggttagtcttgatttcccccttaatgtgcgtaagggacaccctttgct |
| | ggtagctgtcaagatcttacggccagatgccaccaagaatgccaggaatgatttcctga |
| | aagaggtgaagatcatgtcgaggctcaaggacccaaacatcattcggctgctgggcgtg |
| | tgtgtgcaggacgaccccctctgcatgattactgactacatggagaacggcgacctcaa |
| | ccagttcctcagtgcccaccagctggaggacaaggcagccgagggggcccctggggacg |
| | ggcaggctgcgcaggggcccaccatcagctacccaatgctgctgcatgtggcagcccag |
| | atcgcctccggcatgcgctatctggccacactcaactttgtacatcgggacctggccac |
| | gcggaactgcctagttggggaaaatttcaccatcaaaatcgcagactttggcatgagcc |
| | ggaacctctatgctggggactattaccgtgtgcagggccgggcagtgctgcccatccgc |
| | tggatggcctgggagtgcatcctcatggggaagttcacgactgcgagtgacgtgtgggc |
| | ctttggtgtgaccctgtgggaggtgctgatgctctgtagggcccagccctttgggcagc |
| | tcaccgacgagcaggtcatcgagaacgcgggggagttcttccgggaccagggccggcag |
| | gtgtacctgtcccggccgcctgcctgcccgcagggcctatatgagctgatgcttcggtg |
| | ctggagccgggagtctgagcagcgaccacccttttcccagctgcatcggttcctggcag |
| | aggatgcactcaacacggtgtgaatcacacatccagctgcccctccctcagggagcgat |
| | ccaggggaagccagtgacactaaaacaagaggacacaatggcacctctgcccttcccct |
| | cccgacagcccatcacctctaatagaggcagtgagactgcaggtgggctgggcccaccc |
| | agggagctgatgccccttctccccttcctggacacactctcatgtccccttcctgttct |
| | tccttcctagaagcccctgtcgcccacccagctggtcctgtggatgggatcctctccac |
| | cctcctctagccatcccttggggaagggtggggagaaatataggatagacactggacat |
| | ggcccattggagcacctgggccccactggacaacactgattcctggagaggtggctgcg |
| | cccccagcttctctctccctgtcacacactggaccccactggctgagaatctgggggtg |
| | aggaggacaagaaggagaggaaaatgtttccttgtgcctgctcctgtacttgtcctcag |
| | cttgggcttcttcctcctccatcacctgaaacactggacctgggggtagccccgcccca |
| | gccctcagtcacccccacttcccacttgcagtcttgtagctagaacttctctaagccta |
| | tacgtttctgtggagtaaatattgggattggggggaaagagggagcaacggcccatagc |
| | cttggggttggacatctctagtgtagctgccacattgatttttctataatcacttgggg |
| | tttgtacatttttggggggagagacacagatttttacactaatatatggacctagcttg |
| | aggcaattttaatcccctgcactaggcaggtaataataaaggttgagttttccacaa |
| Amino | MGPEALSSLLLLLLVASGDADMKGHEDPAKCRYALGMQDRTIPDSDISASSSWSDSTAA |
| Acid | RHSRLESSDGDGAWCPAGSVFPKEEEYLQVDLQRLHLVALVGTQGRHAGGLGKEFSRSY |
| (SEQ ID | RLRYSRDGRRWMGWKDRWGQEVISGNEDPEGVVLKDLGPPMVARLVRFYPRADRVMSVC |
| NO: 2) | LRVELYGCLWRDGLLSYTAPVGQTMYLSEAVYLNDSTYDGHTVGGLQYGGLGQLADGVV |
| | GLDDERKSQELRVWPGYDYVGWSNHSFSSGYVEMEFEFDRLRAFQAMQVHCNNMHTLGA |
| | RLPGGVECRFRRGPAMAWEGEPMRHNLGGNLGDPRARAVSVPLGGRVARFLQCRELFAG |
| | PWLLFSEISFISDVVNNSSPALGGTFPPAPWWPPGPPPTNESSLELEPRGQQPVAKAEG |
| | SPTAILIGCLVAIILLLLLIIALMLWRLHWRRLLSKAERRVLEEELTVHLSVPGDTILI |
| | NNRPGPREPPPYQEPRPRGNPPHSAPCVPNGSALLLSNPAYRLLLATYARPPRGPGPPT |
| | PAWAKPTNTQAYSGDYMEPEKPGAPLLPPPPQNSVPHYAEADIVTLQGVTGGNTYAVPA |
| | LPPGAVGDGPPRVDEPRSRLREKEKLGEGQFGEVHLCEVDSPQDLVSLDFPLNVRKGHP |
| | LLVAVKILRPDATKNARNDELKEVKIMSRLKDPNIIRLLGVCVQDDPLCMITDYMENGD |
| | LNQFLSAHQLEDKAAEGAPGDGQAAQGPTISYPMLLHVAAQIASGMRYLATLNFVHRDL |
| | ATRNCLVGENFTIKIADFGMSRNLYAGDYYRVQGRAVLPIRWMAWECILMGKFTTASDV |
| | WAFGVTLWEVLMLCRAQPFGQLTDEQVIENAGEFFRDQGRQVYLSRPPACPQGLYELML |
| | RCWSRESEQRPPFSQLHRFLAEDALNTV |

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human and/or mouse DDR1). CDRH1, CDRH2 and CDRH3 denote the heavy chain CDRs, and CDRL1, CDRL2 and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent e.g., murine or lagomorph, e.g., rabbit CDRs and human framework regions (FRs). In an embodiment, the variable region is a primate (e.g., non-human primate) variable region. In an embodiment, the variable region comprises rodent e.g., murine or lagomorph, e.g., rabbit CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the term “cancer” refers to any condition characterized by the uncontrolled division of abnormal cells in the body. For example, mutations can occur in a cell that prevent it from being able to regulate cell division and result in the formation of one or more tumors. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

A “cell” or “cells,” as used herein, refers to the basic structural and functional unit of a living organism. A cell comprises a membrane-bound cytoplasm containing biological macromolecules (e.g., nucleic acids, carbohydrates, lipids, and proteins) and organelles necessary to sustain life. A “cancer cell,” as used herein, refers to an abnormal cell (e.g., one that has accumulated one or more deleterious mutations) undergoing uncontrolled cell division. A healthy cell may be derived from healthy tissue, such as skin tissue, while a cancer cell may be derived from a pathological tissue, such as a tumor.

As used herein, the term “interaction” refers to the non-covalent chemical bonds that form between biological macromolecules for a functionally relevant duration of time. A “collagen interaction,” as used herein in reference to DDR1, refers to non-covalent chemical bonds that occur between the extracellular domains of the DDR1 protein and collagen with a strength and duration that is sufficient to promote autophosphorylation of the intracellular domains of DDR1.

As used interchangeably herein, the terms “DDR1 related disease,” “DDR1 related disorder” or “DDR1 related condition” refers to any pathological state associated with or directly caused by aberrant expression and/or function of DDR1. For example, the DDR1 related disorder may comprise a cancer in which overexpression of DDR1 suppresses antitumor immunity, thereby preventing recognition and clearance of the tumor. By way of further example, the DDR1 related disorder may comprise a fibrotic condition in which overexpression of DDR1 is associated with excess accumulation of extracellular matrix components (e.g., collagen) and impaired tissue functionality.

The terms “effective,” “effective amount,” “therapeutically effective amount,” and “pharmaceutically effective amount,” as used interchangeably herein in reference to a treatment, refer an amount of an agent that is sufficient to achieve a desired biological result. That result may be a reduction and/or alleviation in the severity, duration and/or frequency of one or more sign, symptom, side-effect and/or cause of a disease or disorder being treated.

As used herein, the terms “fibrosis” and “fibrotic conditions” refer to any condition that is characterized by the replacement of normal parenchymal tissue with connective tissue. For example, damage or inflammation of a tissue can result in the excess accumulation of extracellular matrix components (e.g., collagen). When severe enough, this accumulation can interfere with the normal architecture and/or function of the tissue.

As used herein, a “reference sample” refers to one or more biological samples or derivatives thereof comprising DDR1, which can be compared to a subject sample. A “positive reference sample,” as used herein, refers to a sample in which an entity of interest is known to be present and/or a condition is known to be met for the purposes of comparison. For example, a positive reference sample may comprise a sample derived from cancerous tissue known to overexpress phosphorylated DDR1. By contrast, a “negative reference sample,” as used herein, refers to a sample in which an entity of interest is known to be absent and/or a condition is known not to be met for the purposes of comparison. For example, a negative reference sample may comprise a sample derived from healthy tissue, such as skin tissue, known to express normal levels of phosphorylated DDR1. A “reference cell,” as used herein, refers to one or more cells comprising phosphorylated DDR1, which can be compared to a subject cell. A “positive reference cell,” as used herein, refers to a cell in which an entity of interest is known to be present and/or a condition is known to be met for the purposes of comparison. For example, a positive reference cell may comprise a cell derived from cancerous tissue known to overexpress phosphorylated DDR1. By contrast, a “negative reference cell,” as used herein, refers to a cell in which an entity of interest is known to be absent and/or a condition is known not to be met for the purposes of comparison. For example, a negative reference sample may comprise a cell derived from healthy tissue, such as skin tissue, known to express normal levels of phosphorylated DDR1.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “tissue” refers to a grouping of interconnected cells that share a common biological origin within an organism. By way of example, the tissue may perform a physiological function in vivo (e.g., lung tissue for allowing gas transfer) or be the result of a pathological state (e.g., tumor tissue as a product of cancer, fibrotic tissue as a product of excess inflammation, etc.).

As used herein, the term “EU numbering system” refers to the EU numbering convention for the constant regions of an antibody, as described in Edelman G M et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody or polynucleotide, the term “isolated” refers to an antibody or polynucleotide that is separated from one or more contaminants (e.g., polypeptides, polynucleotides, lipids, or carbohydrates, etc.) which are present in a natural source of the antibody or polynucleotide. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

The determination of “percent identity” between two sequences (e.g., amino acid sequences or nucleic acid sequences) can be accomplished using a mathematical algorithm. A non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 7.2 Anti-DDR1 Antibodies

In one aspect, the instant disclosure provides methods of screening for, determining the effectiveness of, or determining the likely effectiveness of one or more inhibitor of DDR1. In some embodiments, the inhibitor of DDR1 comprises an antibody specific to DDR1 (i.e., anti-DDR1 antibodies).

In one embodiment, the instant disclosure provides an isolated antibody that specifically binds to DDR1 (anti-DDR1). The amino acid sequences of the CDR and VH/VL of exemplary antibodies that specifically bind to DDR1 are set forth in Tables 2 and 3, respectively.

TABLE 2
amino acid sequences of exemplary
anti-DDR1 antibodies.

| Antibody | | | | | | |
|---|---|---|---|---|---|---|
| | | SEQ | | SEQ | | SEQ |
| | | ID | | ID | | ID |
| | CDRL1 | NO: | CDRL2 | NO: | CDRL3 | NO: |
| rabbit | ETISSR | 5 | QAS | | QGCYYGG | 7 |
| DDR1-33 | | | | | GSFYDSA | |
| chimeric | ETISSR | 5 | QAS | | QGCYYGG | 7 |
| DDR1-33 | | | | | GSFYDSA | |
| humanized | QSIGSV | 17 | GVF | | QYIPYGS | 19 |
| DDR1-9 | | | | | SP | |
| | | SEQ | | SEQ | | SEQ |
| | | ID | | ID | | ID |
| | CDRH1 | NO: | CDRH2 | NO: | CDRH3 | NO: |
| rabbit | GFSLSSYD | 8 | SWNS | 9 | ARLGADD | 10 |
| DDR1-33 | | | GFV | | IYYFNL | |
| chimeric | GFSLSSYD | 8 | SWNS | 9 | ARLGADD | 10 |
| DDR1-33 | | | GFV | | IYYFNL | |
| humanized | GFSLNRYY | 20 | ISYG | 21 | ARADTGD | 22 |
| DDR1-9 | | | DTT | | NGYLGLQ | |
| | | | | | L | |

TABLE 3
VH/VL amino acid sequences of exemplary anti-DDR1 antibodies.

| | | |
|---|---|---|
| | SEQ ID | |
| Antibody | NO: | Sequence |

Light chain variable regions

| | | |
|---|---|---|
| rabbit DDR1-33 | 3 | ELVMTQTPASVEAAVGGTVTIKCQASETISSRLAWYQQKPGQPPKLLIYQASK |
| | | LPSGVPSRFKGTGSGTEYTLTISDLECADAATYYCQGCYYGGGSFYDSAFGGG |
| | | TEVVVK |
| chimeric DDR1-33 | 3 | ELVMTQTPASVEAAVGGTVTIKCQASETISSRLAWYQQKPGQPPKLLIYQASK |
| | | LPSGVPSRFKGTGSGTEYTLTISDLECADAATYYCQGCYYGGGSFYDSAFGGG |
| | | TEVVVK |
| humanized DDR1-9_1 | 11 | DIQMTQSPSSVSASVGDRVTITCQASQSIGSVLAWYQQKPGKAPKLLISGVED |
| | | LASGVPSRFSGSGSGTDFTLTISSLQPEDFATYYCQYIPYGSSPFGGGTKVEI |
| | | K |
| humanized DDR1-9 2 | 12 | DIQMTQSPSSVSASVGDRVTITCRASQSIGSVLAWYQQKPGKAPKLLIYGVFS |
| | | LQSGVPSRFSGSGSGTDFTLTISSLQPEDFATYYCQYIPYGSSPFGGGTKVEI |
| | | K |

Heavy chain variable regions

| | | |
|---|---|---|
| rabbit DDR1-33 | 4 | QSVEESGGRLVTPGGSLTLTCTVSGFSLSSYDMSWVRQAPGKGLEWIGISWNS |
| | | GFVDYASWAKGRESISKTSTTVDLKITSPTTEDTATYFCARLGADDIYYFNLW |
| | | GPGTLVTISS |
| chimeric DDR1-33 | 4 | QSVEESGGRLVTPGGSLTLTCTVSGFSLSSYDMSWVRQAPGKGLEWIGISWNS |
| | | GFVDYASWAKGRESISKTSTTVDLKITSPTTEDTATYFCARLGADDIYYFNLW |
| | | GPGTLVTISS |
| humanized DDR1-9 | 13 | QVQLVESGGRVVQPGRSLRLSCTASGFSLNRYYMLWVRQAPGKGLEWIGTISY |
| | | GDTTYYASWAKGRFTISRDNSKNTLYLQMNSLRAEDTAVYYCARADTGDNGYL |
| | | GLQLWGQGTLVTVSS |

In various embodiments, the instant disclosure provides an isolated antibody that specifically binds to DDR1 (anti-DDR1), the anti-DDR1 antibody comprising a CDRL1, CDRL2 and CDRL3 set forth in Table 2. In one embodiment, the anti-DDR1 antibody comprises a CDRL1 as set forth in Table 2. In one embodiment, the anti-DDR1 antibody comprises a CDRL2 as set forth in Table 2. In one embodiment, the anti-DDR1 antibody comprises a CDRL3 as set forth in Table 2.

In one embodiment, the anti-DDR1 antibody comprises a VL domain comprising one, two or all three of the CDRs of a VL domain disclosed in Table 3. In one embodiment, the anti-DDR1 antibody comprises the CDRL1 of a VL domain set forth in Table 3. In one embodiment, the anti-DDR1 antibody comprises the CDRL2 of a VL domain set forth in Table 3. In one embodiment, the anti-DDR1 antibody comprises the CDRL3 of a VL domain set forth in Table 3. In one embodiment, the anti-DDR1 antibody comprises a VL domain as set forth in Table 3.

In various embodiments, the instant disclosure provides an isolated antibody that specifically binds to DDR1 (anti-DDR1), the anti-DDR1 antibody comprising a CDRH1, CDRH2 and CDRH3 set forth in Table 2. In one embodiment, the anti-DDR1 antibody comprises a CDRH1 as set forth in Table 2. In one embodiment, the anti-DDR1 antibody comprises a CDRH2 as set forth in Table 2. In one embodiment, the anti-DDR1 antibody comprises a CDRH3 as set forth in Table 2.

In one embodiment, the anti-DDR1 antibody comprises a VH domain comprising one, two or all three of the CDRs of a VH domain disclosed in Table 3. In one embodiment, the anti-DDR1 antibody comprises the CDRH1 of a VH domain set forth in Table 3. In one embodiment, the anti-DDR1 antibody comprises the CDRH2 of a VH domain set forth in Table 3. In one embodiment, the anti-DDR1 antibody comprises the CDRH3 of a VH domain set forth in Table 3. In one embodiment, the anti-DDR1 antibody comprises a VH domain as set forth in Table 3.

In one embodiment, the anti-DDR1 antibody comprises the CDRL1 of SEQ ID NO: 5 or 17. In one embodiment, the anti-DDR1 antibody comprises the CDRL2 of QAS or GVF. In one embodiment, the anti-DDR1 antibody comprises the CDRL3 of SEQ ID NO: 7 or 19. In one embodiment, the anti-DDR1 antibody comprises at least two of the CDRL1, CDRL2 or CDRL3 as set forth in SEQ ID NOs: 5-7 or 17-19. In one embodiment, the anti-DDR1 antibody comprises the CDRL1, CDRL2 and CDRL3 of SEQ ID NOs: 5-7 or SEQ ID NOs: 17-19.

In one embodiment, the anti-DDR1 antibody comprises the CDRL1 of SEQ ID NO: 5. In one embodiment, the anti-DDR1 antibody comprises the CDRL2 of QAS. In one embodiment, the anti-DDR1 antibody comprises the CDRL3 of SEQ ID NO: 7. In one embodiment, the anti-DDR1 antibody comprises at least two of the CDRL1, CDRL2 or CDRL3 as set forth in SEQ ID NOs: 5-7. In one embodiment, the anti-DDR1 antibody comprises the CDRL1, CDRL2 and CDRL3 of SEQ ID NOs: 5-7.

In one embodiment, the anti-DDR1 antibody comprises the CDRL1 of SEQ ID NO: 17. In one embodiment, the anti-DDR1 antibody comprises the CDRL2 of GVF. In one embodiment, the anti-DDR1 antibody comprises the CDRL3 of SEQ ID NO: 19. In one embodiment, the anti-DDR1 antibody comprises at least two of the CDRL1, CDRL2 or CDRL3 as set forth in SEQ ID NOs: 17-19. In one embodiment, the anti-DDR1 antibody comprises the CDRL1, CDRL2 and CDRL3 of SEQ ID NOs: 17-19.

In one embodiment, the anti-DDR1 antibody comprises a VL domain comprising one, two or all three of the CDRs of the VL domain of SEQ ID NO: 3. In one embodiment, the anti-DDR1 antibody comprises the VL domain of SEQ ID NO: 3. In one embodiment, the anti-DDR1 antibody comprises a VL domain comprising one, two or all three of the CDRs of the VL domain of SEQ ID NO: 11. In one embodiment, the anti-DDR1 antibody comprises the VL domain of SEQ ID NO: 11. In one embodiment, the anti-DDR1 antibody comprises a VL domain comprising one, two or all three of the CDRs of the VL domain of SEQ ID NO: 12. In one embodiment, the anti-DDR1 antibody comprises the VL domain of SEQ ID NO: 12.

In one embodiment, the anti-DDR1 antibody comprises a light chain (LC) comprising the VL of a LC sequence as set forth in Table 4 below. In one embodiment, the anti-DDR1 antibody comprises a LC as set forth in Table 4.

TABLE 4
LC/HC amino acid sequences of exemplary anti-DDR1 antibodies.

| | | |
|---|---|---|
| | SEQ | |
| | ID | |
| Antibody | NO: | Sequence |

Light Chain

| | | |
|---|---|---|
| rabbit | 164 | ELVMTQTPASVEAAVGGTVTIKCQASETISSRLAWYQQKPGQPPKLLIYQASKLPSGVPSRFK |
| DDR1-33 | | GTGSGTEYTLTISDLECADAATYYCQGCYYGGGSFYDSAFGGGTEVVVKGDPVAPTVLIFPPA |
| | | ADQVATGTVTIVCVANKYFPDVTVTWEVDGTTQTTGIENSKTPQNSADCTYNLSSTLTLTSTQ |
| | | YNSHKEYTCKVTQGTTSVVQSFNRGDC |
| chimeric | 23 | ELVMTQTPASVEAAVGGTVTIKCQASETISSRLAWYQQKPGQPPKLLIYQASKLPSGVPSRFK |
| DDR1-33 | | GTGSGTEYTLTISDLECADAATYYCQGCYYGGGSFYDSAFGGGTEVVVKRTVAAPSVFIFPPS |
| | | DEQLKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| humanized | 161 | DIQMTQSPSSVSASVGDRVTITCQASQSIGSVLAWYQQKPGKAPKLLISGVFDLASGVPSRFS |
| DDR1-9 1 | | GSGSGTDFTLTISSLQPEDFATYYCQYIPYGSSPFGGGTKVEIKRTVAAPSVFIFPPSDEQLK |
| | | SGTASVVCLLNNFYPREAKVQWKVDNALQSGNSQESVTEQDSKDSTYSLSSTLTLSKADYEKH |
| | | KVYACEVTHQGLSSPVTKSENRGEC |

Heavy Chain

| | | |
|---|---|---|
| rabbit | 165 | QSVEESGGRLVTPGGSLTLTCTVSGFSLSSYDMSWVRQAPGKGLEWIGISWNSGFVDYASWAK |
| DDR1-33 | | GRESISKTSTTVDLKITSPTTEDTATYFCARLGADDIYYFNLWGPGTLVTISSGQPKAPSVFP |
| | | LAPCCGDTPSSTVTLGCLVKGYLPEPVTVTWNSGTLINGVRTFPSVRQSSGLYSLSSVVSVTS |
| | | SSQPVTCNVAHPATNTKVDKTVAPSTCSKPTCPPPELLGGPSVFIFPPKPKDTLMISRTPEVT |
| | | CVVVDVSQDDPEVQFTWYINNEQVRTARPPLREQQFNSTIRVVSTLPIAHQDWLRGKEFKCKV |
| | | HNKALPAPIEKTISKARGQPLEPKVYTMGPPREELSSRSVSLTCMINGFYPSDISVEWEKNGK |
| | | AEDNYKTTPAVLDSDGSYFLYSKLSVPTSEWQRGDVFTCSVMHEALHNHYTQKSISRSPGK |
| chimeric | 24 | QSVEESGGRLVTPGGSLTLTCTVSGFSLSSYDMSWVRQAPGKGLEWIGISWNSGFVDYASWAK |
| DDR1-33 | | GRFSISKTSTTVDLKITSPTTEDTATYFCARLGADDIYYFNLWGPGTLVTISSASTKGPSVFP |
| | | LAPSSKSTSGGTAALGCLVKDYFPEPVTVSWNSGALTSGVHTFPAVLQSSGLYSLSSVVTVPS |
| | | SSLGTQTYICNVNHKPSNTKVDKRVEPKSCDKTHTCPPCPAPEAEGAPSVFLFPPKPKDTLMI |
| | | SRTPEVTCVVVDVSHEDPEVKFNWYVDGVEVHNAKTKPREEQYNSTYRVVSVLTVLHQDWLNG |
| | | KEYKCKVSNKALPSSIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEALHNHYTQKSLS |
| | | LSPGK |
| humanized | 162 | QVQLVESGGRVVQPGRSLRLSCTASGFSLNRYYMLWVRQAPGKGLEWIGTISYGDTTYYASWA |
| DDR1-9 1 | | KGRFTISRDNSKNTLYLQMNSLRAEDTAVYYCARADTGDNGYLGLQLWGQGTLVTVSSASTKG |
| inert | | PSVFPLAPSSKSTSGGTAALGCLVKDYFPEPVTVSWNSGALTSGVHTFPAVLQSSGLYSLSSV |
| IgG1 Fc | | VTVPSSSLGTQTYICNVNHKPSNTKVDKRVEPKSCDKTHTCPPCPAPEAEGAPSVFLFPPKPK |
| | | DTLMISRTPEVTCVVVDVSHEDPEVKFNWYVDGVEVHNAKTKPREEQYNSTYRVVSVLTVLHQ |
| | | DWLNGKEYKCKVSNKALPSSIEKTISKAKGQPREPQVYTLPPSREEMTKNQVSLTCLVKGFYP |
| | | SDIAVEWESNGQPENNYKTTPPVLDSDGSFFLYSKLTVDKSRWQQGNVFSCSVMHEALHNHYT |
| | | QKSLSLSPGK |
| humanized | 163 | QVQLVESGGRVVQPGRSLRLSCTASGFSLNRYYMLWVRQAPGKGLEWIGTISYGDTTYYASWA |
| DDR1-9_1 | | KGRFTISRDNSKNTLYLQMNSLRAEDTAVYYCARADTGDNGYLGLQLWGQGTLVTVSSASTKG |
| WT IgG1 Fc | | PSVFPLAPSSKSTSGGTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPELLGGPSVFLFPPKPK |
| | | DTLMISRTPEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQVSLTCLVKGFYP |
| | | SDIAVEWESNGQPENNYKTTPPVLDSDGSFFLYSKLTVDKSRWQQGNVFSCSVMHEALHNHYT |
| | | QKSLSLSPGK |

In one embodiment, the anti-DDR1 antibody comprises a light chain comprising the VL of the light chain of SEQ ID NO: 23. In one embodiment, the anti-DDR1 antibody comprises the light chain of SEQ ID NO: 23.

In one embodiment, the anti-DDR1 antibody comprises a light chain comprising the VL of the light chain of SEQ ID NO: 161. In one embodiment, the anti-DDR1 antibody comprises the light chain of SEQ ID NO: 161.

In one embodiment, the anti-DDR1 antibody comprises a light chain comprising the VL of the light chain of SEQ ID NO: 164. In one embodiment, the anti-DDR1 antibody comprises the light chain of SEQ ID NO: 164.

In one embodiment, the anti-DDR1 antibody comprises the CDRH1 of SEQ ID NO: 8 or 20. In one embodiment, the anti-DDR1 antibody comprises the CDRH2 of SEQ ID NO: 9 or 21. In one embodiment, the anti-DDR1 antibody comprises the CDRH3 of SEQ ID NO: 10 or 22. In one embodiment, the anti-DDR1 antibody comprises at least two of the CDRH1, CDRH2 or CDRH3 as set forth in SEQ ID NOs: 8-10 or 20-22. In one embodiment, the anti-DDR1 antibody comprises the CDRH1, CDRH2 and CDRH3 of SEQ ID NOs: 8-10 or SEQ ID NOs: 20-22.

In one embodiment, the anti-DDR1 antibody comprises the CDRH1 of SEQ ID NO: 8. In one embodiment, the anti-DDR1 antibody comprises the CDRH2 of SEQ ID NO: 9. In one embodiment, the anti-DDR1 antibody comprises the CDRH3 of SEQ ID NO: 10. In one embodiment, the anti-DDR1 antibody comprises at least two of the CDRH1, CDRH2 or CDRH3 as set forth in SEQ ID NOs: 8-10. In one embodiment, the anti-DDR1 antibody comprises the CDRH1, CDRH2 and CDRH3 of SEQ ID NOs: 8-10.

In one embodiment, the anti-DDR1 antibody comprises the CDRH1 of SEQ ID NO: 20. In one embodiment, the anti-DDR1 antibody comprises the CDRH2 of SEQ ID NO: 21. In one embodiment, the anti-DDR1 antibody comprises the CDRH3 of SEQ ID NO: 22. In one embodiment, the anti-DDR1 antibody comprises at least two of the CDRH1, CDRH2 or CDRH3 as set forth in SEQ ID NOs: 20-22. In one embodiment, the anti-DDR1 antibody comprises the CDRH1, CDRH2 and CDRH3 of SEQ ID NOs: 20-22.

In one embodiment, the anti-DDR1 antibody comprises a VH domain comprising one, two or all three of the CDRs of the VH domain of SEQ ID NO: 4. In one embodiment, the anti-DDR1 antibody comprises the VH domain of SEQ ID NO: 4. In one embodiment, the anti-DDR1 antibody comprises a VH domain comprising one, two or all three of the CDRs of the VH domain of SEQ ID NO: 13. In one embodiment, the anti-DDR1 antibody comprises the VH domain of SEQ ID NO: 13.

In one embodiment, the anti-DDR1 antibody comprises a heavy chain comprising the VH of the heavy chain of SEQ ID NO: 24. In one embodiment, the anti-DDR1 antibody comprises the heavy chain of SEQ ID NO: 24.

In one embodiment, the anti-DDR1 antibody comprises a heavy chain comprising the VH of the heavy chain of SEQ ID NO: 162. In one embodiment, the anti-DDR1 antibody comprises the heavy chain of SEQ ID NO: 162.

In one embodiment, the anti-DDR1 antibody comprises a heavy chain comprising the VH of the heavy chain of SEQ ID NO: 163. In one embodiment, the anti-DDR1 antibody comprises the heavy chain of SEQ ID NO: 163.

In one embodiment, the anti-DDR1 antibody comprises a heavy chain comprising the VH of the heavy chain of SEQ ID NO: 165. In one embodiment, the anti-DDR1 antibody comprises the heavy chain of SEQ ID NO: 165.

In one embodiment, the anti-DDR1 antibody comprises the CDRL1, CDRL2 and CDRL3 of SEQ ID NO: 5, QAS, and SEQ ID NO: 7, respectively; and the CDRH1, CDRH2 and CDRH3 of SEQ ID NO: 8, SEQ ID NO: 9 and SEQ ID NO: 10, respectively. In one embodiment, the anti-DDR1 antibody comprises the VL domain of SEQ ID NO: 3; and the VH domain of SEQ ID NO: 4.

In one embodiment, the anti-DDR1 antibody comprises the CDRL1, CDRL2 and CDRL3 of SEQ ID NO: 17, GVF and SEQ ID NO: 19, respectively; and the CDRH1, CDRH2 and CDRH3 of SEQ ID NO: 20, SEQ ID NO: 21 and SEQ ID NO: 22, respectively. In one embodiment, the anti-DDR1 antibody comprises the VL domain of SEQ ID NO: 11; and the VH domain of SEQ ID NO: 13. In one embodiment, the anti-DDR1 antibody comprises the VL domain of SEQ ID NO: 12; and the VH domain of SEQ ID NO: 13.

In one embodiment, the anti-DDR1 antibody comprises the light chain of SEQ ID NO: 23; and the heavy chain of SEQ ID NO: 24. In one embodiment, the anti-DDR1 antibody comprises the light chain of SEQ ID NO: 161; and the heavy chain of SEQ ID NO: 162. In one embodiment, the anti-DDR1 antibody comprises the light chain of SEQ ID NO: 161; and the heavy chain of SEQ ID NO: 163. In one embodiment, the anti-DDR1 antibody comprises the light chain of SEQ ID NO: 164; and the heavy chain of SEQ ID NO: 165.

In some embodiments, the anti-DDR1 antibody or antigen-binding fragment thereof comprises a light chain variable region having a CDRL1, CDRL2 and CDRL3 selected from the CDRL1, CDRL2 and CDRL3 sequences for each mAb set forth in Table 5 below; and a heavy chain variable region having CDRH1, CDRH2 and CDRH3 selected from the CDRH1, CDRH2 and CDRH3 sequences for each mAb set forth in Table 6 below, or variants thereof wherein one or more CDRLs and/or CDRHs has one, two or three amino acid substitutions, additions deletions, or combinations thereof.

TABLE 5
CDRs of light chain amino acid variable region sequences of DDR1 antibodies.

| mAb Name | CDR1 | SEQ ID NO: | CDR2 | CDR3 | SEQ ID NO: |
|---|---|---|---|---|---|
| DDR1-1 | QNIYSN | 25 | GAS | QSGYYSSSTDIA | 44 |
| DDR1-3 | QTISSW | 26 | YAF | QQGISSSNVDNV | 45 |
| DDR1-5 | QTISSW | 27 | YAF | QCTYGSGSSSSYGCA | 46 |
| DDR1-6 | QSVYSNY | 28 | ETS | QGGYSEIIENT | 47 |
| DDR1-11 | QSIGSTY | 29 | KAS | LYGGFGSSTGDA | 48 |
| DDR1-12 | QTIYSN | 30 | QAS | QSYYGADDYT | 49 |
| DDR1-13 | KSVYNNNA | 31 | GVS | AGDYSDISDNN | 50 |
| DDR1-14 | QSISSY | 32 | EAS | QNNNGFSGSNENN | 51 |
| DDR1-15 | QTIYSS | 33 | KAS | QQGSSISNVDKNA | 52 |
| DDR1-17 | QSIGSY | 34 | EAS | QNNNGMTVSDENA | 53 |
| DDR1-20 | QIIDHDH | 35 | RAS | QNNNGMTVSDENA | 54 |
| DDR1-21 | QSVVDKNW | 36 | EAS | AGDFESGVSG | 55 |
| DDR1-22 | KNIYNNNA | 37 | GAS | AADYSDISDNN | 56 |
| DDR1-23 | QSVYSNNY | 38 | AAS | LGGYNDDAN | 57 |
| DDR1-26 | ESVYSNNH | 39 | AAS | LGGYNDDAN | 58 |
| DDR1-28 | QSIDNND | 40 | RTS | QSYCVNTYGYT | 59 |
| DDR1-29 | QSISNH | 41 | RAS | QSYYIINRSNYANS | 60 |
| DDR1-32 | ESINSW | 42 | DAS | QSYYIINRSNYGNS | 61 |
| DDR1-34 | ENLYKDNY | 43 | GAS | AGGYDSVVD | 62 |

TABLE 6
CDRs of heavy chain amino acid variable region sequences of DDR1 antibodies.

| | | SEQ | | SEQ | | SEQ |
|---|---|---|---|---|---|---|
| | | ID | | ID | | ID |
| mAb Name | CDR1 | NO: | CDR2 | NO: | CDR3 | NO: |
| DDR1-1 | GFSLSRYA | 63 | IGSSGLT | 82 | ARGMWYDDSDDYEDYFNL | 101 |
| DDR1-3 | GIDLSSYA | 64 | INIGGGT | 83 | ARDVDAHTLTYFTL | 102 |
| DDR1-5 | GFTLSNNA | 65 | IYASGRT | 84 | ARGDTETDYGIPYFDL | 103 |
| DDR1-6 | GFSFSSSYY | 66 | IYASSGST | 85 | AILGADYRLTRLDL | 104 |
| DDR1-11 | GFSFSSGYY | 67 | IYTGRTDFT | 86 | ARGDYSGGVGGNYWLDL | 105 |
| DDR1-12 | GIDLSNTW | 68 | ITDSGTT | 87 | GRDPGDITSGTNDL | 106 |
| DDR1-13 | SGFSLNNY | 69 | IFNNGDI | 88 | ARTGYRTGGWL | 107 |
| DDR1-14 | GIDLSYYA | 70 | INGRGDT | 89 | AREDSAIPFIVGNYYGMDL | 108 |
| DDR1-15 | TFSFNSRYW | 71 | INNGDIS | 90 | AKGGNLAGDCYGL | 109 |
| DDR1-17 | GFSLNRYA | 72 | IGSSGST | 91 | ARDLDDSYGYTYATGMDIRLDL | 110 |
| DDR1-20 | GFSLSDYA | 73 | INSRDDT | 92 | AREDSSIPFIVGNYYGMDL | 111 |
| DDR1-21 | GFSLSSYG | 74 | IYPSGSI | 93 | VRYLTGSSDLHL | 112 |
| DDR1-22 | GFSLSDYA | 75 | INNGDIY | 94 | ARPGYRTGIWL | 113 |
| DDR1-23 | GFDLRSYYY | 76 | IHGGEGNT | 95 | RGGWTNYF | 114 |
| DDR1-26 | GFDLSSNYY | 77 | IYSSNTRT | 96 | RGGWTNYL | 115 |
| DDR1-28 | GFSLSSHD | 78 | IISSGNT | 97 | ARDVYSGASP | 116 |
| DDR1-29 | TFSFNSRYW | 79 | INNGDIT | 98 | AKGGNLAGDCYGL | 117 |
| DDR1-32 | GFSLSSYY | 80 | ITTAGPL | 99 | ARGHAGSIYYSYFDL | 118 |
| DDR1-34 | GFDLSSYYY | 81 | IYTSSGAT | 100 | RGGWCDFNL | 119 |

In some embodiments, the anti-DDR1 antibody or antigen-binding fragment thereof comprises a light chain variable region having a CDRL1, CDRL2 and CDRL3 comprising the amino acid sequences of QNIYSN (SEQ ID NO: 25), GAS and QSGYYSSSTDIA (SEQ ID NO: 44), respectively; and a heavy chain variable region having a CDRH1, CDRH2 and CDRH3 comprising the amino acid sequences of GFSLSRYA (SEQ ID NO: 63), IGSSGLT (SEQ ID NO: 82), and ARGMWYDDSDDYEDYFNL (SEQ ID NO: 101), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QTISSW (SEQ ID NO: 26), YAF and QQGISSSNVDNV (SEQ ID NO: 45), respectively; and a heavy chain variable region having a CDRH1, CDRH2 and CDRH3 comprising the amino acid sequences of GIDLSSYA (SEQ ID NO: 64), INIGGGT (SEQ ID NO: 83), and ARDVDAHTLTYFTL (SEQ ID NO: 102), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QTISSW (SEQ ID NO: 27), YAF and QCTYGSGSSSSYGCA (SEQ ID NO: 46), respectively; and a heavy chain variable region having a CDRH1, CDRH2 and CDRH3 comprising the amino acid sequences of GFTLSNNA (SEQ ID NO: 65), IYASGRT (SEQ ID NO: 84), and ARGDTETDYGIPYFDL (SEQ ID NO: 103), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSVYSNY (SEQ ID NO: 28), ETS and QGGYSEIIENT (SEQ ID NO: 47), respectively; and a heavy chain variable region having a CDRH1, CDRH2 and CDRH3 comprising the amino acid sequences of GFSFSSSYY (SEQ ID NO: 66), IYASSGST (SEQ ID NO: 85), and AILGADYRLTRLDL (SEQ ID NO: 104), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSIGSTY (SEQ ID NO: 29), KAS and LYGGFGSSTGDA (SEQ ID NO: 48), respectively; and a heavy chain variable region having a CDRH1, CDRH2 and CDRH3 comprising the amino acid sequences of GFSFSSGYY (SEQ ID NO: 67), IYTGRTDFT (SEQ ID NO: 86), and ARGDYSGGVGGNYWLDL (SEQ ID NO: 105), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QTIYSN (SEQ ID NO: 30), QAS and QSYYGADDYT (SEQ ID NO: 49), respectively; and a heavy chain variable region having a CDRH1, CDRH2 and CDRH3 comprising the amino acid sequences of GIDLSNTW (SEQ ID NO: 68), ITDSGTT (SEQ ID NO: 87), and GRDPGDITSGTNDL (SEQ ID NO: 106), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of KSVYNNNA (SEQ ID NO: 31), GVS and AGDYSDISDNN (SEQ ID NO: 50), respectively; and a heavy chain variable region having a CDRH1, CDRH2 and CDRH3 comprising the amino acid sequences of SGFSLNNY (SEQ ID NO: 69), IFNNGDI (SEQ ID NO: 88), and ARTGYRTGGWL (SEQ ID NO: 107), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSISSY (SEQ ID NO: 32), EAS and QNNNGFSGSNENN (SEQ ID NO: 51), respectively; and a heavy chain variable region having a CDRH1, CDRH2 and CDRH3 comprising the amino acid sequences of GIDLSYYA (SEQ ID NO: 70), INGRGDT (SEQ ID NO: 89), and AREDSAIPFIVGNYYGMDL (SEQ ID NO: 108), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QTIYSS (SEQ ID NO: 33), KAS and QQGSSISNVDKNA (SEQ ID NO: 52), respectively; and a heavy chain variable region having a CDRH1, CDRH2 and CDRH3 comprising the amino acid sequences of TFSFNSRYW (SEQ ID NO: 71), INNGDIS (SEQ ID NO: 90), and AKGGNLAGDCYGL (SEQ ID NO: 109), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSIGSY (SEQ ID NO: 34), EAS and QNNNGMTVSDFNA (SEQ ID NO: 53), respectively; and a heavy chain variable region having a CDRH1, CDRH2 and CDRH3 comprising the amino acid sequences of GFSLNRYA (SEQ ID NO: 72), IGSSGST (SEQ ID NO: 91), and ARDLDDSYGYTYATGMDIRLDL (SEQ ID NO: 110), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QIIDHDH (SEQ ID NO: 35), RAS and QNNNGMTVSDFNA (SEQ ID NO: 54), respectively; and a heavy chain variable region having a CDRH1, CDRH2 and CDRH3 comprising the amino acid sequences of GFSLSDYA (SEQ ID NO: 73), INSRDDT (SEQ ID NO: 92), and AREDSSIPFIVGNYYGMDL (SEQ ID NO: 111), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSVVDKNW (SEQ ID NO: 36), EAS and AGDFESGVSG (SEQ ID NO: 55), respectively; and a heavy chain variable region having a CDRH1, CDRH2 and CDRH3 comprising the amino acid sequences of GFSLSSYG (SEQ ID NO: 74), IYPSGSI (SEQ ID NO: 93), and VRYLTGSSDLHL (SEQ ID NO: 112), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of KNIYNNNA (SEQ ID NO: 37), GAS and AADYSDISDNN (SEQ ID NO: 56), respectively; and a heavy chain variable region having a CDRH1, CDRH2 and CDRH3 comprising the amino acid sequences of GFSLSDYA (SEQ ID NO: 75), INNGDIY (SEQ ID NO: 94), and ARPGYRTGIWL (SEQ ID NO: 113), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSVYSNNY (SEQ ID NO: 38), AAS and LGGYNDDAN (SEQ ID NO: 57), respectively; and a heavy chain variable region having a CDRH1, CDRH2 and CDRH3 comprising the amino acid sequences of GFDLRSYYY (SEQ ID NO: 76), IHGGEGNT (SEQ ID NO: 95), and RGGWTNYF (SEQ ID NO: 114), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of ESVYSNNH (SEQ ID NO: 39), AAS and LGGYNDDAN (SEQ ID NO: 58), respectively; and a heavy chain variable region having a CDRH1, CDRH2 and CDRH3 comprising the amino acid sequences of GFDLSSNYY (SEQ ID NO: 77), IYSSNTRT (SEQ ID NO: 96), and RGGWTNYL (SEQ ID NO: 115), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSIDNND (SEQ ID NO: 40), RTS and QSYCVNTYGYT (SEQ ID NO: 59), respectively; and a heavy chain variable region having a CDRH1, CDRH2 and CDRH3 comprising the amino acid sequences of GFSLSSHD (SEQ ID NO: 78), IISSGNT (SEQ ID NO: 97), and ARDVYSGASP (SEQ ID NO: 116), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of QSISNH (SEQ ID NO: 41), RAS and QSYYIINRSNYANS (SEQ ID NO: 60), respectively; and a heavy chain variable region having a CDRH1, CDRH2 and CDRH3 comprising the amino acid sequences of TFSFNSRYW (SEQ ID NO: 79), INNGDIT (SEQ ID NO: 98), and AKGGNLAGDCYGL (SEQ ID NO: 117), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of ESINSW (SEQ ID NO: 42), DAS and QSYYIINRSNYGNS (SEQ ID NO: 61), respectively; and a heavy chain variable region having a CDRH1, CDRH2 and CDRH3 comprising the amino acid sequences of GFSLSSYY (SEQ ID NO: 80), ITTAGPL (SEQ ID NO: 99), and ARGHAGSIYYSYFDL (SEQ ID NO: 118), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a CDRL1, CDRL2 and CDRL3 comprising the amino acid sequences of ENLYKDNY (SEQ ID NO: 43), GAS and AGGYDSVVD (SEQ ID NO: 62), respectively; and a heavy chain variable region having a CDRH1, CDRH2 and CDRH3 comprising the amino acid sequences of GFDLSSYYY (SEQ ID NO: 81), IYTSSGAT (SEQ ID NO: 100), and RGGWCDFNL (SEQ ID NO: 119), respectively, or variants thereof wherein one or more CDRLs and/or CDRHs has one, two or three amino acid substitutions, additions deletions, or combinations thereof.

In some embodiments, the anti-DDR1 antibody or antigen-binding fragment thereof comprises a light chain variable region having a light chain variable region amino acid sequence selected from the sequences presented in Table 7 below (e.g., SEQ ID NOs: 120-139). In some embodiments, the anti-DDR1 antibody or antigen-binding fragment thereof comprises a heavy chain variable region having a heavy chain variable region amino acid sequence selected from the sequences presented in Table 8 below (e.g., SEQ ID NOs: 140-159).

TABLE 7
Light chain variable region amino acid sequences of anti-DDR1 antibodies.

| mAb Name | Light chain variable amino acid sequences | SEQ ID NO: |
|---|---|---|
| DDR1-1K | ELVLTQTPASVSAAVGGTVTINCQASQNIYSNLAWYQQKPGQPPKLLIY | 120 |
| | GASNLESGVPSRFKGSGSGTQFTLTISDLECDDAATYYCQSGYYSSSTD | |
| | IAFGGGTEVVVK | |
| DDR1-3K | ELVLTQTPASVSEPVGGTVTIKCQASQTISSWLSWYQQKPGQPPKLLIY | 121 |
| | YAFNLASGVPSRFKGSGSGTEFTLTISDLECADAATYYCQQGISSSNVD | |
| | NVFGGGTEVVVK | |
| DDR1-5K | ELVLTQTPASVSEPVGGTVTIKCQASQTISSWLSWYQQKPGQPPKLLIY | 122 |
| | YAFNLASGVPSRFKGSGSGTEYTLTISDLECADAATYYCQCTYGSGSSS | |
| | SYGCAFGGGTELEIK | |
| DDR1-6K | ELVMTQTPSPVSAAVGGTVTISCQSSQSVYSNYLSWYQQKPGQPPKLLI | 123 |
| | YETSTLASGVPSRFKGSGSGTQFTLTISDVQCDDAATYYCQGGYSEIIE | |
| | NTFGGGTEVEIK | |
| DDR1-9K | ELVMTQTPASVEAAVGGTVTIKCQASQSIGSVLAWYQQKPGQRPKLLIS | 124 |
| | GVFDLASGVPSRFKGSGSGTEFTLTISDLECADAATYYCQYIPYGSSPF | |
| | GGGTEVVVK | |
| DDR1-11K | ELVMTQTASPVSAAVGGTVTINCQASQSIGSTYLSWYQQKPGQPPKLLI | 125 |
| | YKASILASGVPSRFSGSGSGTEYTLTISGVQCDDAATYYCLYGGFGSST | |
| | GDAFGGGTVLVVK | |
| DDR1-12K | ELVLTQTPASVSEPVGGTVTIKCQASQTIYSNLAWYQQKPGQRPKLLIY | 126 |
| | QASKLASGVPSRFKGSGSGTEYTLTISDLECADAATYYCQSYYGADDYT | |
| | FGGGTEVVVK | |
| DDR1-13K | ELVMTQTPSPVSAAVGGTVSISCQSSKSVYNNNALSWFQQKPGQPPKVL | 127 |
| | IYGVSTLDSGVSSRFSGSGYGTEFTLTISDVQCDDAATYYCAGDYSDIS | |
| | DNNFGGGTELEIK | |
| DDR1-14K | ELDMTQTPASVSEPVGGTVTIKCQASQSISSYLAWYQQKPGQPPKRLIF | 128 |
| | EASTLASGVPSRFSGSGSGTDFTLTISDLECADAATYYCQNNNGFSGSN | |
| | FNNFGGGTEVEIK | |
| DDR1-15K | ELVMTQTPASVEVAVGGTVTIKCQASQTIYSSLAWYQQKPGQPPKLLIY | 129 |
| | KASTLASGVPSRFKGSGSGTQFTLTISGVQCDDAATYYCQQGSSISNVD | |
| | KNAFGGGTEVEIK | |
| DDR1-17K | ELVLTQTPASVSEPVGGTVTIKCQASQSIGSYLSWYQQKAGQPPKRLIY | 130 |
| | EASTLASGVPSRFSGSGSGTDFTLTISDLECADVATYYCQNNNGMTVSD | |
| | FNAFGGGTEVEIK | |
| DDR1-20K | ELDLTQTPASVSAAVGGTVTINCQSSQIIDHDHLSWYQQKPGQRPKLLI | 131 |
| | YRASTLTSGVPSRFKGSGSGTDFTLTISDLECADVATYYCQNNNGMTVS | |
| | DENAFGGGTEVEIK | |
| DDR1-21K | ELVLTQTPSSTSAAVGGTVTISCQSSQSVVDKNWLAWYQQKPGQPPKLL | 132 |
| | IYEASKLASGVPPRFSGSGSGTQFTLTISGVQCDDAATYYCAGDFESGV | |
| | SGFGGGTEVEIK | |
| DDR1-22K | ELVLTQTPSPVSAAVGGTVTINCQSSKNIYNNNALSWFQQKPGQPPKLL | 133 |
| | IYGASTLASGVPSRFKGSGSGTQFTLTISDVQCDDAATYYCAADYSDIS | |
| | DNNFGGGTEVVVK | |
| DDR1-23K | ELVLTQTPSSVSAAVGGTVTISCQSSQSVYSNNYLAWYQQKPGQPPKLL | 134 |
| | IYAASTLASGVPSRFKGSGSGTQFTLTISGVQCDDAAVYYCLGGYNDDA | |
| | NFGGGTEVEIK | |
| DDR1-26K | ELDLTQTPSSVSAAVGGTVTISCQSSESVYSNNHLAWYQQKPGQPPKLL | 135 |
| | IYAASTLASGVPSRFSGSGSGTQFTLTISGVQCDDAAVYYCLGGYNDDA | |
| | NFGGGTEVVVK | |
| DDR1-28K | ELDLTQTPASVEAAVGGTVTIKCQASQSIDNNDLAWYQQKPGQPPNLLI | 136 |
| | SRTSTLASGVSSRFKGSGSGTEFTLTISDLECADAATYYCQSYCVNTYG | |
| | YTFGGGTEVVVK | |
| DDR1-29K | ELVMTQTPASVEAAVGGTVTIKCQASQSISNHLGWYQQKPGQPPKLLIY | 137 |
| | RASTLESGVSSRFKGSGSGSEFTLTISDLECADAATYYCQSYYIINRSN | |
| | YANSFGGGTEVEIK | |
| DDR1-32K | ELVMTQTPASVEAAVGGTVTIKCQASESINSWLAWYQQKPGQRPKLLIY | 138 |
| | DASKLASGVPSRFKGSGSGTQFTLTISDLECADAATYYCQSYYIINRSN | |
| | YGNSFGGGTEVEIK | |
| DDR1-34K | ELDLTQTPASVSAAVGGTVTISCQSSENLYKDNYLAWYQQKPGQPP | 139 |
| | KLLIYGASNLASGVPSRFKGSGSGTQFTLTISDLECDDAATYYCAG | |
| | GYDSVVDFGGGTEVVVK | |

TABLE 8
Heavy chain variable region amino acid sequences of anti-DDR1 antibodies.

| mAb Name | Heavy chain variable amino acid sequences | SEQ ID NO: |
|---|---|---|
| DDR1-1H | QSVEESGGRLVTPGTPLTLTCTVSGFSLSRYAMTWVRQAPGKGLEW | 140 |
| | IGIIGSSGLTYFATWAKGRFTISKTSTTVDLKITSPTTEDTATYFC | |
| | ARGMWYDDSDDYEDYFNLWGPGTLVTISS | |
| DDR1-3H | QSVKESGGRLVTPGTPLTLTCTVSGIDLSSYAMSWVRQAPGKGLEW | 141 |
| | IGTINIGGGTWDATWARGRFTISRTSTTVDLKITSPTIGDTATYFC | |
| | ARDVDAHTLTYFTLWGPGTLVTISS | |
| DDR1-5H | QSVKESGGRLVTPGTPLTLTCTVSGFTLSNNAISWVRQAPGKGLEW | 142 |
| | IGIIYASGRTYYATWAKGRFTISKTSTTVDLKMTSPTTEDTATYFC | |
| | ARGDTETDYGIPYFDLWGPGTLVTISS | |
| DDR1-6H | SQSLKESGGDLVKPGASRTLTCIAPGFSFSSSYYMCWVRQAPGKGL | 143 |
| | EWIACIYASSGSTYYASWAKGRFTISKTSSTTVTLQMTTLTAADTA | |
| | TYFCAAILGADYRLTRLDLWGQGTLVTVSS | |
| DDR1-9H | QSLEESGGRLVTPGTPLTLTCTASGFSLNRYYMLWVRQAPGEGLEW | 144 |
| | IGTISYGDTTYYASWAKGRFTISKTSTTVDLKMTSPTTEDTATYFC | |
| | ARADTGDNGYLGLQLWGPGTLVTVSS | |
| DDR1-11H | QSLEESGGDLVKPGASLTLTCTASGFSFSSGYYMCWVRQAPGKGLE | 145 |
| | WIACIYTGRTDFTDYASWAKGRFTISKTSSTTVTLQLTTLTAADTA | |
| | TYFCARGDYSGGVGGNYWLDLWGQGTLVTISS | |
| DDR1-12H | QSLEESGGRLVTPGTPLTLTCTVSGIDLSNTWMNWVRQAPGKGLEW | 146 |
| | IGVITDSGTTYYANWAKGRFTISRTSTTVDLKMPSLTTEDTATYFC | |
| | GRDPGDITSGTNDLWGPGTLVTISS | |
| DDR1-13H | EQSVEESGGRLVTPGGSLTLTCTASGFSLNNYAIIWVRQAPGKGLE | 147 |
| | YIGIFNNGDIYYANWAKGRFTISKTSTTVGLKIVSPTTEDTATYFC | |
| | ARTGYRTGGWLWGPGTLVTISS | |
| DDR1-14H | QSVKESGGRLVTPGTPLTLTCTVSGIDLSYYAMSWVRQAPGKGLEY | 148 |
| | IGIINGRGDTGYATWAKGRFTISKTSTTVDLRITSPTIEDTATYFC | |
| | AREDSAIPFIVGNYYGMDLWGPGTLVTVSS | |
| DDR1-15H | SQSLEESGGDLVKPGASLTLTCTASTFSFNSRYWTCWVRQAPGKGL | 149 |
| | EWIGCINNGDISTYYASWATGRFTISKSSSTTVTLHMTSLTAADTA | |
| | TYFCAKGGNLAGDCYGLWGPGTLVTISS | |
| DDR1-17H | SSVEESGGRLVAPGTPLTLTCTVSGFSLNRYAMSWVRQAPGKGLEW | 150 |
| | IGIIGSSGSTYYASWAKGRFTISKTSTTVDLKITSPTTEDTATYFC | |
| | ARDLDDSYGYTYATGMDIRLDLWGQGTLVTVSS | |
| DDR1-20H | QSVKESGGGLFKPMDTLTLTCTVSGFSLSDYAMSWVRQAPGKGLEW | 151 |
| | IGIINSRDDTGYASWAKGRFTISKTSSTTVDLRITSPTTEDTATYF | |
| | CAREDSSIPFIVGNYYGMDLWGPGTLVTVSS | |
| DDR1-21H | QSLEESGGRLVTPGTPLTLTCTVSGFSLSSYGVHWVRQAPGKGLDW | 152 |
| | IGKIYPSGSIYYSSWAKGRFTISKTSTTVDLKMTSLTTEDTATYFC | |
| | VRYLTGSSDLHLWGPGTLVTISS | |
| DDR1-22H | QSVKESGGRLVTPGGSLTLTCTVSGFSLSDYAMIWVRQAPGKGLEY | 153 |
| | IGIINNGDIYYATWAKGRFTISETSSTTMGLNIISPTTEDTATYFC | |
| | ARPGYRTGIWLWGPGTLVTISS | |
| DDR1-23H | SQSVKESGGDLVKPGASLTLTCKASGFDLRSYYYMCWVRQAPGKGL | 154 |
| | EWIACIHGGEGNTYYASWAKGRFTISKTSSTAVTLQMTSLTAADTA | |
| | TYFCARGGWTNYFWGPGTLVTVSS | |
| DDR1-26H | EQSLKESGGDLVKPGASLTLTCTASGFDLSSNYYMCWVRQAPGKGP | 155 |
| | EWIACIYSSNTRTWYARWAKGRFTISKTSSTAVTLQMTSLTAADTA | |
| | TYFCARGGWTNYLWGPGTLVTISS | |
| DDR1-28H | QSVEESGGRLVTPGTPLTLTCTVSGFSLSSHDMIWVRQAAGKGLEW | 156 |
| | IGLIISSGNTWYASWAKGRFTISKTSTTVDLKMTSLTTEDTATYFC | |
| | ARDVYSGASPWGPGTLVTISS | |
| DDR1-29H | QSVKSGGGLVKPGASLTLTCKASTFSFNSRYWTCWVRQAPGKGLEW | 157 |
| | IGCINNGDITTYYTNWATGRFTISKSSSTTVTLQMTSLTAADTATY | |
| | FCAKGGNLAGDCYGLWGPGTLVTISG | |
| DDR1-32H | QSLEESGGRLVTPGTPLTLTCTASGFSLSSYYMSWVRQAPGEGLEW | 158 |
| | IGTITTAGPLYYATWAKGRFTISKTSTTVDLKMTGPTTEDTATYFC | |
| | ARGHAGSIYYSYFDLWGPGTLVTVSS | |
| DDR1-34H | QSVKESGGGLVKPEGSLTLTCKASGFDLSSYYYMCWVRQAPGKGLE | 159 |
| | WIACIYTSSGATWYANWAKGRFTISKTSSTTVTLQMTALTAADTAT | |
| | YFCARGGWCDFNLWGPGTLVTISS | |

In some embodiments, the anti-DDR1 antibody or antigen-binding fragment thereof comprises a light chain variable region having a light chain variable region amino acid sequence selected from SEQ ID NOs: 120-139, and a heavy chain variable region amino acid sequence selected from SEQ ID NOs: 140-159. In various embodiments any one of the variable light chain amino acid sequences corresponding to SEQ ID NOs: 120-139 can be used in combination with any one of the variable heavy chain amino acid sequences corresponding to SEQ ID NOs: 140-159.

In some embodiments, the anti-DDR1 antibody or antigen-binding fragment thereof comprises a light chain variable region having an amino acid sequence of SEQ ID NO: 120 (DDR1-1K) and a heavy chain variable region having an amino acid sequence of SEQ ID NO: 140 (DDR1-1H).

In some embodiments, the anti-DDR1 antibody or antigen-binding fragment thereof comprises a light chain variable region having an amino acid sequence of SEQ ID NO: 121 (DDR1-3K) and a heavy chain variable region having an amino acid sequence of SEQ ID NO: 141 (DDR1-3H).

In some embodiments, the anti-DDR1 antibody or antigen-binding fragment thereof comprises a light chain variable region having an amino acid sequence of SEQ ID NO: 122 (DDR1-5K) and a heavy chain variable region having an amino acid sequence of SEQ ID NO: 142 (DDR1-5H).

In some embodiments, the anti-DDR1 antibody or antigen-binding fragment thereof comprises a light chain variable region having an amino acid sequence of SEQ ID NO: 123 (DDR1-6K) and a heavy chain variable region having an amino acid sequence of SEQ ID NO: 143 (DDR1-6H).

In some embodiments, the anti-DDR1 antibody or antigen-binding fragment thereof comprises a light chain variable region having an amino acid sequence of SEQ ID NO: 124 (DDR1-9K) and a heavy chain variable region having an amino acid sequence of SEQ ID NO: 144 (DDR1-9H).

In some embodiments, the anti-DDR1 antibody or antigen-binding fragment thereof comprises a light chain variable region having an amino acid sequence of SEQ ID NO: 125 (DDR1-11K) and a heavy chain variable region having an amino acid sequence of SEQ ID NO: 145 (DDR1-11H).

In some embodiments, the anti-DDR1 antibody or antigen-binding fragment thereof comprises a light chain variable region having an amino acid sequence of SEQ ID NO: 126 (DDR1-12K) and a heavy chain variable region having an amino acid sequence of SEQ ID NO: 146 (DDR1-12H).

In some embodiments, the anti-DDR1 antibody or antigen-binding fragment thereof comprises a light chain variable region having an amino acid sequence of SEQ ID NO: 127 (DDR1-13K) and a heavy chain variable region having an amino acid sequence of SEQ ID NO: 147 (DDR1-13H).

In some embodiments, the anti-DDR1 antibody or antigen-binding fragment thereof comprises a light chain variable region having an amino acid sequence of SEQ ID NO: 128 (DDR1-14K) and a heavy chain variable region having an amino acid sequence of SEQ ID NO: 148 (DDR1-14H).

In some embodiments, the anti-DDR1 antibody or antigen-binding fragment thereof comprises a light chain variable region having an amino acid sequence of SEQ ID NO: 129 (DDR1-15K) and a heavy chain variable region having an amino acid sequence of SEQ ID NO: 149 (DDR1-15H).

In some embodiments, the anti-DDR1 antibody or antigen-binding fragment thereof comprises a light chain variable region having an amino acid sequence of SEQ ID NO: 130 (DDR1-17K) and a heavy chain variable region having an amino acid sequence of SEQ ID NO: 150 (DDR1-17H).

In some embodiments, the anti-DDR1 antibody or antigen-binding fragment thereof comprises a light chain variable region having an amino acid sequence of SEQ ID NO: 131 (DDR1-20K) and a heavy chain variable region having an amino acid sequence of SEQ ID NO: 151 (DDR1-20H).

In some embodiments, the anti-DDR1 antibody or antigen-binding fragment thereof comprises a light chain variable region having an amino acid sequence of SEQ ID NO: 132 (DDR1-21K) and a heavy chain variable region having an amino acid sequence of SEQ ID NO: 152 (DDR1-21H).

In some embodiments, the anti-DDR1 antibody or antigen-binding fragment thereof comprises a light chain variable region having an amino acid sequence of SEQ ID NO: 133 (DDR1-22K) and a heavy chain variable region having an amino acid sequence of SEQ ID NO: 153 (DDR1-22H).

In some embodiments, the anti-DDR1 antibody or antigen-binding fragment thereof comprises a light chain variable region having an amino acid sequence of SEQ ID NO: 134 (DDR1-23K) and a heavy chain variable region having an amino acid sequence of SEQ ID NO: 154 (DDR1-23H).

In some embodiments, the anti-DDR1 antibody or antigen-binding fragment thereof comprises a light chain variable region having an amino acid sequence of SEQ ID NO: 135 (DDR1-26K) and a heavy chain variable region having an amino acid sequence of SEQ ID NO: 155 (DDR1-26H).

In some embodiments, the anti-DDR1 antibody or antigen-binding fragment thereof comprises a light chain variable region having an amino acid sequence of SEQ ID NO: 136 (DDR1-28K) and a heavy chain variable region having an amino acid sequence of SEQ ID NO: 156 (DDR1-28H).

In some embodiments, the anti-DDR1 antibody or antigen-binding fragment thereof comprises a light chain variable region having an amino acid sequence of SEQ ID NO: 137 (DDR1-29K) and a heavy chain variable region having an amino acid sequence of SEQ ID NO: 157 (DDR1-29H).

In some embodiments, the anti-DDR1 antibody or antigen-binding fragment thereof comprises a light chain variable region having an amino acid sequence of SEQ ID NO: 138 (DDR1-32K) and a heavy chain variable region having an amino acid sequence of SEQ ID NO: 158 (DDR1-32H).

In some embodiments, the anti-DDR1 antibody or antigen-binding fragment thereof comprises a light chain variable region having an amino acid sequence of SEQ ID NO: 139 (DDR1-34K) and a heavy chain variable region having an amino acid sequence of SEQ ID NO: 159 (DDR1-34H).

The individual CDRs of an antibody disclosed herein can be determined according to any CDR numbering scheme known in the art.

In some embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In some embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226, all of which are herein incorporated by reference in their entireties).

In some embodiments, one or more of the CDRs of an antibody disclosed herein can be determined according to MacCallum R M et al., (1996) J Mol Biol 262:732-745, herein incorporated by reference in its entirety. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001), herein incorporated by reference in its entirety.

In some embodiments, the CDRs of an antibody disclosed herein can be determined according to the IMGT numbering system as described in: Lefranc M-P, (1999) The Immunologist 7:132-136; Lefranc M-P et al., (1999) Nucleic Acids Res 27:209-212, each of which is herein incorporated by reference in its entirety; and Lefranc M-P et al., (2009) Nucleic Acids Res 37: D1006-D1012.

In some embodiments, the CDRs of an antibody disclosed herein can be determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety.

In some embodiments, the CDRs of an antibody disclosed herein can be determined according to the AHo numbering system, as described in Honegger and Plückthun A. J. Mol. Biol. 309:657-670 (2001), herein incorporated by reference in its entirety.

In some embodiments, the individual CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of DDR1.

In some embodiments, the anti-DDR1 antibody or antigen-binding fragments thereof is a variant, wherein the light chain variable region sequence and/or the heavy chain variable region sequence of the variant has 1, 2, 3, 4, 5, 6, 7, 8, 9, 10 or more amino acid substitutions, additions, deletions, or combinations of compared to the parent light chain variable region sequence or the heavy chain variable region sequence, wherein the variant retains the binding specificity to the DDR1 protein and/or other functional properties. In some embodiments, the light chain variable region sequence and/or the heavy chain variable region sequence of the variant has 1, 2, 3, 4, 5, 6, 7, 8, 9, 10 or more conservative or non-conservative amino acid substitutions. In some embodiments, the variant has 1, 2 or 3 amino acid substitutions, additions, deletions, or combinations of in one or more of the CDRLs and/or CDRHs of the variant light chain variable region or the variant heavy chain variable region as compared to the parent CDRLs or CDRHs. In some embodiments, the variant antibody or antigen-binding fragment thereof has 1, 2, 3, 4, 5, 6, 7, 8, 9, or 10 amino acid substitutions, additions, deletions, or combinations thereof, in the framework region sequences of the light chain variable region and/or heavy chain variable region compared to the parent light chain variable region sequence or the heavy chain variable region sequence. In some embodiments, the antibody or antigen-binding fragment thereof has 1, 2, 3, 4, 5, 6, 7, 8, 9, 10 or more conservative or non-conservative amino acid substitutions in the framework region sequences of the light chain variable region and/or heavy chain variable region. The foregoing variations apply to each of the light chain variable regions and heavy chain variable regions as shown in Table 3, Table 7, and Table 8.

In some embodiments, the instant disclosure provides an isolated antibody that specifically binds to DDR1 comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 3, 11, or 12. In an embodiment, the instant disclosure provides an isolated antibody that specifically binds to DDR1 comprising a VL comprising an amino acid sequence set forth in SEQ ID NO: 3, 11, or 12. In an embodiment, the amino acid sequence of the VL consists of the amino acid sequence set forth in SEQ ID NO: 3, 11, or 12.

In some embodiments, the instant disclosure provides an isolated antibody that specifically binds to DDR1 comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 3. In an embodiment, the instant disclosure provides an isolated antibody that specifically binds to DDR1 comprising a VL comprising an amino acid sequence set forth in SEQ ID NO: 3. In an embodiment, the amino acid sequence of the VL consists of the amino acid sequence set forth in SEQ ID NO: 3.

In some embodiments, the instant disclosure provides an isolated antibody that specifically binds to DDR1 comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 11. In an embodiment, the instant disclosure provides an isolated antibody that specifically binds to DDR1 comprising a VL comprising an amino acid sequence set forth in SEQ ID NO: 11. In an embodiment, the amino acid sequence of the VL consists of the amino acid sequence set forth in SEQ ID NO: 11.

In some embodiments, the instant disclosure provides an isolated antibody that specifically binds to DDR1 comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 12. In an embodiment, the instant disclosure provides an isolated antibody that specifically binds to DDR1 comprising a VL comprising an amino acid sequence set forth in SEQ ID NO: 12. In an embodiment, the amino acid sequence of the VL consists of the amino acid sequence set forth in SEQ ID NO: 12.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 4. In an embodiment, the instant disclosure provides an isolated antibody that specifically binds to DDR1 comprising a VH comprising an amino acid sequence set forth in SEQ ID NO: 4. In an embodiment, the amino acid sequence of the VH consists of the amino acid sequence set forth in SEQ ID NO: 4.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 13. In an embodiment, the instant disclosure provides an isolated antibody that specifically binds to DDR1 comprising a VH comprising an amino acid sequence set forth in SEQ ID NO: 13. In an embodiment, the amino acid sequence of the VH consists of the amino acid sequence set forth in SEQ ID NO: 13.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 4 or 13, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 3, 11, or 12. In an embodiment, the instant disclosure provides an isolated antibody that specifically binds to DDR1, comprising a VH comprising an amino acid sequence of SEQ ID NO: 4 or 13, and a VL comprising an amino acid sequence of SEQ ID NO: 3, 11, or 12. In an embodiment, the amino acid sequence of the VH consists of the amino acid sequence set forth in SEQ ID NO: 4 or 13; and the amino acid sequence of the VL consists of the amino acid sequence set forth in SEQ ID NO: 3, 11, or 12.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 4, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 3. In an embodiment, the instant disclosure provides an isolated antibody that specifically binds to DDR1, comprising a VH comprising an amino acid sequence of SEQ ID NO: 4, and a VL comprising an amino acid sequence of SEQ ID NO: 3. In an embodiment, the amino acid sequence of the VH consists of the amino acid sequence set forth in SEQ ID NO: 4; and the amino acid sequence of the VL consists of the amino acid sequence set forth in SEQ ID NO: 3.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 13, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 11. In an embodiment, the instant disclosure provides an isolated antibody that specifically binds to DDR1, comprising a VH comprising an amino acid sequence of SEQ ID NO: 13, and a VL comprising an amino acid sequence of SEQ ID NO: 11. In an embodiment, the amino acid sequence of the VH consists of the amino acid sequence set forth in SEQ ID NO: 13; and the amino acid sequence of the VL consists of the amino acid sequence set forth in SEQ ID NO: 11.

In an embodiment, the instant disclosure provides an isolated antibody that specifically binds to DDR1 comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 13, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 12. In an embodiment, the instant disclosure provides an isolated antibody that specifically binds to DDR1, comprising a VH comprising an amino acid sequence of SEQ ID NO: 13, and a VL comprising an amino acid sequence of SEQ ID NO: 12. In an embodiment, the amino acid sequence of the VH consists of the amino acid sequence set forth in SEQ ID NO: 13; and the amino acid sequence of the VL consists of the amino acid sequence set forth in SEQ ID NO: 12.

In an embodiment, the instant disclosure provides an isolated antibody that specifically binds to DDR1 comprising the VH and VL amino acid sequences set forth in SEQ ID NOs: 3 and 4; 11 and 13; or 12 and 13, respectively. In an embodiment, the amino acid sequences of VH and VL consist of the amino acid sequences set forth in SEQ ID NOs: 3 and 4; 11 and 13; or 12 and 13, respectively.

In an embodiment, the instant disclosure provides an isolated antibody that cross-competes for binding to DDR1 with an antibody comprising the VH and VL amino acid sequences set forth in SEQ ID NOs: 3 and 4; 11 and 13; or 12 and 13, respectively.

In an embodiment, the instant disclosure provides an isolated antibody that binds to the same or an overlapping epitope of DDR1 as an antibody described herein, e.g., an antibody comprising the VH and VL amino acid sequences set forth in SEQ ID NOs: 3 and 4; 11 and 13; or 12 and 13, respectively.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 120. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 140. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 120, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 140.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 121. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 141. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 121, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 141.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 122. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 142. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 122, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 142.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 123. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 143. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 123, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 143.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 124. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 144. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 124, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 144.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 125. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 145. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 125, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 145.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 126. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 146. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 126, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 146.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 127. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 147. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 127, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 147.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 128. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 148. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 128, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 148.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 129. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 149. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 129, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 149.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 130. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 150. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 130, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 150.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 131. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 151. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 131, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 151.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 132. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 152. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 132, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 152.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 133. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 153. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 133, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 153.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 134. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 154. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 134, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 154.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 135. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 155. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 135, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 155.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 136. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 156. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 136, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 156.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 137. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 157. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 137, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 157.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 138. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 158. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 138, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 158.

In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 139. In some embodiments, the anti-DDR1 antibody or antigen-binding fragments thereof comprises a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 159. In some embodiments, the anti-DDR1 antibody or antigen-binding fragments thereof comprises a VL amino acid sequence having at least 80%, 85%, 90%, 91%, 92%, 93%, 94%, 95%, 96%, 97%, 98%, 99%, or 100% sequence identity to the VL amino acid sequence of SEQ ID NO: 139, and a VH amino acid sequence having at least 80%, 85%, 90%, 91%, 92%, 93%, 94%, 95%, 96%, 97%, 98%, 99%, or 100% sequence identity to the VH amino acid sequence of SEQ ID NO: 159.

In an embodiment, the epitope of an antibody can be determined by, e.g., NMR spectroscopy, surface plasmon resonance (BIAcore™), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303, all of which are herein incorporated by reference in their entireties). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed. Carter C W; Roversi P et al., (2000) Acta Crystallogr D Biol Crystallogr 56 (Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In an embodiment, the epitope of an antibody is determined using alanine scanning mutagenesis studies. In addition, or antibodies that recognize and bind to the same or overlapping epitopes of DDR1 (e.g., human DDR1 or mouse DDR1) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as DDR1 (e.g., human DDR1 or mouse DDR1). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9:242-253); solid phase direct biotin-avidin EIA (see Kirkland T N et al., (1986) J Immunol 137:3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25 (1): 7-15); solid phase direct biotin-avidin EIA (see Cheung R C et al., (1990) Virology 176:546-52); and direct labeled RIA (see Moldenhauer G et al., (1990) Scand J Immunol 32:77-82), all of which are herein incorporated by reference in their entireties. Typically, such an assay involves the use of purified antigen (e.g., DDR1, such as human DDR1 or mouse DDR1) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, e.g., Wagener C et al., (1983) J Immunol 130:2308-2315; Wagener C et al., (1984) J Immunol Methods 68:269-274; Kuroki M et al., (1990) Cancer Res 50:4872-4879; Kuroki M et al., (1992) Immunol Invest 21:523-538; Kuroki M et al., (1992) Hybridoma 11:391-407 and Antibodies: A Laboratory Manual, ed. Harlow E & Lane D editors supra, pp. 386-389, all of which are herein incorporated by reference in their entireties.

In an embodiment, the antibody inhibits the binding of human DDR1 to human collagen. In an embodiment, the binding of human DDR1 to human collagen is reduced by more than 50%, 55%, 60%, 65%, 70%, 75%, 80%, 85%, 90%, 95%, 96%, 97%, 98%, or 99% in the presence of the antibody relative to the binding of human DDR1 to human collagen in the absence of the antibody.

In an embodiment, the antibody disclosed herein is conjugated to a cytotoxic agent, cytostatic agent, toxin, radionuclide, or detectable label. In an embodiment the cytotoxic agent is able to induce death or destruction of a cell in contact therewith. In an embodiment, the cytostatic agent is able to prevent or substantially reduce proliferation and/or inhibits the activity or function of a cell in contact therewith. In an embodiment, the cytotoxic agent or cytostatic agent is a chemotherapeutic agent. In an embodiment, the radionuclide is selected from the group consisting of the isotopes ³H, ¹⁴C, ³²p, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. In an embodiment, the detectable label comprises a fluorescent moiety or a click chemistry handle.

Any immunoglobulin (Ig) constant region can be used in the antibodies disclosed herein. In an embodiment, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In an embodiment, one, two or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In an embodiment, one, two or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In an embodiment, one, two or more amino acid mutations (e.g., substitutions, insertions or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046; 6,121,022; 6,277,375; and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions or deletions) are introduced into an IgG constant region, or FcRn-binding fragment thereof (preferably an Fc or hinge-Fc fragment) to increase the half-life of the antibody in vivo. In an embodiment, the antibodies may have one or more amino acid mutations (e.g., substitutions) in the second constant (CH2) domain (residues 231-340 of human IgG1) and/or the third constant (CH3) domain (residues 341-447 of human IgG₁), numbered according to the EU numbering system. In an embodiment, the constant region of the IgG₁ of antibody described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall'Acqua W F et al., (2006) J Biol Chem 281:23514-24, which is herein incorporated by reference in its entirety). In certain embodiments, an antibody comprises an IgG constant region comprising one, two, three or more amino acid substitutions of amino acid residues at positions 251-257, 285-290, 308-314, 385-389, and 428-436, numbered according to the EU numbering system.

In certain embodiments, one, two or more mutations (e.g., amino acid substitutions) are introduced into an Fc region (e.g., a CH2 domain (residues 231-340 of human IgG1) and/or a CH3 domain (residues 341-447 of human IgG1, numbered according to the EU numbering system) and/or a hinge region (residues 216-230, numbered according to the EU numbering system)) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In an embodiment, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG₁, a variant human IgG₂, or a variant human IgG₄ heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E and L328F; and V264E, S267E and L328F, according to the EU numbering system. In an embodiment, the FcγRIIB is expressed on a cell selected from the group consisting of macrophages, monocytes, B cells, dendritic cells, endothelial cells, and activated T cells.

In an embodiment, one, two or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In an embodiment, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276:6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; an A330L substitution; an I332E substitution; or a P396L substitution, numbered according to the EU numbering system. In some embodiments, the following mutations are made in the constant region of an antibody: L234A, L235E, G237A, A330S, and P331S. In some embodiments, the following mutations are made in the constant region of an antibody: P329G, L234A and L235A.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, I332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In an embodiment, an antibody described herein comprises the constant region of an IgG₁ with an N297Q or N297A amino acid substitution, numbered according to the EU numbering system. In certain embodiments, an antibody described herein comprises the constant region of an IgG₁ with a mutation selected from the group consisting of D265A, P329A, and a combination thereof, numbered according to the EU numbering system. In another embodiment, an antibody described herein comprises the constant region of an IgG₁ with a mutation selected from the group consisting of L234A, L235A, and a combination thereof, numbered according to the EU numbering system. In another embodiment, an antibody described herein comprises the constant region of an IgG₁ with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235 and D265 in a human IgG1 heavy chain, numbered according to the EU numbering system, are not L, L and D, respectively. This approach is described in detail in International Publication No. WO 14/108483, which is herein incorporated by reference in its entirety. In an embodiment, the amino acids corresponding to positions L234, L235 and D265 in a human IgG1 heavy chain are F, E and A; or A, A and A, respectively, numbered according to the EU numbering system.

In an embodiment, the amino acids at positions 433, 434 and 436 of the heavy chain constant region, according to the EU numbering system, are K, F and Y, respectively. In an embodiment, the amino acids at positions 252, 254 and 256 of the heavy chain constant region, according to the EU numbering system, are Y, T and E, respectively. In an embodiment, the amino acids at positions 428 and 434 of the heavy chain constant region, according to the EU numbering system, are L and S, respectively. In an embodiment, the amino acid at positions 309, 311 and 434 of the heavy chain constant region, according to the EU numbering system, are D, H and S, respectively.

In an embodiment, one or more amino acids selected from amino acid residues 329, 331 and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In an embodiment, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In an embodiment, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In an embodiment, any of the constant region mutations or modifications described herein can be introduced into one or both heavy chain constant regions of an antibody described herein having two heavy chain constant regions.

In an embodiment, the instant disclosure provides an isolated antibody that specifically binds to DDR1 and functions as an antagonist (e.g., decreases or inhibits DDR1 activity).

In an embodiment, the instant disclosure provides an isolated antibody that specifically binds to DDR1 and decreases or inhibits DDR1 activity by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein and/or known to one of skill in the art, relative to DDR1 activity without any antibody or with an unrelated antibody (e.g., an antibody that does not specifically bind to DDR1). In an embodiment, the instant disclosure provides an isolated antibody that specifically binds to DDR1 and decreases or inhibits DDR1 activity by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more, as assessed by methods described herein and/or known to one of skill in the art, relative to DDR1 activity without any antibody or with an unrelated antibody (e.g., an antibody that does not specifically bind to DDR1). Non-limiting examples of DDR1 activity can include DDR1 signaling; DDR1 binding to collagen (e.g., collagen I, II, III, IV, or V); or DDR1 phosphorylation. In an embodiment, a decrease in a DDR1 activity is assessed as described in the Examples.

In an embodiment, the instant disclosure provides an isolated antibody that specifically binds to DDR1 and decreases or inhibits DDR1 phosphorylation by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein and/or known to one of skill in the art, relative to DDR1 phosphorylation without any antibody or with an unrelated antibody (e.g., an antibody that does not specifically bind to DDR1). In an embodiment, the instant disclosure provides an isolated antibody that specifically binds to DDR1 and decreases or inhibits DDR1 phosphorylation by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more, as assessed by methods described herein and/or known to one of skill in the art, relative to DDR1 phosphorylation without any antibody or with an unrelated antibody (e.g., an antibody that does not specifically bind to DDR1).

In an embodiment, the instant disclosure provides an isolated antibody that specifically binds to DDR1 and decreases or inhibits DDR1 binding to collagen by at least 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein and/or known to one of skill in the art, relative to DDR1 binding to collagen without any antibody or with an unrelated antibody (e.g., an antibody that does not specifically bind to DDR1). In an embodiment, the instant disclosure provides an isolated antibody that specifically binds to DDR1 and decreases or inhibits DDR1 binding to collagen by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or more, as assessed by methods described herein and/or known to one of skill in the art, relative to DDR1 binding to collagen without any antibody or with an unrelated antibody (e.g., an antibody that does not specifically bind to DDR1).

In an embodiment, the instant disclosure provides an isolated antibody that specifically binds DDR1 with a dissociation constant (K_{D}) value of less than 10 nM, less than 5 nM, less than 2 nM, less than 1 nM, less than 0.5 nM, or less than 0.1 nM.

### 7.3 Pharmaceutical Compositions

Provided herein are compositions comprising an isolated anti-DDR1 antibody disclosed herein having the desired degree of purity in a physiologically acceptable carrier, excipient or stabilizer (see, e.g., Remington's Pharmaceutical Sciences (1990) Mack Publishing Co., Easton, PA). Acceptable carriers, excipients or stabilizers are nontoxic to recipients at the dosages and concentrations employed, and include buffers such as phosphate, citrate and other organic acids; antioxidants, including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine, or lysine; monosaccharides, disaccharides and other carbohydrates, including glucose, mannose or dextrins; chelating agents such as EDTA; sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™ or polyethylene glycol (PEG).

In an embodiment, pharmaceutical compositions comprise an isolated anti-DDR1 antibody disclosed herein, and optionally one or more additional prophylactic or therapeutic agents, in a pharmaceutically acceptable carrier. In an embodiment, the antibody is the only active ingredient included in the pharmaceutical composition. In an embodiment, the instant disclosure provides a pharmaceutical composition comprising an isolated anti-DDR1 antibody disclosed herein for use as a medicament. In another embodiment, the instant disclosure provides a pharmaceutical composition for use in a method for the treatment of a DDR1 related disease. In some embodiments, the DDR1 related disease is cancer or a fibrotic condition.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents, and other pharmaceutically acceptable substances. Examples of aqueous vehicles include Sodium Chloride Injection, Ringer's Injection, Isotonic Dextrose Injection, Sterile Water Injection, and Dextrose and Lactated Ringer's Injection. Nonaqueous parenteral vehicles include fixed oils of vegetable origin, cottonseed oil, corn oil, sesame oil, and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles, and sodium hydroxide, hydrochloric acid, citric acid, or lactic acid for pH adjustment.

A pharmaceutical composition may be formulated for any route of administration to a subject. Specific examples of routes of administration include intranasal, oral, pulmonary, transdermal, intradermal, and parenteral. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances, such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol and polypropylene glycol, and mixtures thereof.

Topical mixtures comprising an antibody are prepared as described for the local and systemic administration. The resulting mixture can be a solution, suspension, emulsion or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

An isolated anti-DDR1 antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983; 6,261,595; 6,256,533; 6,167,301; 6,024,975; 6,010,715; 5,985,317; 5,983,134; 5,948,433; and 5,860,957, all of which are herein incorporated by reference in their entireties.

In an embodiment, a pharmaceutical composition comprising antibody described herein is a lyophilized powder, which can be reconstituted for administration as solutions, emulsions and other mixtures. It may also be reconstituted and formulated as solids or gels. The lyophilized powder is prepared by dissolving antibody described herein, or a pharmaceutically acceptable derivative thereof, in a suitable solvent. In an embodiment, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or another suitable agent. The solvent may also contain a buffer, such as citrate, sodium or potassium phosphate, or other such buffer known to those of skill in the art at, in certain embodiments, about neutral pH. Subsequent sterile filtration of the solution followed by lyophilization under standard conditions known to those of skill in the art provides the desired formulation. In an embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The isolated anti-DDR1 antibodies disclosed herein, and other compositions provided herein can also be formulated to be targeted to a particular tissue, receptor or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6, 120, 751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In an embodiment, an antibody described herein is targeted to a tumor.

The compositions to be used for in vivo administration can be sterile. This is readily accomplished by filtration through, e.g., sterile filtration membranes.

### 7.4 Polynucleotides, Vectors and Methods of Producing Antibodies

In an aspect, provided herein are polynucleotides comprising a nucleotide sequence encoding an antibody, or a portion thereof, described herein or a fragment thereof (e.g., a VL and/or VH; and a light chain and/or heavy chain) that specifically binds to DDR1 antigen, and vectors, e.g., vectors comprising such polynucleotides for recombinant expression in host cells (e.g., E. coli and mammalian cells). Provided herein are polynucleotides comprising nucleotide sequences encoding a heavy and/or light chain of an antibody provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular, less than about 10%) of other material, e.g., cellular material, culture medium, other nucleic acid molecules, chemical precursors and/or other chemicals. In an embodiment, a nucleic acid molecule(s) encoding an antibody described herein is isolated or purified.

In an aspect, provided herein are polynucleotides comprising nucleotide sequences encoding antibodies, which specifically bind to DDR1 polypeptide and comprise an amino acid sequence as described herein, as well as antibodies which compete with such antibodies for binding to DDR1 polypeptide (e.g., in a dose-dependent manner), or which bind to the same epitope as that of such antibodies.

In an aspect, provided herein are polynucleotides comprising a nucleotide sequence encoding the light chain or heavy chain of antibody described herein. The polynucleotides can comprise nucleotide sequences encoding a light chain comprising the VL FRs and CDRs of antibodies described herein (see, e.g., Tables 2-8) or nucleotide sequences encoding a heavy chain comprising the VH FRs and CDRs of antibodies described herein (see, e.g., Tables 2-8). In an embodiment, a polynucleotide encodes a VH, VL, heavy chain, and/or light chain of an antibody described herein. In an embodiment, a polynucleotide encodes the first VH and the first VL of an antibody described herein. In an embodiment, a polynucleotide encodes the second VH and the second VL of an antibody described herein. In an embodiment, a polynucleotide encodes the first heavy chain and the first light chain of an antibody described herein. In an embodiment, a polynucleotide encodes the second heavy chain and the second light chain of an antibody described herein. In an embodiment, a polynucleotide encodes the VH and/or the VL, or the heavy chain and/or the light chain, of an isolated antibody described herein.

In some embodiments, a polynucleotide encoding the heavy chain and/or the light chain, of an isolated antibody described herein further encodes one or more signal peptide. In some embodiments, the signal peptide comprises a secretion signal peptide. In some embodiments, the secretion signal peptide comprises an immunoglobulin secretion signal peptide. Exemplary signal peptides include, but are not limited to, heavy chain IgM, IgG, IgD, IgA, and IgE signal peptides, and light chain kappa and lambda signal peptides. In certain embodiments, the signal peptide is a mammalian signal peptide. In certain embodiments, the signal peptide is a human signal peptide. In certain embodiments, the signal peptide comprises a rodent e.g., murine or lagomorph, e.g., rabbit signal peptide. In an embodiment, the signal peptide is a primate (e.g., non-human primate) signal peptide.

Also provided herein are polynucleotides encoding an isolated anti-DDR1 antibody that is optimized, e.g., by codon/RNA optimization, replacement with heterologous signal sequences, and elimination of mRNA instability elements. Methods to generate optimized nucleic acids encoding an isolated anti-DDR1 antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid. Such methods can increase expression of an isolated anti-DDR1 antibody or fragment thereof by at least 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an isolated anti-DDR1 antibody encoded by polynucleotides that have not been optimized.

In an embodiment, an optimized polynucleotide sequence encoding an isolated anti-DDR1 antibody described herein or a fragment thereof (e.g., VL domain and/or VH domain) can hybridize to an antisense (e.g., complementary) polynucleotide of an unoptimized polynucleotide sequence encoding an isolated anti-DDR1 antibody described herein or a fragment thereof (e.g., VL domain and/or VH domain). In an embodiment, an optimized nucleotide sequence encoding an isolated anti-DDR1 antibody described herein or a fragment thereof, hybridizes under high stringency conditions to an antisense polynucleotide of an unoptimized polynucleotide sequence encoding an isolated anti-DDR1 antibody described herein or a fragment thereof. In an embodiment, an optimized nucleotide sequence encoding an isolated anti-DDR1 antibody described herein, or a fragment thereof hybridizes under high stringency, intermediate or lower stringency hybridization conditions to an antisense polynucleotide of an unoptimized nucleotide sequence encoding an isolated anti-DDR1 antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Tables 2-8, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17:242-6, herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antigen-binding region of an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning.

If a clone containing a nucleic acid encoding a particular antigen-binding region or antibody is not available, but the sequence of the antigen-binding region or antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding isolated anti-DDR1 antibodies described herein can be readily isolated and sequenced using conventional procedures (e.g., by using oligonucleotide probes that are capable of binding specifically to genes encoding the heavy and light chains of the anti-DDR1). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-DDR1 antibodies in the recombinant host cells.

To generate whole antibodies or antigen-binding regions, PCR primers, including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In an embodiment, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and has been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In an aspect, provided herein are cells (e.g., host cells) expressing (e.g., recombinantly) antibodies described herein which specifically bind to DDR1, and related polynucleotides and expression vectors. Provided herein are vectors (e.g., expression vectors) comprising polynucleotides comprising nucleotide sequences encoding anti-DDR1 antibodies or a fragment for recombinant expression in host cells, preferably in mammalian cells (e.g., CHO cells). Also provided herein are host cells comprising such vectors for recombinantly expressing anti-DDR1 antibodies described herein (e.g., human or humanized antibody). In an aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to DDR1 generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties), and variable regions of the antibody can be cloned into such a vector for expression of the entire heavy, the entire light chain, or both the entire heavy and light chains.

In an embodiment, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding containing an antibody described herein or fragments thereof, or a heavy or light chain thereof, or fragment thereof, or a single chain antibody described herein, operably linked to a promoter for expression of such sequences in the host cell.

In an embodiment, a host cell comprises a polynucleotide encoding the VH and VL of an isolated antibody described herein. In another embodiment, a host cell comprises a vector comprising a polynucleotide encoding the VH and VL of an isolated antibody described herein. In another embodiment, a host cell comprises a first polynucleotide encoding the VH of an isolated antibody described herein, and a second polynucleotide encoding the VL of an isolated antibody described herein. In another embodiment, a host cell comprises a first vector comprising a first polynucleotide encoding the VH of an isolated antibody described herein, and a second vector comprising a second polynucleotide encoding the VL of an isolated antibody described herein.

In an embodiment, a heavy chain/heavy chain variable region expressed by a first host cell is associated with a light chain/light chain variable region of a second host cell to form an anti-DDR1 antibody as described herein. In an embodiment, provided herein is a population of host cells comprising such first host cell and such second host cell.

In an embodiment, provided herein is a population of vectors comprising a first vector comprising a polynucleotide encoding a light chain/light chain variable region of an anti-DDR1 antibody described herein, and a second vector comprising a polynucleotide encoding a heavy chain/heavy chain variable region of an anti-DDR1 antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA, or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COSI or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing antibodies described herein are Chinese hamster ovary (CHO) cells, for example CHO cells from the CHO GS System™ (Lonza). In an embodiment, the heavy chain and/or light chain of an antibody produced by a CHO cell may have an N-terminal glutamine or glutamate residue replaced by pyroglutamate. In an embodiment, cells for expressing antibodies described herein are human cells, e.g., human cell lines. In an embodiment, a mammalian expression vector is pOptiVEC™ or pcDNA3.3. In an embodiment, bacterial cells such as E. coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus is an effective expression system for antibodies (Foecking M K & Hofstetter H (1986) Gene 45:101-5; and Cockett M I et al., (1990) Biotechnology 8 (7): 662-7, each of which is herein incorporated by reference in its entirety). In an embodiment, antibodies described herein are produced by CHO cells or NS0 cells. In an embodiment, the expression of nucleotide sequences encoding antibodies described herein which specifically bind to DDR1 is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HEK-293T, HepG2, SP210, R1.1, B-W, L-M, BSC1, BSC40, YB/20, BMT10, and HsS78Bst cells. In an embodiment, anti-DDR1 antibodies described herein are produced in mammalian cells, such as CHO cells.

In an embodiment, the antibodies described herein have reduced fucose content or no fucose content. Such antibodies can be produced using techniques known to one skilled in the art. For example, the antibodies can be expressed in cells deficient or lacking the ability to fucosylate. In an example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-DDR1 antibody described herein can be engineered. In an embodiment, a cell provided herein stably expresses a light chain/light chain variable region and a heavy chain/heavy chain variable region which associate to form an antigen-binding region, or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for one to two days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method can advantageously be used to engineer cell lines which express an anti-DDR1 described herein or a fragment thereof. Such engineered cell lines can be particularly useful in the screening and evaluation of compositions that interact directly or indirectly with the antibody molecule.

A number of selection systems can be used, including but not limited to the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al., (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of an antibody molecule can be increased by vector amplification (for a review, see, Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of the host cell will increase the number of copies of the marker gene. Since the amplified region is associated with the gene of interest, production of the protein will also increase (Crouse G F et al., (1983) Mol Cell Biol 3:257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322:562-565; and Köhler G (1980) PNAS 77:2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In an embodiment, an antibody described herein is isolated or purified. In an embodiment, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In an embodiment, antibodies described herein are isolated or purified.

Anti-DDR1 antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates); Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In an embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-DDR1 antibody comprising culturing a cell or host cell described herein. In an embodiment, the method is performed in vitro. In an aspect, provided herein is a method of making an anti-DDR1 antibody comprising expressing (e.g., recombinantly expressing) the antibody using a cell or host cell described herein (e.g., a cell or a host cell comprising polynucleotides encoding an antibody described herein). In an embodiment, the cell is an isolated cell. In an embodiment, the exogenous polynucleotides have been introduced into the cell. In an embodiment, the method further comprises the step of purifying the antibody obtained from the cell or host cell.

In an embodiment, an isolated antibody is produced by expressing in a cell a polynucleotide encoding the VH and VL of an antibody described herein under suitable conditions so that the polynucleotides are expressed, and the antibody is produced. In another embodiment, an isolated antibody is produced by expressing in a cell a polynucleotide encoding the heavy chain and light chain of an antibody described herein under suitable conditions so that the polynucleotides are expressed, and the antibody is produced. In an embodiment, an isolated antibody is produced by expressing in a cell a first polynucleotide encoding the VH of an antibody described herein, and a second polynucleotide encoding the VL of an antibody described herein, under suitable conditions so that the polynucleotides are expressed, and the antibody is produced. In an embodiment, an isolated antibody is produced by expressing in a cell a first polynucleotide encoding the heavy chain of an antibody described herein, and a second polynucleotide encoding the light chain of an antibody described herein, under suitable conditions so that the polynucleotides are expressed, and the antibody is produced.

Methods for producing polyclonal antibodies are known in the art (see, for example, Chapter 11 in: Short Protocols in Molecular Biology, (2002) 5th Ed., Ausubel F M et al., eds., John Wiley and Sons, New York, which is herein incorporated by reference in its entirety).

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In an embodiment, a “monoclonal antibody,” as used herein, is an antibody produced by a single cell (e.g., hybridoma or host cell producing a recombinant antibody), wherein the antibody specifically binds to anti-DDR1 as determined, e.g., by ELISA or other antigen-binding or competitive binding assay known in the art or in the examples provided herein. In an embodiment, a monoclonal antibody can be a chimeric antibody or a humanized antibody. In an embodiment, a monoclonal antibody is a monovalent antibody or multivalent (e.g., bivalent) antibody. In an embodiment, a monoclonal antibody is a monospecific or multispecific antibody (e.g., bispecific antibody). Monoclonal antibodies described herein can, for example, be made by the hybridoma method as described in Kohler G & Milstein C (1975) Nature 256:495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce, or are capable of producing, antibodies that will specifically bind to the protein used for immunization (e.g., DDR1). Alternatively, lymphocytes may be immunized in vitro. Lymphocytes then are fused with myeloma cells using a suitable fusing agent, such as polyethylene glycol, to form a hybridoma cell (Goding J W (ed.), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In an embodiment, mice (or other animals, such as rats, monkeys, donkeys, pigs, sheep, hamster, or dogs) can be immunized with an antigen (e.g., DDR1) and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In an embodiment, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin and thymidine (HAT medium), which substances prevent the growth of HGPRT-deficient cells.

In an embodiment, myeloma cells are employed that fuse efficiently, support stable high-level production of antibody by the selected antibody-producing cells, and are sensitive to a medium such as HAT medium. Among these myeloma cell lines are murine myeloma lines, such as the NS0 cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133:3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against DDR1. The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (ed.), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize DDR1, and can be generated by any technique known to those of skill in the art. For example, Fab and F(ab′)2 fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli, and the E. coli is infected with helper phage. Phage used in these methods are typically filamentous phage, including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; International Publication Nos. WO 90/02809; WO 91/10737; WO 92/01047; WO 92/18619; WO 93/11236; WO 95/15982; WO 95/20401; and WO 97/13844; and U.S. Pat. Nos. 5,698,426; 5,223,409; 5,403,484; 5,427,908; 5,516,637; 5,571,698; 5,580,717; 5,658,727; 5,733,743; 5,780,225; 5,821,047; and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′) 2 fragments can also be employed using methods known in the art such as those disclosed in PCT Publication No. WO 92/22324; Mullinax R L et al., (1992) BioTechniques 12 (6): 864-9; Sawai H et al., (1995) Am J Reprod Immunol 34:26-34; and Better M et al., (1988) Science 240:1041-1043, all of which are herein incorporated by reference in their entireties.

In certain embodiments, to generate whole antibodies, PCR primers, including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a non-human mammalian (e.g., mouse, rat, rabbit, etc.) monoclonal antibody fused to a constant region of a human antibody. Methods for producing chimeric antibodies are known in the art. See, e.g., Morrison S L (1985) Science 229:1202-7; Oi V T & Morrison S L (1986) BioTechniques 4:214-221; Gillies S D et al., (1989) J Immunol Methods 125:191-202; and U.S. Pat. Nos. 4,816,397, 4,816,567, 5,807,715, and 6,331,415, all of which are herein incorporated by reference in their entireties.

A humanized antibody is capable of binding to a predetermined antigen, and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CHI, hinge, CH2, CH3, and CH4 regions of the heavy chain. A humanized antibody can be selected from any class of immunoglobulins, including IgM, IgG, IgD, IgA, and IgE, and any isotype, including IgG1, IgG2, IgG3, and IgG4. Humanized antibodies can be produced using a variety of techniques known in the art, including but not limited to, CDR-grafting (European Patent No. EP239400; International Publication No. WO 91/09967; and U.S. Pat. Nos. 5,225,539, 5,530,101, and 5,585,089), veneering or resurfacing (European Patent Nos. EP592106 and EP519596; Padlan E A (1991) Mol Immunol 28 (4/5): 489-498; Studnicka G M et al., (1994) Prot Engineering 7 (6): 805-814; and Roguska M A et al., (1994) PNAS 91:969-973), chain shuffling (U.S. Pat. No. 5,565,332), and techniques disclosed in, e.g., U.S. Pat. Nos. 5,766,886 and 6,407,213, International Publication No. WO 93/17105; Tan P et al., (2002) J Immunol 169:1119-25; Caldas C et al., (2000) Protein Eng. 13 (5): 353-60; Morea V et al., (2000) Methods 20 (3): 267-79; Baca M et al., (1997) J Biol Chem 272 (16): 10678-84; Roguska M A et al., (1996) Protein Eng 9 (10): 895 904; Couto J R et al., (1995) Cancer Res. 55 (23 Supp): 5973s-5977s; Couto J R et al., (1995) Cancer Res 55 (8): 1717-22; Sandhu J S (1994) Gene 150 (2): 409-10; and Pedersen J T et al., (1994) J Mol Biol 235 (3): 959-73, all of which are herein incorporated by reference in their entireties. See also, U.S. Patent Application Publication No. US 2005/0042664 A1, which is herein incorporated by reference in its entirety.

Methods for making multispecific antibodies (e.g., bispecific antibodies) have been described, see, for example, U.S. Pat. Nos. 5,837,242; 5,869,620; 5,989,830; 6,132,992; 7,183,076; 7,951,917; 8,227,577; and 8,586,713, all of which are herein incorporated by reference in their entireties.

Bispecific, bivalent antibodies, and methods of making them, are described, for instance in U.S. Pat. Nos. 5,731,168; 5,807,706; and 5,821,333, and U.S. Patent Application Publication No. 2002/0155537; each of which is herein incorporated by reference in its entirety. Bispecific tetravalent antibodies, and methods of making them are described, for instance, in International Publication Nos. WO 02/096948 and WO 00/44788, the disclosures of both of which are herein incorporated by reference in its entirety. See generally, International Publication Nos. WO 91/00360; WO 92/08802; WO 92/05793; and WO 93/17715; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2008/119353; WO 2011/131746; WO 2011/147986; and WO 2013/060867, and in Labrijn A F et al., (2013) PNAS 110 (13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., International Publication No. WO 2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some embodiments, compositions of the invention have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in some instances, contain IgG₄ and IgG₁, IgG₄ and IgG₂, IgG₄ and IgG₂, IgG₄ and IgG₃, or IgG₁ and IgG₃ chain heterodimers. Such heterodimeric heavy chain antibodies can routinely be engineered by, for example, modifying selected amino acids forming the interface of the CH3 domains in human IgG₄ and the IgG₁ or IgG₃ so as to favor heterodimeric heavy chain formation.

In an embodiment, an antibody described herein, which binds to the same epitope of DDR1 as an anti-DDR1 antibody described herein, is a human antibody. In an embodiment, an antibody described herein, which competitively blocks (e.g., in a dose-dependent manner) any one of the antibodies described herein, from binding to DDR1 is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the Ju region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., DDR1). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13:65-93, herein incorporated by reference in its entirety. For a detailed discussion of this technology for producing human antibodies and human monoclonal antibodies and protocols for producing such antibodies, see, e.g., International Publication Nos. WO 96/33735; WO 96/34096; and WO 98/24893; and U.S. Pat. Nos. 5,413,923; 5,545,806; 5,569,825; 5,625, 126; 5,633,425; 5,661,016; 5,814,318; and 5,939,598, all of which are herein incorporated by reference in their entireties. Examples of mice capable of producing human antibodies include the XenoMouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the TransChromo Mouse™ (Kirin) and the KM Mouse™ (Medarex/Kirin), all of which are herein incorporated by reference in their entireties.

Human antibodies that specifically bind to DDR1 can be made by a variety of methods known in the art, including the phage display methods described above using antibody libraries derived from human immunoglobulin sequences. See also, U.S. Pat. Nos. 4,444,887; 4,716,111; and 5,885,793; and International Publication Nos. WO 91/10741; WO 96/34096; WO 96/33735; WO 98/16654; WO 98/24893; WO 98/46645; and WO 98/50433, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., DDR1). Such methods are known and are described in the art, see, e.g., Shinmoto H et al., (2004) Cytotechnology 46:19-23; Naganawa Y et al., (2005) Human Antibodies 14:27-31, each of which is herein incorporated by reference in its entirety.

### 7.5 Methods of Use

In one aspect, the instant disclosure provides a method of monitoring the effectiveness of an anti-DDR1 antibody in a subject in need thereof. In one embodiment, the method comprises administering an effective amount of the anti-DDR1 antibody to the subject and detecting a level of DDR1 phosphorylation in a sample from the subject. In some embodiments, the anti-DDR1 antibody comprises an anti-DDR1 antibody or a nucleic acid encoding an anti-DDR1 antibody as disclosed herein. In some embodiments, the anti-DDR1 antibody is administered via a suitable route. Non-limiting examples of suitable administration routes include intravenous, oral, parenteral, ophthalmic, pulmonary, and topical administration.

In some embodiments, the level of DDR1 phosphorylation comprises the proportion of total DDR1 in a sample that is phosphorylated at one or more phosphorylation sites. In one embodiment, the one or more phosphorylation site comprises a tyrosine residue. In one embodiment, the tyrosine residue is one known to be autophosphorylated in response to stimulation of the extracellular portion of DDR1. In one embodiment, the tyrosine residue is one known to be autophosphorylated in response to stimulation of the extracellular portion of DDR1 via one or more type of collagen. In some embodiments, the level of DDR1 phosphorylation comprises the absolute quantity of phosphorylated DDR1 proteins in a sample. In some embodiments, the level of DDR1 phosphorylation comprises the absolute quantity of phosphorylated DDR1 sites in a sample.

In some embodiments, the level of DDR1 phosphorylation comprises the proportion of a cleaved form of DDR1 in a sample that is phosphorylated at one or more phosphorylation sites. In some embodiments, the cleaved form of DDR1 has a lower molecular weight relative to the uncleaved form. In some embodiments, the cleaved form has a molecular weight of less than 125 kDa (e.g., less than 120 kDa, less than 115 kDa, less than 110 kDa, less than 105 kDa, less than 100 kDa, less than 95 kDa, less than 90 kDa, less than 85 kDa, less than 80 kDa, less than 75 kDa, less than 70 kDa, less than 65 kDa, less than 60 kDa, less than 55 kDa, less than 50 kDa, less than 45 kDa, less than 40 kDa, less than 35 kDa, less than 30 kDa, less than 25 kDa, less than 20 kDa, less than 15 kDa, less than 10 kDa, or less than 5 kDa). In some embodiments, the cleaved form has a molecular weight of approximately 60 kDa.

A skilled artisan will recognize that any methods of detecting DDR1 phosphorylation can be used with the methods of the present invention. Various methods for detecting phosphorylation are known in the art, including, but not limited to, radioisotope labeling, mass spectrometry, immunoassays with phospho-specific antibodies (e.g., immunoblotting, enzyme-linked immunosorbent assay, intracellular flow cytometry, etc.). A variety of antibodies specific to phosphorylated DDR1 are readily available and known in the art. Exemplary phospho-specific DDR1 antibodies include, but are not limited to, Phospho-DDR1 (Tyr513) (E1N8F) Rabbit mAb #14531 (Cell Signaling Technology), Phospho-DDR1 (Tyr792) Antibody #11994 (Cell Signaling Technology), Phospho-DDR1 (Tyr796) Polyclonal Antibody Phospho-DDR1 (Tyr796) Polyclonal Antibody PA5-106123 (Thermo Fisher Scientific), and Anti-phospho-DDR1 (pTyr513) SAB4504671 (Millipore Sigma).

A skilled artisan will further recognize that any methods of preparing a sample for detecting of DDR1 phosphorylation can be used in the methods of the present invention. As a non-limiting example, a cell or tissue may be lysed by physical (e.g., sonication) and/or chemical (e.g., surfactant) means and processed to remove cellular debris (e.g., centrifugation). In some embodiments, one or more phosphatase inhibitor is included in the sample to prevent premature dephosphorylation of DDR1. In some embodiments, one or more protease inhibitor is included in the sample to prevent premature degradation of DDR1.

In some embodiments, the subject in need thereof has elevated levels of DDR1 phosphorylation in comparison to a reference sample. In some embodiments, the levels of DDR1 phosphorylation are elevated by at least 1%, 5%, 10%, 15%, 20%, 30%, 40%, 50%, 75%, 100%, 150%, 200%, 300%, or 400% in comparison to a reference sample. In some embodiments, the levels of DDR1 phosphorylation are elevated by at least 2-fold, 3-fold, 4-fold, 5-fold, 6-fold, 7-fold, 8-fold, 9-fold, 10-fold, 11-fold, 12-fold, 13-fold, 14-fold, or 15-fold in comparison to a reference sample. In some embodiments, the elevated levels of DDR1 are associated with a disease or disorder. In some embodiments, the elevated levels of DDR1 are a direct result of a disease or disorder. In some embodiments, levels of DDR1 phosphorylation that are elevated by at least 1%, 5%, 10%, 15%, 20%, 30%, 40%, 50%, 75%, 100%, 150%, 200%, 300%, or 400% in comparison to a reference sample are indicative of a disease or disorder. In some embodiments, levels of DDR1 phosphorylation that are elevated by at least 2-fold, 3-fold, 4-fold, 5-fold, 6-fold, 7-fold, 8-fold, 9-fold, 10-fold, 11-fold, 12-fold, 13-fold, 14-fold, or 15-fold in comparison to a reference sample are indicative of a disease or disorder. In some embodiments, the disease or disorder is associated with elevated levels of DDR1 expression (e.g., cancer, fibrosis, etc.). In some embodiments, the disease or disorder is associated with increased binding of DDR1 to collagen. In some embodiments, the disease or disorder is associated with elevated levels of DDR1 phosphorylation. In some embodiments, the disease or disorder is associated with elevated levels of DDR1 expression, binding to collagen, and/or phosphorylation.

In one embodiment, a decrease in DDR1 phosphorylation in the sample from the subject in comparison to a reference sample indicates that the administration of the anti-DDR1 antibody is effective. In one embodiment, a decrease in DDR1 phosphorylation in the sample from the subject in comparison to a reference sample indicates that the administration of the anti-DDR1 antibody is effective for treating a disease or disorder associated with elevated levels of DDR1 phosphorylation. In one embodiment, a decrease in DDR1 phosphorylation in the sample from the subject of at least 1%, 5%, 10%, 15%, 20%, 30%, 40%, 50%, 75%, or 100% in comparison to a reference sample indicates that the administration of the anti-DDR1 antibody is effective for treating a disease or disorder associated with elevated levels of DDR1 phosphorylation. In one embodiment, a decrease in DDR1 phosphorylation in the sample from the subject of at least 0.9-fold, 0.8-fold, 0.7-fold, 0.6-fold, 0.5-fold, 0.4-fold, 0.3-fold, 0.2-fold, or 0.1-fold in comparison to a reference sample indicates that the administration of the anti-DDR1 antibody is effective for treating a disease or disorder associated with elevated levels of DDR1 phosphorylation.

In one aspect, the instant disclosure provides a method of treating a DDR1 related disorder in a subject. In one embodiment, the method comprises administering an effective amount of an anti-DDR1 antibody to the subject and detecting a level of DDR1 phosphorylation in a sample from the subject and a reference sample. In some embodiments, a decrease in DDR1 phosphorylation in the sample from the subject in comparison to the reference sample indicates that the treatment is effective. In one embodiment, a decrease in DDR1 phosphorylation in the sample from the subject of at least 1%, 5%, 10%, 15%, 20%, 30%, 40%, 50%, 75%, or 100% in comparison to the reference sample indicates that the administration of the anti-DDR1 antibody is effective for treating the DDR1 related disease or disorder. In one embodiment, a decrease in DDR1 phosphorylation in the sample from the subject of at least 0.9-fold, 0.8-fold, 0.7-fold, 0.6-fold, 0.5-fold, 0.4-fold, 0.3-fold, 0.2-fold, or 0.1-fold in comparison to the reference sample indicates that the administration of the anti-DDR1 antibody is effective for treating the DDR1 related disease or disorder.

In one embodiment, the method comprises detecting a level of DDR1 phosphorylation in a sample from a subject and administering an effective amount of an anti-DDR1 antibody to the subject if DDR1 phosphorylation in the sample from the subject is higher in comparison to a reference sample. In one embodiment, the effective amount of the anti-DDR1 antibody is administered if the DDR1 phosphorylation in the subject sample is at least 1%, 5%, 10%, 15%, 20%, 30%, 40%, 50%, 75%, 100%, 150%, 200%, 300%, or 400% higher than the reference sample. In one embodiment, the effective amount of the anti-DDR1 antibody is administered if the DDR1 phosphorylation in the subject sample is at least 2-fold, 3-fold, 4-fold, 5-fold, 6-fold, 7-fold, 8-fold, 9-fold, 10-fold, 11-fold, 12-fold, 13-fold, 14-fold, or 15-fold higher than the reference sample.

In one aspect, the instant disclosure provides a method of screening for a subject with a DDR1 related disorder that is likely to be effectively treated with an anti-DDR1 antibody. In one embodiment, the method comprises detecting a level of DDR1 phosphorylation in a sample from the subject, wherein if DDR1 phosphorylation in the sample from the subject is higher in comparison to a reference sample, then the DDR1 related disorder is likely to be effectively treated with an anti-DDR1 antibody. In some embodiments, the DDR1 related disorder is likely to be effectively treated by an anti-DDR1 if the DDR1 phosphorylation in the subject sample is at least 1%, 5%, 10%, 15%, 20%, 30%, 40%, 50%, 75%, 100%, 150%, 200%, 300%, or 400% higher than the reference sample. In some embodiments, the DDR1 related disorder is likely to be effectively treated by an anti-DDR1 if the DDR1 phosphorylation in the subject sample is at least 2-fold, 3-fold, 4-fold, 5-fold, 6-fold, 7-fold, 8-fold, 9-fold, 10-fold, 11-fold, 12-fold, 13-fold, 14-fold, or 15-fold higher than the reference sample. In some embodiments, the DDR1 related disorder comprising elevated DDR1 phosphorylation levels is 1.5 times, 2 times, 3 times, 4 times, 5 times, 6 times, 7 times, 8 times, 9 times, or 10 times more likely to be effectively treated by an anti-DDR1 antibody than a therapeutic that does not specifically target DDR1. In some embodiments, the DDR1 related disorder comprising elevated DDR1 phosphorylation levels is 1.5 times, 2 times, 3 times, 4 times, 5 times, 6 times, 7 times, 8 times, 9 times, or 10 times more likely to be effectively treated by an anti-DDR1 antibody than an antibody that does not specifically target DDR1.

In various embodiments of the methods described herein, the subject sample or the reference sample comprises a subject cell, tissue, biological fluid, or a derivative thereof. In some embodiments, the cell comprises a blood cell, skin cell, a cancer cell or a cell derived from a fibrotic tissue. In some embodiments, the blood cell comprises a red blood cell, a white blood cell, or a platelet. In some embodiments, the white blood cell comprises a monocyte, lymphocyte, neutrophil, eosinophil, basophil, or macrophage. In some embodiments, the tissue comprises a skin tissue, a cancer tissue or a fibrotic tissue. In some embodiments, the skin tissue is gathered via a skin punch biopsy. In some embodiments, the biological fluid comprises blood (e.g., whole blood, plasma, serum, etc.), saliva or sputum. In some embodiments, a derivative of the subject or reference cell or tissue is a lysate. In some embodiments, a derivative of the subject or reference biological fluid is an isolate.

In various embodiments of the methods described herein, the level of DDR1 phosphorylation in the subject sample is compared to the level of DDR1 phosphorylation in a reference sample. Non-limiting examples of reference samples include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In some embodiments, the reference sample comprises a sample of a healthy individual. In some embodiments, the reference sample comprises a sample of a healthy individual obtained after successful treatment for a DDR1 related disease or disorder. In some embodiments, the reference sample comprises a sample of a healthy individual with no known history of having a DDR1 related disease or disorder. In some embodiments, the reference sample comprises a sample of the subject. In some embodiments, the reference sample comprises a sample of the subject obtained prior to developing a DDR1 related disease or disorder. In some embodiments, the reference sample comprises a sample of the subject obtained after developing a DDR1 related disease or disorder.

In one aspect, the instant disclosure provides a method of screening for an anti-DDR1 antibody. In some embodiments, the method comprises administering an effective amount of the anti-DDR1 antibody to a cell and detecting a level of DDR1 phosphorylation in the cell. In one embodiment, the method comprises screening for an anti-DDR1 antibody that is effective in treating a DDR1 related disorder, wherein a decrease in DDR1 phosphorylation in the cell in comparison to a reference cell indicates that the anti-DDR1 antibody is effective in treating cancer. In another embodiment, the method comprises screening for an anti-DDR1 antibody that is effective in reducing collagen interaction with a cell, wherein a decrease in DDR1 phosphorylation in the cell in comparison to a reference cell indicates that the anti-DDR1 antibody is effective in reducing collagen interaction with the cell. Exemplary collagen types include, but are not limited to, collagen I, collagen II, collagen III, collagen IV, collagen V, collagen VI, collagen VII, collagen VIII, collagen IX, collagen X, collagen XI, collagen XII, collagen XIII, collagen XIV, collagen XV, collagen XVI, collagen XVII, collagen XVIII, collagen XIX, collagen XX, collagen XXI, collagen XXII, collagen XXIII, collagen XXIV, collagen XXV, collagen XXVI, collagen XXVII, and collagen XXVIII. In some embodiments, the collagen type comprises collagen I, collagen II, collagen III, or collagen V.

In various embodiments of the methods described herein, the disease or disorder associated with elevated DDR1 phosphorylation (i.e., the DDR1 related disease or disorder) comprises cancer. Exemplary cancer tissues that may associated with elevated DDR1 phosphorylation include, but are not limited to, cancer or cancer cells of the bladder, blood, bone, bone marrow, brain, breast, colon, esophagus, intestine, gum, head, kidney, liver, lung, nasopharynx, neck, ovary, prostate, skin, stomach, pancreas, testis, tongue, cervix, or uterus.

Exemplary histological types of cancer that may associated with elevated DDR1 phosphorylation include, but are not limited to, neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometrial carcinoma; skin appending carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; bookmark ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonic rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonic carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some aspects, the tumor may include osteosarcoma, angiosarcoma, rhabdosarcoma, leiomyosarcoma, Ewing sarcoma, glioblastoma, neuroblastoma, or leukemia.

In various embodiments of the methods described herein, the disease or disorder associated with elevated DDR1 phosphorylation (i.e., the DDR1 related disease or disorder) comprises a fibrotic condition. In some embodiments, the fibrotic condition comprises organ fibrosis. In some embodiments, the fibrotic condition comprises fibrosis of the skin, kidney, liver, lung, or heart. In some embodiments, the fibrotic condition comprises skin hypertrophic scarring, scleroderma, lung scarring, interstitial lung disease, idiopathic pulmonary fibrosis, cirrhotic liver fibrosis, or renal fibrosis.

### 7.6 Kits

Also provided are kits comprising one or more antibodies described herein, or pharmaceutical compositions or conjugates thereof. In an embodiment, provided herein is a pharmaceutical pack or kit comprising one or more containers filled with one or more of the ingredients of the pharmaceutical compositions described herein, such as one or more antibodies provided herein. In an embodiment, the kits contain a pharmaceutical composition described herein and any prophylactic or therapeutic agent, such as those described herein. In an embodiment, the kits may contain a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In an embodiment, a kit comprises an antibody described herein, preferably purified antibody, in one or more containers. In an embodiment, kits described herein contain a substantially isolated DDR1 antigen as a control. In an embodiment, the kits described herein further comprise a control antibody which does not react with DDR1 antigen. In an embodiment, kits described herein contain one or more elements for detecting the binding of an antibody to an DDR1 antigen (e.g., the antibody can be conjugated to a detectable substrate such as a fluorescent compound, an enzymatic substrate, a radioactive compound, or a luminescent compound, or a second antibody which recognizes the first antibody can be conjugated to a detectable substrate). In an embodiment, a kit provided herein can include a recombinantly produced or chemically synthesized DDR1 antigen. The DDR1 antigen provided in the kit can also be attached to a solid support. In an embodiment, the detecting means of the above-described kit includes a solid support to which an DDR I antigen is attached. Such a kit can also include a non-attached reporter-labeled anti-human antibody or anti-mouse/rat antibody. In this embodiment, binding of the antibody to the DDR1 antigen can be detected by binding of the said reporter-labeled antibody. In certain embodiments, the present invention relates to the use of a kit of the present invention for in vitro assaying and/or detecting DDR1 antigen in a biological sample. In an embodiment, a kit provided herein comprises one or more antibody capable of specifically detecting one or more site of phosphorylation of DDR1. In an embodiment, a kit provided herein comprises one or more biomolecule capable of binding to and/or stimulating DDR1 phosphorylation in a cell (e.g., collagen). In an embodiment, a kit provided herein comprises a reference sample and/or cell as described herein.

### 8. EXAMPLES

The examples in this Section (i.e., Section 8) are offered by way of illustration and not by way of limitation.

### 8.1 Example 1: In Vitro Characterization of Anti-DDR1 Monoclonal Antibodies (mAbs)

Anti-DDR1 mAbs were tested for their ability to inhibit Collagen-induced DDR1 phosphorylation, their impact on cell proliferation and cell death in cultured cancer cells, and their effects on Collagen-dependent adhesion of DDR1-overexpressing cells.

### A. Inhibition of Collagen-Induced DDR1 Phosphorylation in T47D Cells

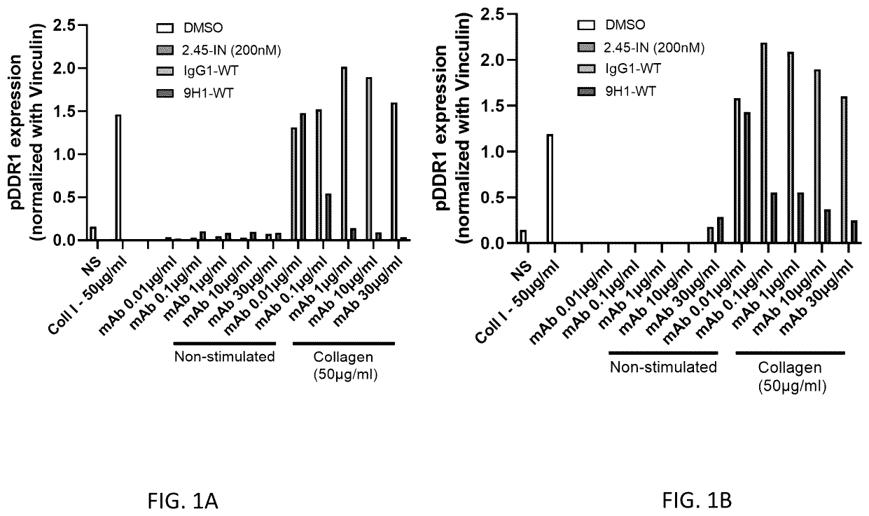

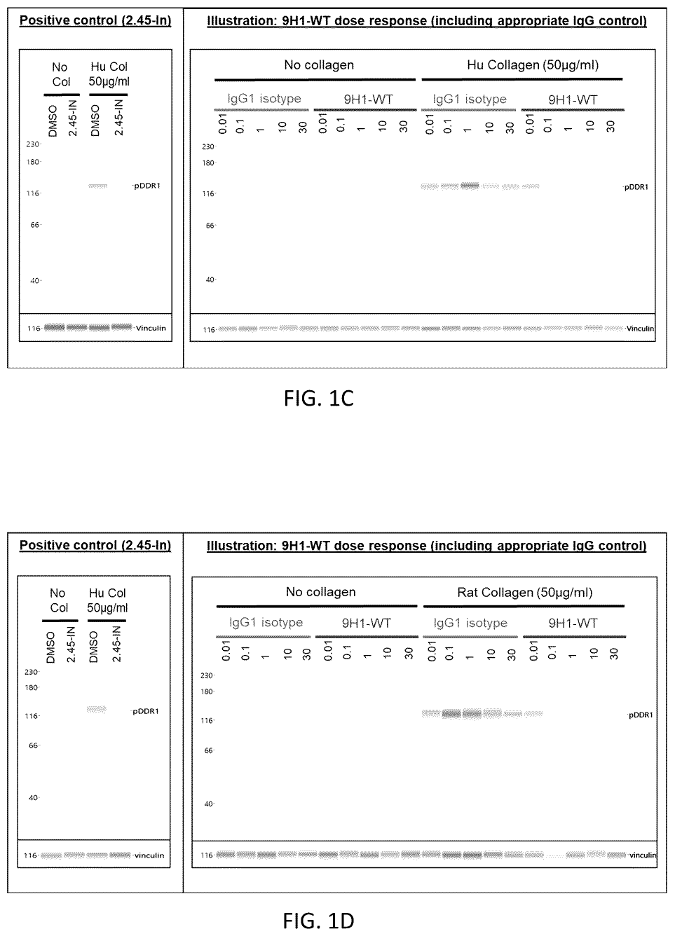

Cultured T47D breast cancer cells were serum starved for 16 hours, pretreated with increasing concentrations of 9H1-WT or IgG1-WT (as a negative control) monoclonal antibodies for two hours, and then treated with 50 μg/ml human or rat Collagen I for 90 minutes. Cells treated with 200 nM of 2.45-IN [2-(4-Bromo-2-oxo-1′-(1H-pyrazolo[4,3-b]pyridine-5-carbonyl)spiro[indoline-3,4′-piperidin]-1-yl)-N-(2,2,2-trifluoroethyl) acetamide] were used as positive control for inhibition of DDR1 phosphorylation. Cell lysate was then analyzed via Jess immunoassay (ProteinSimple). As shown in FIG. 1, 9H1-WT was able to inhibit phosphorylation of DDR1 induced by human Collagen I (FIGS. 1A and 1C) and rat Collagen I (FIGS. 1B and 1D) at concentrations as low as 0.1 μg/ml.

B. Impact of 9H1-WT and Ab #33 mAbs on Cell Proliferation and Cell Death

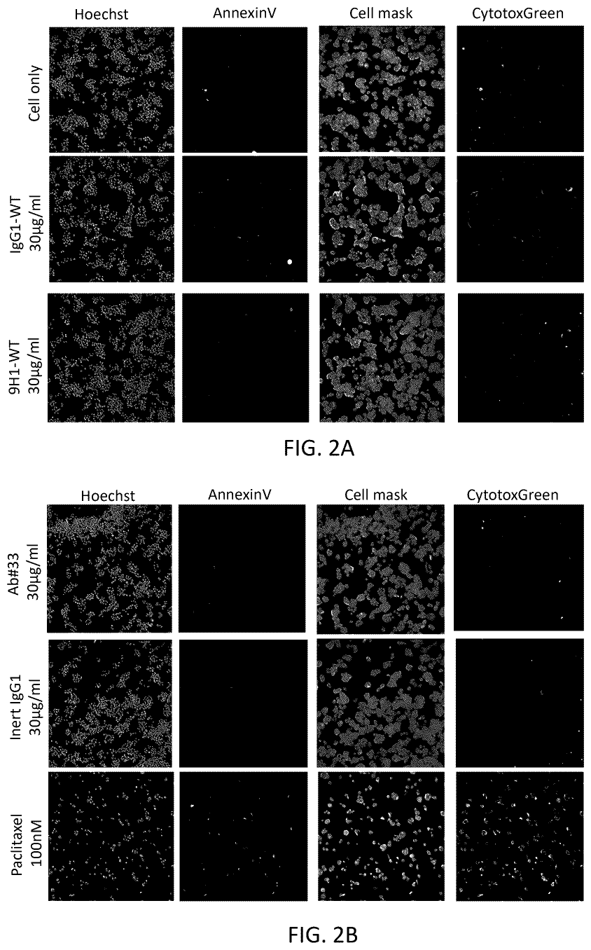

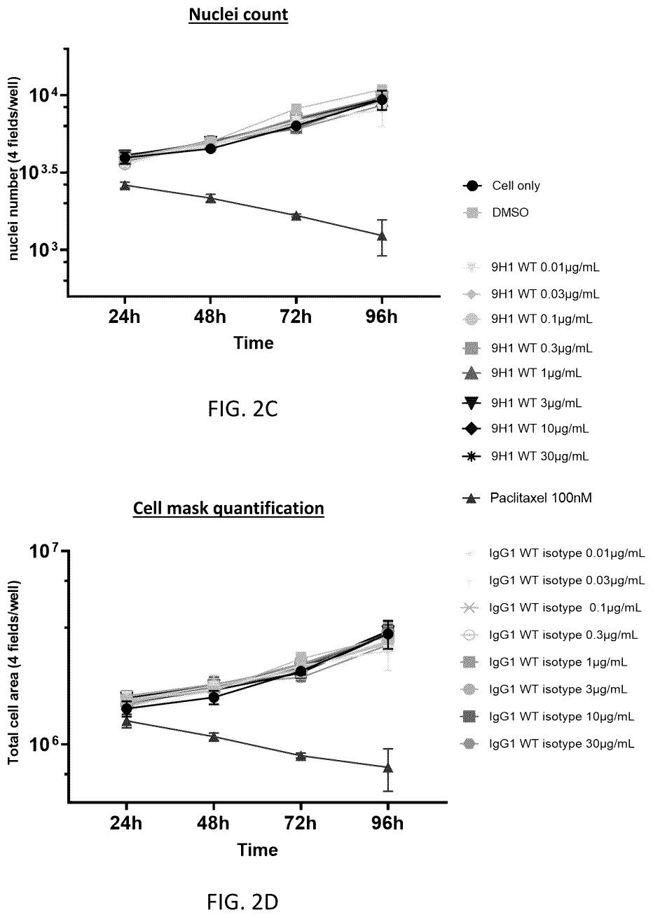

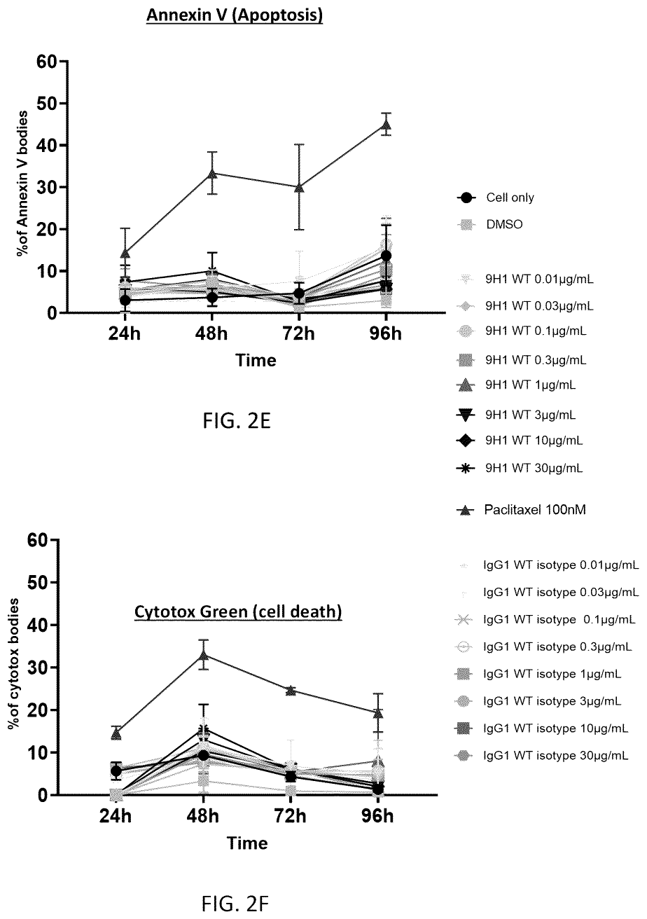

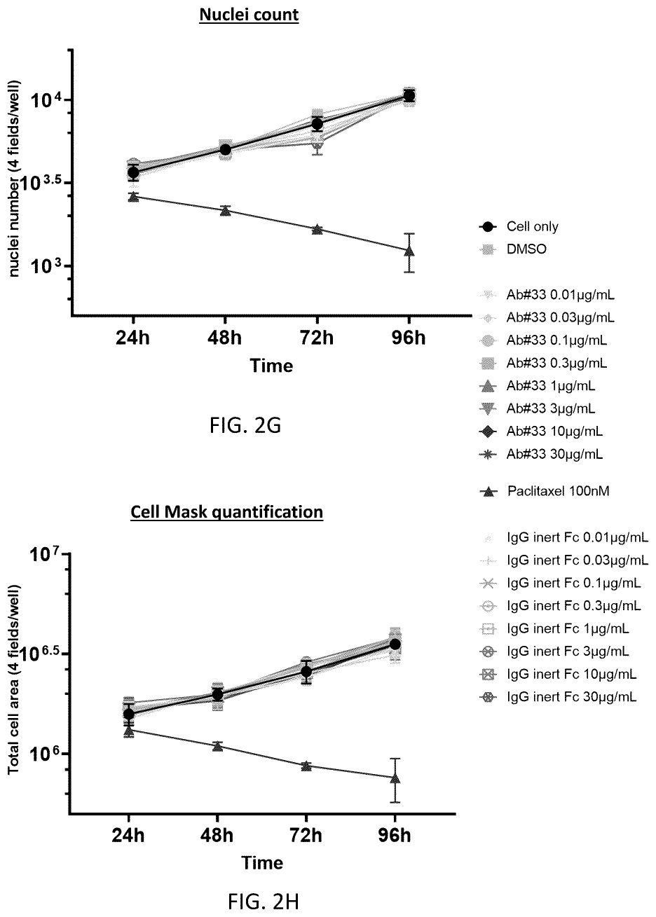

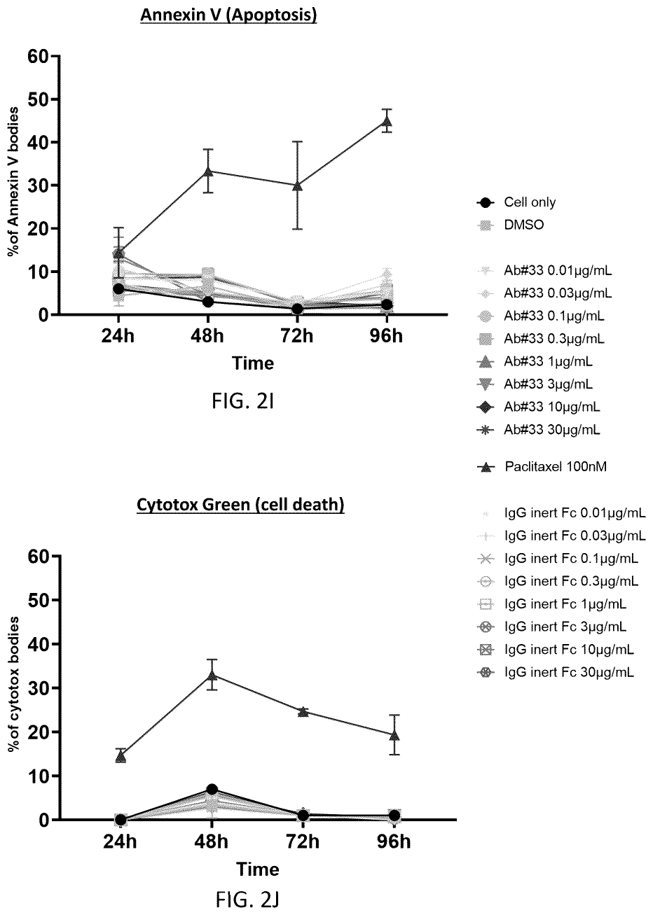

To evaluate the potential cytostatic or cytotoxic effects of 9H1-WT and Ab #33 mAbs, serum-starved T47D cells were pretreated with 9H1-WT or Ab #33, their corresponding IgG controls or the known chemotherapeutic paclitaxel (as a positive control for cell toxicity) and tracked for 96 hours. To measure proliferation, Hoechst staining was used to stain nuclei and CellMask™ (Thermo Fisher Scientific) Deep Red was used to stain cell membranes. To measure cell death, Annexin V was used to stain phosphatidylserine on the cell surface, a marker of apoptosis, and Incucyte® Cytotox Green (Sartorius) to stain cells with compromised membrane integrity, a marker of cell death. As shown in FIG. 2, 9H1-WT had no measurable effect on cell proliferation (FIGS. 2A and 2C-2D) or cell death (FIGS. 2A and 2E-2F) as compared to the DMSO or IgG1 controls, while paclitaxel decreased proliferation and increased cell death, as expected. Similarly, Ab #33 had no measurable effect on cell proliferation (FIGS. 2B and 2G-2H) or cell death (FIGS. 2B and 21-2J), suggesting that 9H1-WT and Ab #33 do not have significant cytostatic or cytotoxic effects.

### C. Collagen Type-Specific Induction of DDR1 Phosphorylation

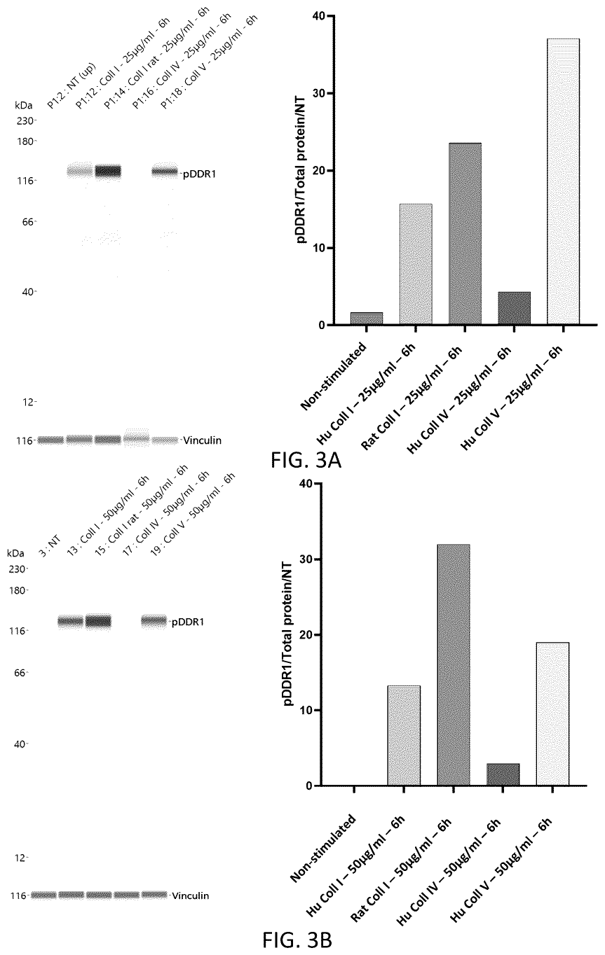

To determine which types of collagen effectively induce DDR1 phosphorylation, serum-starved T47D cells were treated with 25 μg/ml or 50 μg/ml of human or rat collagen I, human collagen IV or human collagen V. As shown in FIGS. 3A and 3B, either rat or human collagen I and human collagen V induced a measurable amount of DDR1 phosphorylation, while human collagen IV did not, suggesting that DDR1 primarily responds to collagen I and collagen V stimulation.

### D. Inhibition of Collagen I- and V-Induced Phosphorylation of DDR1

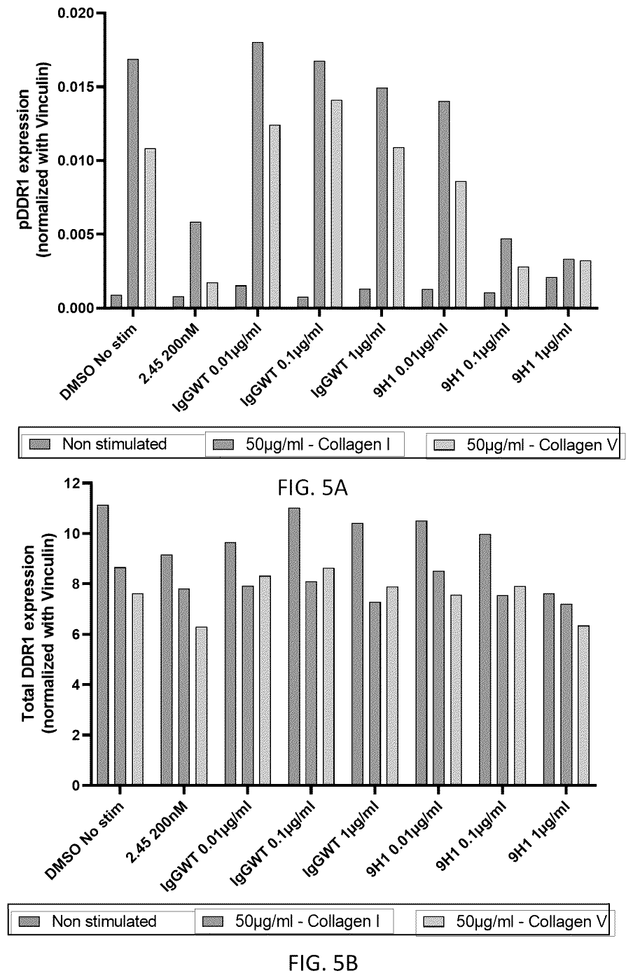

Serum-starved T47D cells were pretreated for two hours with increasing concentrations of 9H1-WT, IgG1-WT as a negative control, or 2.45-IN as a positive control, followed by stimulation with 50 μg/ml of human collagen I or V for 90 minutes. As shown in FIG. 5A, 9H1-WT was able to inhibit both collagen I- and collagen V-induced phosphorylation of DDR1 at a concentration as low as 0.1 μg/ml. FIG. 5B confirms that total DDR1 protein levels were largely unaffected by the treatments.

E. Calculated IC50 for 9H1-WT Inhibition of Collagen I-Induced pDDR1

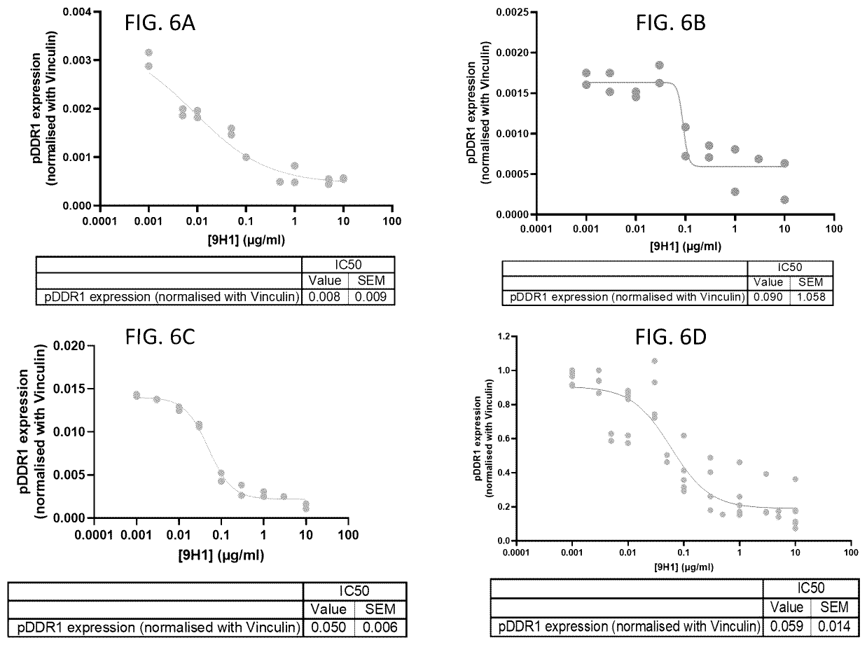

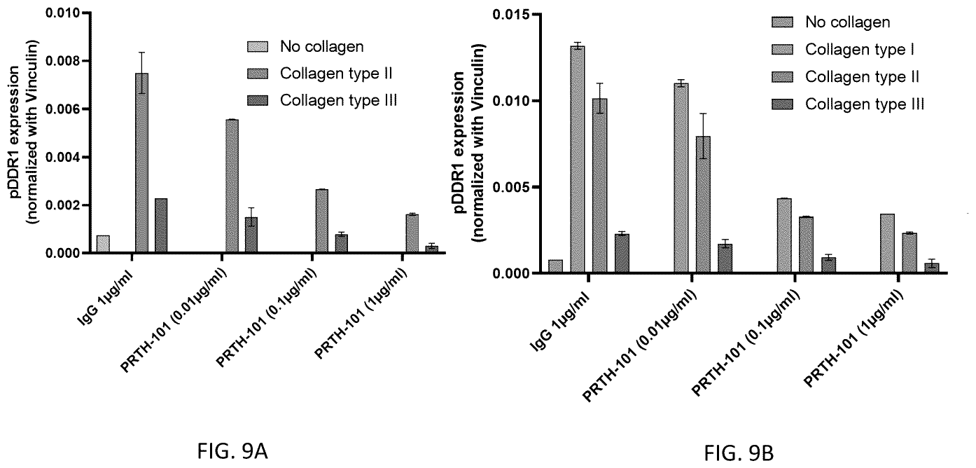

To determine the IC50 of 9H1-WT for inhibiting DDR1 phosphorylation, serum-starved T47D cells were pretreated for two hours with increasing concentrations (on a logarithmic scale) of 9H1-WT, IgG1-WT as a negative control, or 2.45-IN as a positive control, followed by stimulation with 50 μg/ml of human collagen I for 90 minutes. As shown in FIGS. 6A-6D, 9H1-WT (PRTH-101) had an average IC50 of between 0.05-0.06 μg/ml for inhibiting Collagen I-induced phosphorylation of DDR1.

### F. Inhibition of Adhesion of DDR1-Overexpressing (DDR1 OE) Cells to Collagen I

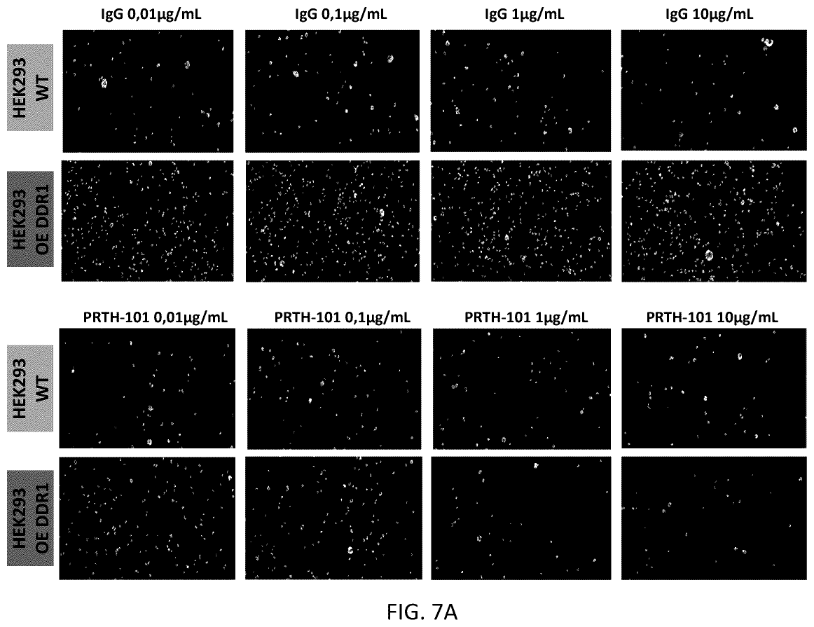

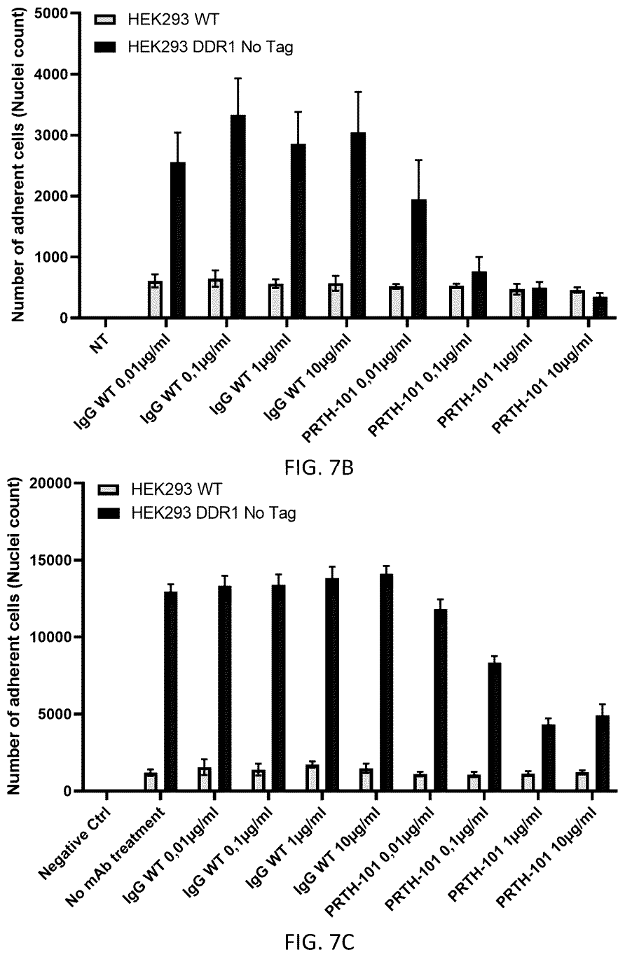

To determine if 9H1-WT is capable of inhibiting DDR1-mediated cell adhesion, HEK293 cells overexpressing DDR1 and WT HEK293 cells were pretreated with increasing concentrations of PRTH-101 or control IgG1-WT, incubated on plates coated with 0.5 μg/cm² collagen I for 30 minutes and stained for nuclei using Hoechst. As shown in FIGS. 7A-7C, DDR1 overexpression results in increased adhesion to collagen I, which is inhibited by 9H1-WT (PRTH-101).

### G. Calculated IC50 for 9H1-WT Inhibition of DDR1 OE Cell Adhesion

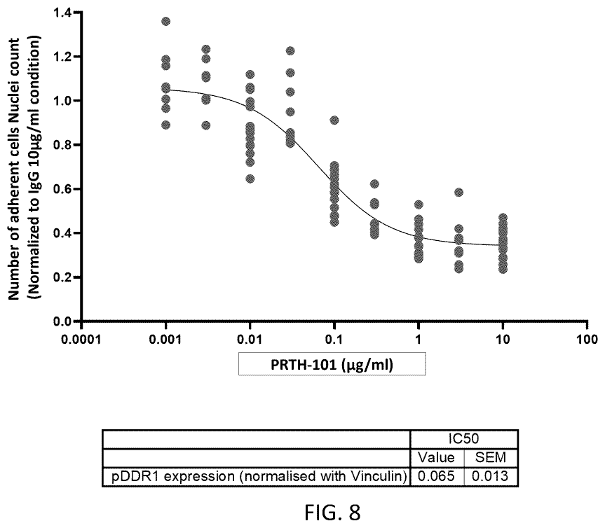

To determine the IC50 for 9H1-WT inhibition of DDR1 OE cell adhesion, HEK293-DDR1 OE cells were pretreated with increasing concentrations (on a logarithmic scale) of 9H1-WT or control IgG1-WT for one hour, incubated on plates coated with 0.5 μg/cm² collagen I for 30 minutes and stained for nuclei using Hoechst. As shown in FIG. 8, the calculated IC50 for 9H1-WT (PRTH-101) of approximately 0.065 μg/ml was comparable to that calculated for collagen I-induced phosphorylation of DDR1 as shown in FIG. 6.

### H. Inhibition of Collagen II- and III-Induced Phosphorylation of DDR1

Serum-starved T47D cells were pretreated for two hours with increasing concentrations of 9H1-WT, or IgG1-WT as a negative control, followed by stimulation with 50 μg/ml of human collagen I, II or III for 90 minutes. As shown in FIG. 9A, collagen II, and to a much lesser extent collagen III, induced DDR1 phosphorylation in T47D, and both were inhibited by 9H1-WT (PRTH-101). FIG. 9B confirms the results of FIG. 9A and displays a similar pattern for collagen I.

I. Rabbit and Chimeric mAb #33-Mediated Inhibition of pDDR1

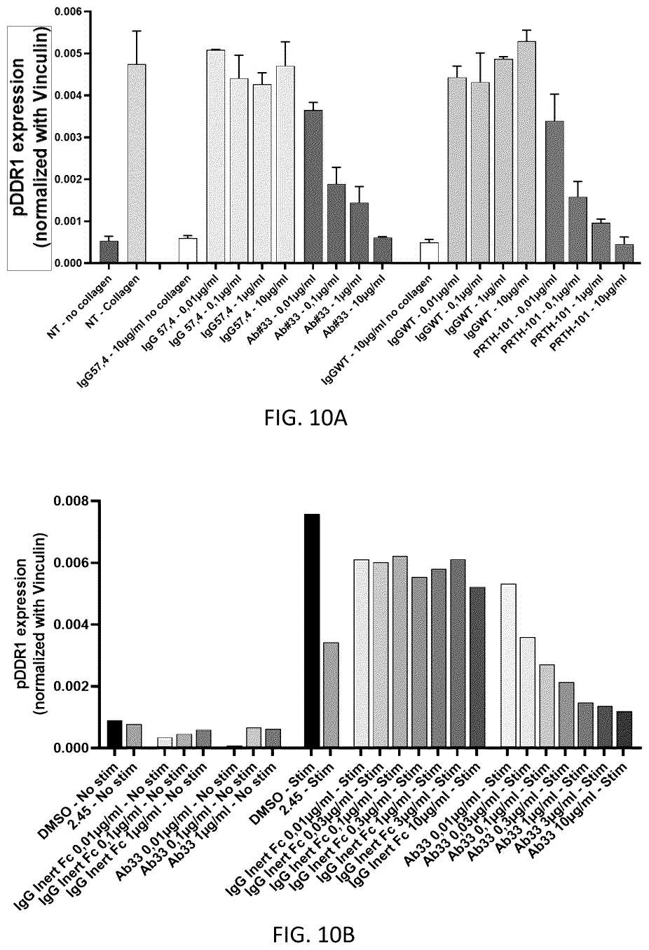

Serum-starved T47D cells were pretreated for two hours with increasing concentrations of monoclonal antibody or IgG control, followed by stimulation with 50 μg/ml of human collagen I for 90 minutes. As shown in FIG. 10A, rabbit mAb #33 was able to inhibit collagen I-induced phosphorylation of DDR1, in a similar pattern and with a comparable potency to 9H1-WT (PRTH-101). As shown in FIG. 10B, chimeric rabbit/human mAb #33, comprising rabbit mAb #33 heavy and light chain variable domains fused to human IgG1 heavy and light chain constant domains (See Table 4), produced results similar to 9H1-WT and rabbit mAb #33.

### 8.2 Example 2: In Vivo Characterization of Anti-DDR1 mAbs

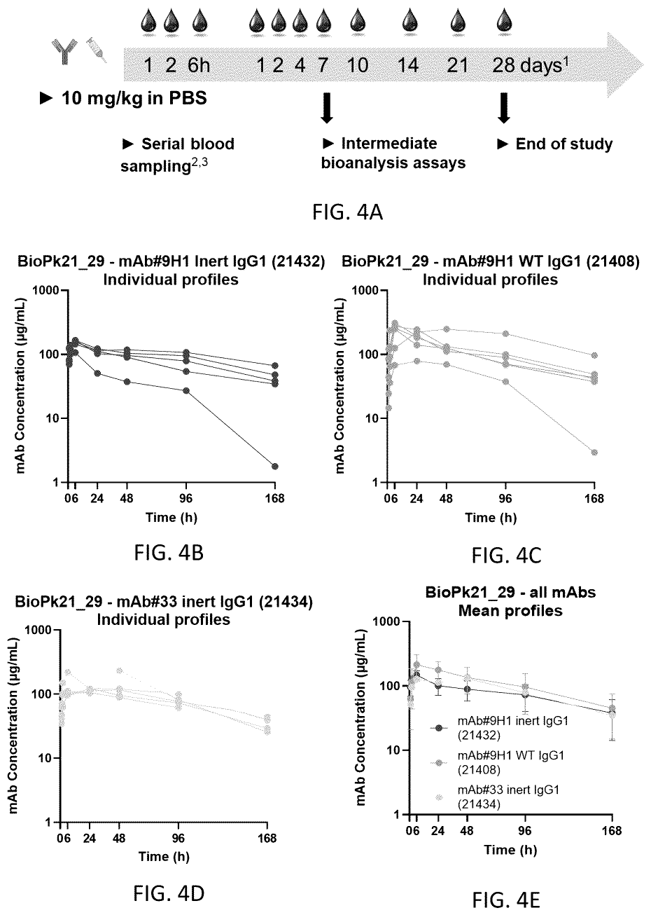

To evaluate the pharmacokinetic profiles of anti-DDR1 mAbs, mAbs were administered intraperitoneally to female C57B16JrJ mice at a dose of 10 mg/kg, serial blood sampling was performed over time (FIG. 4A), and ELISA was used to measure circulating free and partially bound antibody. As shown in FIGS. 4B-4D, exposures for humanized mAb #9H1 with Inert IgG1Fc (FIG. 4B), humanized mAb #9H1 with WT IgG1Fc (FIG. 4C) and chimeric rabbit/human mAb #33 with inert IgG1 Fc (FIG. 4D) were comparable to expected exposures for IgG1. Further, as shown in FIG. 4E and Table S1 below, the maximal circulating (free and partially bound) concentrations of each mAb were higher than the concentration required to bind to target (as shown by the surface plasmon resonance results of mAb binding to mouse DDR1 extracellular domain; see Table S2 below). Moreover, the maximal circulating (free and partially bound) concentrations of mAb #9H1 WT IgG1 were higher than the in vitro concentration required to inhibit Collagen I-induced phosphorylation of DDR1 in T47D cells (see FIG. 1).

TABLE S1
Pharmacokinetic parameters for anti-DDR1 mAbs.

| | |
|---|---|
| Cmax (μg/mL) | tₘₐₓ (h) |

| | | | |
|---|---|---|---|
| #9H1 inert IgG1 | 147.28 | (810 nM) | 6 |
| #9H1 WT IgG1 | 214.53 | (1200 nM) | 6 |
| #33 inert IgG1 | 131.82 | (650 nM) | 48 |

TABLE S2
SPR binding characterization of mAbs
for mouse DDR1 extracellular domain.

| mAb | kon (1/Ms) | Koff (1/s) | KD (nM) |
|---|---|---|---|
| mAb #33 inert IgG1 Fc | 1.05E+06 | 1.06E−03 | 1.01 |
| mAb #9H1 inert IgG1 Fc | 1.97E+05 | 3.75E−03 | 19.0 |
| mAb #9H1 WT IgG1 Fc | 2.14E+05 | 3.77E−03 | 17.6 |

### 8.3 Example 3: Characterizing DDR1 Phosphorylation in Skin Samples

An assay was developed to detect pDDR1 in skin samples. Initially, pDDR1 was detected in reference human skin samples from healthy subjects, as described below. Strikingly, the pDDR1 detected in these reference samples is almost exclusively a cleaved form of DDR1.

Healthy human subject 4 mm fresh frozen skin punch biopsy samples were obtained from Discovery Life Sciences. Sample 1 was a normal skin sample from a white female aged 36, collected via abdominoplasty on Jul. 13, 2022 (Patient ID: 122299014). Sample 2 was a normal skin sample from a white female aged 51, collected via abdominoplasty on Jul. 20, 2022 (Patient ID: 122305452).

To extract protein, the samples were removed from −80° C. freezer and suspended in 2 ml Pierce RIPA buffer (Thermo)+1× Halt™ Protease/Phosphatase Inhibitor Cocktail (Thermo)+5 mM EDTA+5 nM Batimastat (R&D Systems) in GentleMacs M Tube (Miltenyi). The samples were cycled twice on the Protein 01_01 cycle on a GentleMacs Disassociator (Miltenyi) at 4° C. and spun for 1 min 30 sec at 220 Rcf at 4° C. to dissipate foam and collect sample. 300 μL of whole skin lysate was removed (2 Cycle Skin Lysate sample). 200 μL of 10% SDS was added to 300 μL of whole skin lysate and the mixture was boiled at 95° C. for 5 min (2 Cycle Skin Lysate 4% SDS sample). The remaining lysate was centrifuged for 4 min at 220 Rcf at 4° C., and supernatant was removed (2 Cycle Skin Supernatant sample). The remaining lysate was run on the Protein01_01 cycle for a third time and centrifuged for 4 min at 220 Rcf at 4° C. Supernatant was removed (3 Cycle Skin Supernatant sample). The remaining lysate was resuspended, and 200 μL of 10% SDS was added to 300 μL of lysate, and the mixture was boiled as described above (3 Cycle Lysate 4% SDS sample). Finally, approximately 300 μL of the remaining lysate was recovered (3 Cycle Skin Lysate sample).

All lysates were boiled at 95° C. for 20 min to resolubilize protein, and 200 μg/mL samples were prepared with 0.1× Sample Buffer. Phosphorylated DDR1 was detected in the samples using the Jess Total Protein Detection Chemiluminescence Assay, incorporating the 12-230 kDa Fluorescence Separation Module, the Replex Module, the Anti-Rabbit Detection Module, and the Total Protein Detection Module (all from Protein Simple) and the Phospho-DDR1 (Tyr513) (E1N8F) antibody (Cell Signaling; mAb Cat. #5583).

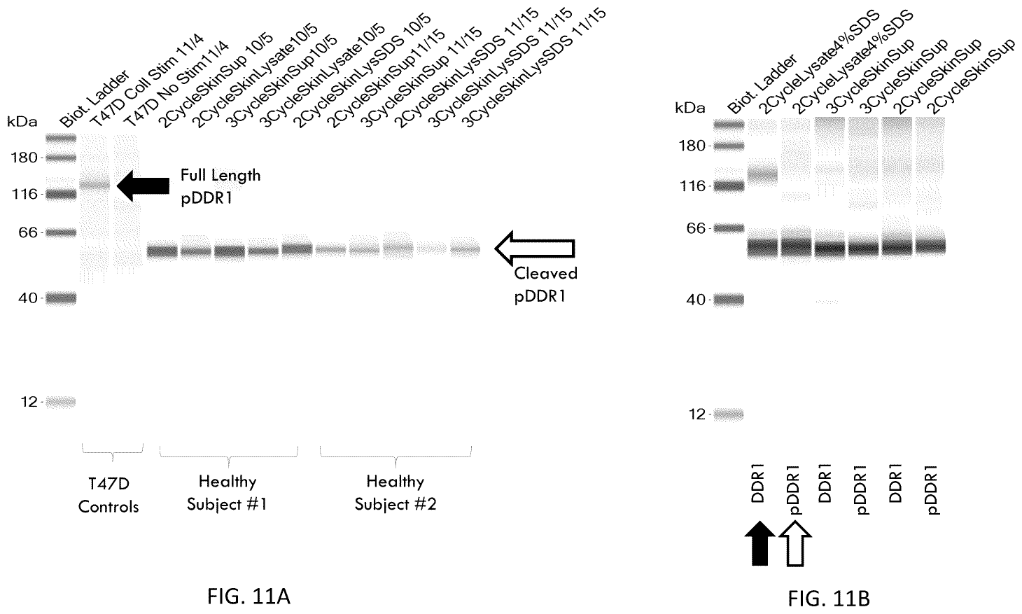

As shown in FIG. 11A, pDDR1 was detected in all preparations of skin lysates from both healthy subjects. Interestingly, only cleaved intracellular pDDR1 (approximately 60 kDa) was detected in the skin lysate samples (FIG. 11A, indicated by white arrow). In contrast, T47D breast cancer cells stimulated with collagen (e.g., as described above) show only full-length pDDR1 (approximately 125 kDa; FIG. 11A, indicated by black arrow).

The results described above do not indicate that full-length DDR1 is not present in the samples assayed, as the antibody used is specific to pDDR1. Use of the DDR1 (D1G6) antibody (Cell Signaling), which is not specific to pDDR1, indicates that both full-length and cleaved DDR1 can be detected in skin lysate samples (FIG. 11B, see lane indicated by black arrow). However, only the cleaved form is phosphorylated (FIG. 11B, see lane indicated by white arrow).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. A method of monitoring the effectiveness of an anti-discoidin domain receptor tyrosine kinase 1 (DDR1) antibody or antigen-binding fragment thereof in a subject in need thereof comprising
a) administering an effective amount of the anti-DDR1 antibody to the subject; and
b) detecting a level of DDR1 phosphorylation in a sample from the subject,
wherein a decrease in DDR1 phosphorylation in the sample from the subject in comparison to a positive reference sample indicates that the administration of the anti-DDR1 antibody is effective.

2. The method of claim 1, wherein the subject has cancer.

3. The method of claim 2, wherein the cancer is selected from the group consisting of: pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

4. The method of claim 1, wherein the subject has a fibrotic condition.

5. The method of claim 4, wherein the fibrotic condition is selected from the group consisting of: skin hypertrophic scarring, scleroderma, lung scarring, idiopathic pulmonary fibrosis, cirrhotic liver fibrosis, renal fibrosis, and interstitial lung disease.

6. A method of treating a DDR1 related disorder in a subject in need thereof comprising
a) administering an effective amount of an anti-DDR1 antibody or antigen-binding fragment thereof to the subject; and
b) detecting a level of DDR1 phosphorylation in a sample from the subject,
wherein a decrease in DDR1 phosphorylation in the sample from the subject in comparison to a positive reference sample indicates that the treatment is effective.

7. A method of screening for a subject with a DDR1 related disorder that is likely to be effectively treated with an anti-DDR1 antibody comprising detecting a level of DDR1 phosphorylation in a sample from the subject, wherein if DDR1 phosphorylation in the sample from the subject is higher in comparison to a negative reference sample, then the DDR1 related disorder is likely to be effectively treated with an anti-DDR1 antibody.

8. A method of treating a DDR1 related disorder in a subject in need thereof comprising
a) detecting a level of DDR1 phosphorylation in a sample from the subject; and
b) administering an effective amount of an anti-DDR1 antibody or antigen-binding fragment thereof to the subject if DDR1 phosphorylation in the sample from the subject is higher in comparison to a negative reference sample.

9. The method of any one of claims 6-8, wherein the DDR1 related disorder is cancer.

10. The method of claim 9, wherein the cancer is selected from the group consisting of: pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

11. The method of any one of claims 6-8, wherein the DDR1 related disorder is a fibrotic condition.

12. The method of claim 11, wherein the fibrotic condition is selected from the group consisting of: skin hypertrophic scarring, scleroderma, lung scarring, idiopathic pulmonary fibrosis, cirrhotic liver fibrosis, renal fibrosis, and interstitial lung disease.

13. The method of any one of claims 1-3, or 6-10, wherein the sample comprises tumor tissue.

14. The method of any one of claims 1-12, wherein the sample comprises one or more selected from the group consisting of: blood cells, skin tissue, lung tissue, renal tissue, and liver tissue.

15. The method of any one of claims 1-12, wherein the sample comprises a skin punch biopsy sample.

16. A method of screening for an anti-DDR1 antibody or antigen-binding fragment thereof that is effective in treating a DDR1 related disorder comprising
a) administering an effective amount of the anti-DDR1 antibody or antigen-binding fragment thereof to a cell; and
b) detecting a level of DDR1 phosphorylation in the cell,
wherein a decrease in DDR1 phosphorylation in the cell in comparison to a positive reference cell indicates that the anti-DDR1 antibody or antigen-binding fragment thereof is effective in treating the DDR1 related disorder.

17. A method of screening for an anti-DDR1 antibody or antigen-binding fragment thereof that is effective in reducing collagen interaction with a cell comprising
a) administering an effective amount of the anti-DDR1 antibody or antigen-binding fragment thereof to the cell; and
b) detecting a level of DDR1 phosphorylation in the cell,
wherein a decrease in DDR1 phosphorylation in the cell in comparison to a positive reference cell indicates that the anti-DDR1 antibody or antigen-binding fragment thereof is effective in reducing collagen interaction with the cell.

18. The method of claim 16 or 17, wherein the cell is a cancer cell.

19. The method of claim 18, wherein the cancer cell is derived from a cancer selected from the group consisting of: pancreatic cancer; lung cancer, including small cell lung cancer and non-small cell lung cancer; colon and colorectal cancer; head and neck cancer; stomach (gastric) cancer; ovarian cancer; breast cancer; kidney cancer; liver cancer; prostate cancer; cervical cancer; brain cancer; skin cancer, including melanoma; sarcoma; cholangiocarcinoma; and bone cancer.

20. The method of claim 16 or 17, wherein the cell is one or more selected from the group consisting of: a skin cell, a lung cell, a kidney cell, and a liver cell.

21. The method of any one of claims 1-20, wherein the anti-DDR1 antibody or antigen-binding fragment thereof comprises a heavy chain variable domain (VH) comprising the CDRH1, CDRH2 and CDRH3 amino acid sequences of the VH amino acid sequence of SEQ ID NO: 4 or 13 and a light chain variable domain (VL) comprising the CDRL1, CDRL2 and CDRL3 amino acid sequences of the VL amino acid sequence of SEQ ID NO: 3, 11, or 12.

22. The method of claim 21, wherein
a) the CDRL1 comprises the amino acid sequence of SEQ ID NO: 5;
b) the CDRL2 comprises the amino acid sequence of QAS;
c) the CDRL3 comprises the amino acid sequence of SEQ ID NO: 7;
d) the CDRH1 comprises the amino acid sequence of SEQ ID NO: 8;
e) the CDRH2 comprises the amino acid sequence of SEQ ID NO: 9; and
f) the CDRH3 comprises the amino acid sequence of SEQ ID NO: 10.

23. The method of claim 21, wherein
a) the CDRL1 comprises the amino acid sequence of SEQ ID NO: 17;
b) the CDRL2 comprises the amino acid sequence of GVF;
c) the CDRL3 comprises the amino acid sequence of SEQ ID NO: 19;
d) the CDRH1 comprises the amino acid sequence of SEQ ID NO: 20;
e) the CDRH2 comprises the amino acid sequence of SEQ ID NO: 21; and
f) the CDRH3 comprises the amino acid sequence of SEQ ID NO: 22.

24. The method of claim 22 or 23, wherein the anti-DDR1 antibody comprises:
a) a VL domain comprising an amino acid sequence that is at least 90% identical to an amino acid sequence selected from the group consisting of SEQ ID NOs: 3, 11, and 12; and
b) a VH domain comprising an amino acid sequence that is at least 90% identical to an amino acid sequence selected from the group consisting of SEQ ID NOs: 4 and 13.

25. The method of claim 22 or 23, wherein the anti-DDR1 antibody comprises:
a) a VL domain comprising an amino acid sequence selected from the group consisting of SEQ ID NOs: 3, 11, and 12; and
b) a VH domain comprising an amino acid sequence selected from the group consisting of SEQ ID NOs: 4 and 13.

26. The method of claim 25, wherein the anti-DDR1 antibody comprises a VL domain and a VH domain selected from the group consisting of:
a) SEQ ID NOs: 3 and 4, respectively;
b) SEQ ID NOs: 11 and 13, respectively; and
c) SEQ ID NOs: 12 and 13, respectively.

27. The method of claim 26, wherein the anti-DDR1 antibody comprises a VL domain and a VH domain comprising the amino acid sequence of SEQ ID NOs: 3 and 4, respectively.

28. The method of claim 26, wherein the anti-DDR1 antibody comprises a VL domain and a VH domain comprising the amino acid sequence of SEQ ID NOs: 11 and 13, respectively.

29. The method of claim 26, wherein the anti-DDR1 antibody comprises a VL domain and a VH domain comprising the amino acid sequence of SEQ ID NOs: 12 and 13, respectively.

30. The method of any one of claims 1-29, wherein detecting the level of DDR1 phosphorylation comprises detecting the level of phosphorylation of a cleaved form of DDR1.

31. The method of claim 30, wherein the cleaved form of DDR1 has a molecular weight of approximately 65 kDa.
